SEQUENCE LISTING (124 sequences)

SEQ ID NO: 1 - protein, 98 aa, Homo Sapiens
QSVLTQPPSVSEAPRQRVTISCSGSSSNIGNNAVNWYQQLPGKAPKLLIYYDDLLPSGVSDRFSGSKSGTSASLAISGLQSEDEADYYCAAWDDSLNG

SEQ ID NO: 2 - protein, 99 aa, Homo Sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSG

SEQ ID NO: 3 - protein, 98 aa, Homo Sapiens
QSVLTQPPSVSAAPGQKVTISCSGSSSDMGNYAVSWYQQLPGTAPKLLIYENNKRPSGIPDRFSGSKSGTSATLGITGLWPEDEADYYCLAWDTSPRA

SEQ ID NO: 4 - protein, 98 aa, Homo Sapiens
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNG

SEQ ID NO: 5 - protein, 98 aa, Homo Sapiens
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSG

SEQ ID NO: 6 - protein, 99 aa, Homo Sapiens
QSVLTQPPSVSGAPGQRVTISCTGSSSNIGAGYVVHWYQQLPGTAPKLLIYGNSNRPSGVPDQFSGSKSGTSASLAITGLQSEDEADYYCKAWDNSLNA

SEQ ID NO: 7 - protein, 98 aa, Homo Sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSA

SEQ ID NO: 8 - protein, 97 aa, Homo Sapiens
QSVLTQPPSVSWATRQRLTVSCTGSSSNTGTGYNVNCWQLPRTDPKLLRHGDKNWASWVSDQFSGSKSGSLASLGTTGLWAEDKTDYHCQSRDICVL

SEQ ID NO: 9 - protein, 97 aa, Homo Sapiens
QSALIQPPSVSGSPGQSVTISCTGTSSDVGSYDYVSWYQQHPGTVPKPMIYNVNTQPSGVPDRFSGSKSGNTASMTISGLQAEDEADYCCSYTSSAT

SEQ ID NO: 10 - protein, 99 aa, Homo Sapiens
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNF

SEQ ID NO: 11 - protein, 99 aa, Homo Sapiens
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTF

SEQ ID NO: 12 - protein, 99 aa, Homo Sapiens
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTL

SEQ ID NO: 13 - protein, 99 aa, Homo Sapiens
QSALTQPPSVSGSPGQSVTISCTGTSSDVGSYNRVSWYQQPPGTAPKLMIYEVSNRPSGVPDRFSGSKSGNTASLTISGLQAEDEADYYCSLYTSSSTF

SEQ ID NO: 14 - protein, 99 aa, Homo Sapiens
QSALTQPASVSGSPGQSITISCTGTSSDVGSYNLVSWYQQHPGKAPKLMIYEGSKRPSGVSNRFSGSKSGNTASLTISGLQAEDEADYYCCSYAGSSTL

SEQ ID NO: 15 - protein, 99 aa, Homo Sapiens
QSALTQPPFVSGAPGQSVTISCTGTSSDVGDYDHVFWYQKRLSTTSRLLIYNVNTRPSGISDLFSGSKSGNMASLTISGLKSEVEANYHCSLYSSSYTF

SEQ ID NO: 16 - protein, 97 aa, Homo Sapiens
QSVLTQPRSVSRSPGQVTIFCTGTSSDIGGYDLVSWCQHPGKAPKLMIYDVANWPSGAPGCFSGSKSGNTASLTISGLQAEDEADYYCSSYAGSYNF

SEQ ID NO: 17 - protein, 97 aa, Homo Sapiens
QSVLTQPRSVSRSPGQVTIFCTGTSSDIGGYDLVSWCQHPGKAPKLMIYDVGNWPSGAPGCFSGSKSGNTASLTISGLQAEDEADYYCSSYAGSYNF

SEQ ID NO: 18 - protein, 95 aa, Homo Sapiens
SYELTQPPSVSVSPGQTASITCSGDKLGDKYACWYQQKPGQSPVLVIYQDSKRPSGIPERFSGSNSGNTATLTISGTQAMDEADYYCQAWDSSTA

SEQ ID NO: 19 - protein, 95 aa, Homo Sapiens
SYELTQPLSVSVALGQTARITCGGNNIGSKNVHWYQQKPGQAPVLVIYRDSNRPSGIPERFSGSNSGNTATLTISRAQAGDEADYYCQVWDSSTA

SEQ ID NO: 20 - protein, 96 aa, Homo Sapiens
SYELTQPPSVSVSPGQTARITCSGDALPKKYAYWYQQKSGQAPVLVIYEDSKRPSGIPERFSGSSSGTMATLTISGAQVEDEADYYCYSTDSSGNH

SEQ ID NO: 21 - protein, 96 aa, Homo Sapiens
SYELTQPHSVSVATAQMARITCGGNNIGSKAVHWYQQKPGQDPVLVIYSDSNRPSGIPERFSGSNPGNTTTLTISRIEAGDEADYYCQVWDSSSDH

SEQ ID NO: 22 - protein, 93 aa, Homo Sapiens
SYELTQPPAVSVSPGQTARISCSGDVLRDNYADWYPQKPGQAPVLVIYKDGERPSGIPERFSGSTSGNTTALTISRVLTKGGADYYCFSGDNN

SEQ ID NO: 23 - protein, 96 aa, Homo Sapiens
SYELTQPPSVSVSLGQMARITCSGEALPKKYAYWYQQKPGQFPVLVIYKDSERPSGIPERFSGSSSGTIVTLTISGVQAEDEADYYCLSADSSGTY

SEQ ID NO: 24 - protein, 96 aa, Homo Sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNH

SEQ ID NO: 25 - protein, 96 aa, Homo Sapiens
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDH

SEQ ID NO: 26 - protein, 94 aa, Homo Sapiens
SYELTQLPSVSVSPGQTARITCSGDVLGENYADWYQQKPGQAPELVIYEDSERYPGIPERFSGSTSGNTTTLTISRVLTEDEADYYCLSGDEDN

SEQ ID NO: 27 - protein, 96 aa, Homo Sapiens
SYELMQPPSVSVSPGQTARITCSGDALPKQYAYWYQQKPGQAPVLVIYKDSERPSGIPERFSGSSSGTTVTLTISGVQAEDEADYYCQSADSSGTY

SEQ ID NO: 28 - protein, 94 aa, Homo Sapiens
SYELTQPSSVSVSPGQTARITCSGDVLAKKYARWFQQKPGQAPVLVIYKDSERPSGIPERFSGSSSGTTVTLTISGAQVEDEADYYCYSAADNN

SEQ ID NO: 29 - protein, 94 aa, Homo Sapiens
SSELSQEPAVSVALGTARITCQGDSIEDSVVNWYKQKPSQAPGLVILNSVQSSGIPKKFSGSSSGNMATLTITGIQVEDKADYYCQSWDSSRTH

SEQ ID NO: 30 - protein, 94 aa, Homo Sapiens
SSGPTQVPAVSVALGQMARITCQGDSMEGSYEHWYQQKPGQAPVLVIYDSSDRPSRIPERFSGSKSGNTTTLTITGAQAEDEADYYYQLIDNHA

SEQ ID NO: 31 - protein, 103 aa, Homo Sapiens
LPVLTQPPSASALLGASIKLTCTLSSEHSTYTIEWYQQRPGRSPQYIMKVKSDGSHSKGDGIPDRFMGSSSGADRYLTFSNLQSDDEAEYHCGESHTIDGQVG

SEQ ID NO: 32 - protein, 99 aa, Homo Sapiens
QPVLTQSSSASASLGSSVKLTCTLSSGHSSYIIAWHQQQPGKAPRYLMKLEGSGSYNKGSGVPDRFSGSSSGADRYLTISNLQLEDEADYYCETWDSNT

SEQ ID NO: 33 - protein, 99 aa, Homo Sapiens
QLVLTQSPSASASLGASVKLTCTLSSGHSSYAIAWHQQQPEKGPRYLMKLNSDGSHSKGDGIPDRFSGSSSGAERYLTISSLQSEDEADYYCQTWGTGI

SEQ ID NO: 34 - protein, 104 aa, Homo Sapiens
QPVLTQPPSSSASPGESARLTCTLPSDINVGSYNIYWYQQKPGSPPRYLLYYYSDSDKGQGSGVPSRFSGSKDASANTGILLISGLQSEDEADYYCMIWPSNAS

SEQ ID NO: 35 - protein, 104 aa, Homo Sapiens
QPVLTQPTSLSASPGASARFTCTLRSGINVGTYRIYWYQQKPGSLPRYLLRYKSDSDKQQGSGVPSRFSGSKDASTNAGLLLISGLQSEDEADYYCAIWYSSTS

SEQ ID NO: 36 - protein, 104 aa, Homo Sapiens
QAVLTQPASLSASPGASASLTCTLRSGINVGTYRIYWYQQKPGSPPQYLLRYKSDSDKQQGSGVPSRFSGSKDASANAGILLISGLQSEDEADYYCMIWHSSAS

SEQ ID NO: 37 - protein, 104 aa, Homo Sapiens
QPVLTQPTSLSASPGASARLTCTLRSGINLGSYRIFWYQQKPESPPRYLLSYYSDSSKHQGSGVPSRFSGSKDASSNAGILVISGLQSEDEADYYCMIWHSSAS

SEQ ID NO: 38 - protein, 105 aa, Homo Sapiens
QPVLTQPSSHSASSGASVRLTCMLSSGFSVGDFWIRWYQQKPGNPPRYLLYYHSDSNKGQGSGVPSRFSGSNDASANAGILRISGLQPEDEADYYCGTWHSNSKT

SEQ ID NO: 39 - protein, 98 aa, Homo Sapiens
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSN

SEQ ID NO: 40 - protein, 98 aa, Homo Sapiens
QTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQ

SEQ ID NO: 41 - protein, 98 aa, Homo Sapiens
QAVVTQEPSLTVSPGGTVTLTCGSSTGAVTSGHYPYWFQQKPGQAPRTLIYDTSNKHSWTPARFSGSLLGGKAALTLSGAQPEDEAEYYCLLSYSGAR

SEQ ID NO: 42 - protein, 98 aa, Homo Sapiens
QTVVTQEPSFSVSPGGTVTLTCGLSSGSVSTSYYPSWYQQTPGQAPRTLIYSTNTRSSGVPDRFSGSILGNKAALTITGAQADDESDYYCVLYMGSGI

SEQ ID NO: 43 - protein, 104 aa, Homo Sapiens
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFV

SEQ ID NO: 44 - protein, 98 aa, Homo Sapiens
QAGLTQPPSVSKGLRQTATLTCTGNSNNVGNQGAAWLQQHQGHPPKLLSYRNNNRPSGISERLSASRSGNTASLTITGLQPEDEADYYCSAWDSSLSA

SEQ ID NO: 45 - protein, 104 aa, Homo Sapiens
RPVLTQPPSLSASPGATARLPCTLSSDLSVGGKNMFWYQQKPGSSPRLFLYHYSDSDKQLGPGVPSRVSGSKETSSNTAFLLISGLQPEDEADYYCQVYESSAN

SEQ ID NO: 46 - protein, 14 aa, Homo Sapiens
QSVLTQPPSVSEAP

SEQ ID NO: 47 - protein, 11 aa, Homo Sapiens
RQRVTISCSGS

SEQ ID NO: 48 - protein, 8 aa, Homo Sapiens
SSNIGNNA

SEQ ID NO: 49 - protein, 8 aa, Homo Sapiens
VNWYQQLP

SEQ ID NO: 50 - protein, 9 aa, Homo Sapiens
GKAPKLLIY

SEQ ID NO: 51 - protein, 8 aa, Homo Sapiens
LLPSGVSD

SEQ ID NO: 52 - protein, 8 aa, Homo Sapiens
RFSGSKSG

SEQ ID NO: 53 - protein, 12 aa, Homo Sapiens
TSASLAISGLQS

SEQ ID NO: 54 - protein, 8 aa, Homo Sapiens
EDEADYYC

SEQ ID NO: 55 - protein, 9 aa, Homo Sapiens
AAWDDSLNG

SEQ ID NO: 56 - DNA, 290 bp, Homo Sapiens
tcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggatagtagtagtgatcatcc

SEQ ID NO: 57 - DNA, 290 bp, Homo Sapiens
tcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggacagtagtagtgatcatcc

SEQ ID NO: 58 - DNA, 290 bp, Homo Sapiens
tcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatcc

SEQ ID NO: 59 - DNA, 290 bp, Homo Sapiens
tcctatgtgctgactcagccaccctcggtgtcagtggccccaggaaagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatcc

SEQ ID NO: 60 - protein, 19 aa, Homo Sapiens
MAWTALLLGLLSHCTGSVT

SEQ ID NO: 61 - DNA, 57 bp, Homo Sapiens
atggcctggaccgctctcctcctcggcctcctctctcactgcacaggctctgtgacc

SEQ ID NO: 62 - protein, 20 aa, Mus Musculus
MSVPTQVLGLLLLWLTDARC

SEQ ID NO: 63 - DNA, 60 bp, Mus Musculus
atgagtgtgcccactcaggtcctggggttgctgctgctgtggcttacagatgccagatgt

SEQ ID NO: 64 - DNA, 60 bp, Artificial Sequence
  nucleotide sequence encoding mouse kappa light chain signal peptide, codon-optimised for CHO
atgtctgtgcctacacaggttctgggactgctgctgctgtggctgaccgacgccagatgt

SEQ ID NO: 65 - DNA, 381 bp, Artificial Sequence
  nucleic acid encoding 0325L VL domain with signal peptide
atgtctgtgcctacacaggttctgggactgctgctgctgtggctgaccgacgccagatgttacgtgctgacccagcctccttccgtgtctgttgctcctggcgagacagccagaatcacctgtggcggcgataacatcggccggaagtccgtgtactggtatcagcagaagtccggccaggctcctgtgctggtcatctactacgactccgaccggccttctggcatccctgagagattctccggctccaactccggcaataccgccacactgaccatctccagagtggaagctggcgacgaggccgactactactgccaagtgtgggacggctcctctgaccactgggttttcggcggaggcaccaagctgacagtgctg

SEQ ID NO: 66 - protein, 127 aa, Artificial Sequence
  0325L with mouse kappa signal peptide
MSVPTQVLGLLLLWLTDARCYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVL

SEQ ID NO: 67 - protein, 233 aa, Artificial Sequence
  Lambda light chain comprising 0325L VL domain with mouse kappa signal peptide
MSVPTQVLGLLLLWLTDARCYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 68 - protein, 128 aa, Artificial Sequence
  0128L VL domain with mouse kappa signal peptide
MSVPTQVLGLLLLWLTDARCSYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVL

SEQ ID NO: 69 - protein, 18 aa, Artificial Sequence
  V12 signal peptide derived from Sandfly Yellow related protein
MRFFFVFLAIVLFQGIHG

SEQ ID NO: 70 - protein, 15 aa, Artificial Sequence
  V14 signal peptide derived from Silkworm Fibroin LC
MKPIFLVLLWTSAYA

SEQ ID NO: 71 - protein, 16 aa, Artificial Sequence
  V16 signal peptide derived from Snake PLA2
MRTLWIMAVLLLGVEG

SEQ ID NO: 72 - protein, 18 aa, Artificial Sequence
  V17 signal peptide derived from Cypridina Noctiluca luciferase
MKTLILAVALVYCATVHC

SEQ ID NO: 73 - protein, 17 aa, Artificial Sequence
  V19 signal peptide derived from Pinemoth Fibroin LC
MMRPIVLVLLFATSALA

SEQ ID NO: 74 - protein, 19 aa, Mus Musculus
MGWSCIILFLVATATGVHS

SEQ ID NO: 75 - protein, 15 aa, Homo Sapiens
AWTPLLLPLLTLCTS

SEQ ID NO: 76 - protein, 19 aa, Homo Sapiens
MAGFPLLLTLLTHCAGSWA

SEQ ID NO: 77 - protein, 22 aa, Homo Sapiens
MDMRVPAQLLGLLLLWLPGAKC

SEQ ID NO: 78 - DNA, 366 bp, Homo Sapiens
gaggtgcagctggtggagtctgggggaggctttgtccagcctggggggtccctgagactctcctgtgcagtctctggattcagatttaatagctattggatgagctgggtccgccaggctccagggaaggggctggagtgggtggccaacataaaccaagatggaagtagaaaattctatgtggcctctgtgaagggccgattcaccatgtccagagacaacgccaagaaatcagtgtatgtacaaatgaacagcctgagagccgaggacacggctgtgtattactgtgcgagagaggggtatagtagtatcaagtattatggtatggacgtctggggccaagggaccacggtcaccgtctcctca

SEQ ID NO: 79 - protein, 122 aa, Homo Sapiens
EVQLVESGGGFVQPGGSLRLSCAVSGFRFNSYWMSWVRQAPGKGLEWVANINQDGSRKFYVASVKGRFTMSRDNAKKSVYVQMNSLRAEDTAVYYCAREGYSSIKYYGMDVWGQGTTVTVSS

SEQ ID NO: 80 - DNA, 1341 bp, Artificial Sequence
  Nucleic acid encoding N1280H-IgG4-P K439E heavy chain; N1280 coding sequence underlined
gaagtgcagctggttgaatctggcggcggatttgttcagcctggcggctctctgagactgtcctgtgctgtgtccggcttccggttcaactcctactggatgtcctgggtccgacaggctcctggcaaaggactggaatgggtcgccaacatcaaccaggacggctcccggaagttctacgtggcctctgtgaagggcagattcaccatgtctcgggacaacgccaagaaatccgtgtacgtgcagatgaactccctgagagccgaggacaccgccgtgtactactgtgctagagagggctactcctccatcaagtactacggcatggacgtgtggggccagggcacaaccgtgacagtctcttccgcttccaccaagggacccagcgttttccctctggctccttgctccagatccacctccgagtctacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacacctgtaatgtggaccacaagccttccaacaccaaggtggacaagcgcgtggaatctaagtacggccctccttgtcctccatgtcctgctccagagtttctcggcggaccctccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaatgccaagaccaagcctagagaggaacagtacaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagcctcgagaaccccaggtttacaccctgcctccaagccaagaggaaatgaccaagaaccaggtgtccctgacctgtctcgtgaagggcttctacccctccgatatcgccgtggaatgggagtctaatggccagccagagaacaactacaagaccacacctccagtgctggactccgacggctcattctttctgtactccaagctgacagtggacaagtcccggtggcaagagggcaacgtgttctcctgctctgtgatgcacgaggccctgcacaaccactacacccaagagtccctgtctctgtcccct

SEQ ID NO: 81 - protein, 447 aa, Artificial Sequence
  N1280H-IgG4-P K439E amino acid sequence
EVQLVESGGGFVQPGGSLRLSCAVSGFRFNSYWMSWVRQAPGKGLEWVANINQDGSRKFYVASVKGRFTMSRDNAKKSVYVQMNSLRAEDTAVYYCAREGYSSIKYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 82 - DNA, 366 bp, Homo Sapiens
gaggtgcagctggttgaatctggcggcggatttgttcagcctggcggctctctgagactgagctgtgccgtgtccggcttccggttcaacagctactggatgtcctgggtccgacaggcccctggcaaaggacttgagtgggtcgccaacatcaaccaggacggcagccggaagttttacgtggcctctgtgaagggcagattcaccatgagccgggacaacgccgacaaaagcgtgtacgtgcagatgaacagcctgagagccgaggacaccgccgtgtactattgtgccagagagggctacagcagcatcaagtactacggcatggacgtgtggggccagggcacaacagtgacagtctcttct

SEQ ID NO: 83 - protein, 122 aa, Homo Sapiens
EVQLVESGGGFVQPGGSLRLSCAVSGFRFNSYWMSWVRQAPGKGLEWVANINQDGSRKFYVASVKGRFTMSRDNADKSVYVQMNSLRAEDTAVYYCAREGYSSIKYYGMDVWGQGTTVTVSS

SEQ ID NO: 84 - DNA, 1341 bp, Artificial Sequence
  Nucleic acid encoding N1441H-IgG4-P K439E heavy chain
gaagtgcagctggttgaatctggcggcggatttgttcagcctggcggctctctgagactgtcctgtgctgtgtccggcttccggttcaactcctactggatgtcctgggtccgacaggctcctggcaaaggactggaatgggtcgccaacatcaaccaggacggctcccggaagttctacgtggcctctgtgaagggcagattcaccatgtctcgggacaacgccgacaagtccgtgtacgtgcagatgaactccctgagagccgaggacaccgccgtgtactactgtgctagagagggctactcctccatcaagtactacggcatggacgtgtggggccagggcacaaccgtgacagtctcttccgcttccaccaagggacccagcgttttccctctggctccttgctccagatccacctccgagtctacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacacctgtaatgtggaccacaagccttccaacaccaaggtggacaagcgcgtggaatctaagtacggccctccttgtcctccatgtcctgctccagagtttctcggcggaccctccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagcctcgagaaccccaggtttacaccctgcctccaagccaagaggaaatgaccaagaaccaggtgtccctgacctgtctcgtgaagggcttctacccctccgatatcgccgtggaatgggagtctaatggccagccagagaacaactacaagaccacacctccagtgctggactccgacggctcattctttctgtactccaagctgacagtggacaagtcccggtggcaagagggcaacgtgttctcctgctctgtgatgcacgaggccctgcacaaccactacacccaagagtccctgtctctgtcccct

SEQ ID NO: 85 - protein, 447 aa, Artificial Sequence
  N1441H-IgG4-P K439E amino acid sequence
EVQLVESGGGFVQPGGSLRLSCAVSGFRFNSYWMSWVRQAPGKGLEWVANINQDGSRKFYVASVKGRFTMSRDNADKSVYVQMNSLRAEDTAVYYCAREGYSSIKYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 86 - DNA, 363 bp, Homo Sapiens
caggttcagctgattcagtccggcgccaaagtgaagaaacctggcgcctctgtgaaggtgtcctgcaaggcctctcggtacaagttcacctcctactacatgcactgggtccgacaggcccctggacaaggattggagtggatgggcatcatcaaccccaagtccggctccacctcttacgcccagaaattccagggcagagtgaccatgaccagagacacctctacctccaccgtgtacatggaactgtccagcctgagatccgaggacaccgccgtgtactactgtgccagagatggctacggcagcttctccagactgatccagttgtggggccagggcacactggtcacagtgtcctct

SEQ ID NO: 87 - protein, 121 aa, Homo Sapiens
QVQLIQSGAKVKKPGASVKVSCKASRYKFTSYYMHWVRQAPGQGLEWMGIINPKSGSTSYAQKFQGRVTMTRDTSTSTVYMELSSLRSEDTAVYYCARDGYGSFSRLIQLWGQGTLVTVSS

SEQ ID NO: 88 - DNA, 1338 bp, Artificial Sequence
  Nucleic acid encoding T0999H-IgG4-P E356K heavy chain; T0999H coding sequence underlined
caggttcagctgattcagtccggcgccaaagtgaagaaacctggcgcctctgtgaaggtgtcctgcaaggcctctcggtacaagttcacctcctactacatgcactgggtccgacaggcccctggacaaggattggagtggatgggcatcatcaaccccaagtccggctccacctcttacgcccagaaattccagggcagagtgaccatgaccagagacacctctacctccaccgtgtacatggaactgtccagcctgagatccgaggacaccgccgtgtactactgtgccagagatggctacggcagcttctccagactgatccagttgtggggccagggcacactggtcacagtgtcctctgcttccaccaagggacccagcgtgttccctctggctccttgctccagatccacctccgagtctacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggaacccagacctacacctgtaatgtggaccacaagccttccaacaccaaggtggacaagcgcgtggaatctaagtacggccctccttgtcctccatgtcctgctccagagtttctcggcggaccctccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagcctcgagaaccccaggtttacaccctgcctccaagccagaaagagatgaccaagaaccaggtgtccctgacctgcctcgtgaagggcttctacccttccgatatcgccgtggaatgggagagcaatggccagccagagaacaactacaagaccacacctcctgtgctggactccgatggctcattctttctgtactccaagctgacagtggacaagtcccggtggcaagagggcaacgtgttctcctgctctgtgatgcacgaggccctgcacaaccactacacccagaagtccctgtctctgtcccct

SEQ ID NO: 89 - protein, 446 aa, Artificial Sequence
  T0999H-IgG4-P E356K amino acid sequence
QVQLIQSGAKVKKPGASVKVSCKASRYKFTSYYMHWVRQAPGQGLEWMGIINPKSGSTSYAQKFQGRVTMTRDTSTSTVYMELSSLRSEDTAVYYCARDGYGSFSRLIQLWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 90 - DNA, 363 bp, Homo Sapiens
caggtgcagttgatacagtctggggctgaggtgaagaagcctggggcctcagtgaaggtttcctgcaaggcatctagatacagcttcaccagctactatatgcactgggtgcgacaggcccctggacaagggcttgagtggatgggaataatcaaccctaaaagtggtagtacaagttacgcacagaagttccagggcagagtcaccatgaccagggacacgtccacgagcacagtctacatggagctgagcagcctgagatctgaggacacggccgtgtattactgtgcgagagatgggtatggcagctcgtcccggtgcctccagctctggggccagggcaccctggtcaccgtctcctca

SEQ ID NO: 91 - protein, 121 aa, Homo Sapiens
QVQLIQSGAEVKKPGASVKVSCKASRYSFTSYYMHWVRQAPGQGLEWMGIINPKSGSTSYAQKFQGRVTMTRDTSTSTVYMELSSLRSEDTAVYYCARDGYGSSSRCLQLWGQGTLVTVSS

SEQ ID NO: 92 - DNA, 363 bp, Homo Sapiens
caggtgcagttgatacagtctggggctgaggtgaagaagcctggggcctcagtgaaggtttcctgcaaggcatctagatacaagttcaccagctactatatgcactgggtgcgacaggcccctggacaagggcttgagtggatgggaataatcaaccctaaaagtggtagtacaagttacgcacagaagttccagggcagagtcaccatgaccagggacacgtccacgagcacagtctacatggagctgagcagcctgagatctgaggacacggccgtgtattactgtgcgagagatgggtatggcagcttctcccggctgatccagctctggggccagggcaccctggtcaccgtctcctca

SEQ ID NO: 93 - protein, 121 aa, Homo Sapiens
QVQLIQSGAEVKKPGASVKVSCKASRYKFTSYYMHWVRQAPGQGLEWMGIINPKSGSTSYAQKFQGRVTMTRDTSTSTVYMELSSLRSEDTAVYYCARDGYGSFSRLIQLWGQGTLVTVSS

SEQ ID NO: 94 - DNA, 1338 bp, Artificial Sequence
  Nucleic acid encoding T0736H-IgG4-P E356K heavy chain
caggttcagctgattcagtctggcgccgaagtgaagaaacctggcgcctctgtgaaggtgtcctgcaaggcctctcggtacaagttcacctcctactacatgcactgggtccgacaggcccctggacaaggattggagtggatgggcatcatcaaccccaagtccggctccacctcttacgcccagaaattccagggcagagtgaccatgaccagagacacctctacctccaccgtgtacatggaactgtccagcctgagatccgaggacaccgccgtgtactactgtgccagagatggctacggcagcttctccaggctgatccagttgtggggacagggcacactggtcaccgtgtcctctgcttctaccaagggacccagcgtgttccctctggctccttgctccagatccacctccgagtctacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggaacccagacctacacctgtaatgtggaccacaagccttccaacaccaaggtggacaagcgcgtggaatctaagtacggccctccttgtcctccatgtcctgctccagagtttctcggcggaccctccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagcctcgagaaccccaggtttacaccctgcctccaagccagaaagagatgaccaagaaccaggtgtccctgacctgcctcgtgaagggcttctacccttccgatatcgccgtggaatgggagagcaatggccagccagagaacaactacaagaccacacctcctgtgctggactccgatggctcattctttctgtactccaagctgacagtggacaagtcccggtggcaagagggcaacgtgttctcctgctctgtgatgcacgaggccctgcacaaccactacacccagaagtccctgtctctgtcccct

SEQ ID NO: 95 - protein, 446 aa, Artificial Sequence
  T0736H-IgG4-P E356K amino acid sequence
QVQLIQSGAEVKKPGASVKVSCKASRYKFTSYYMHWVRQAPGQGLEWMGIINPKSGSTSYAQKFQGRVTMTRDTSTSTVYMELSSLRSEDTAVYYCARDGYGSFSRLIQLWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 96 - DNA, 325 bp, Homo Sapiens
tcctatgtgctgactcagccaccctcagtgtcagtggccccaggagagacggccaggattacctgtgggggagacaacattggaaggaaaagtgtgtactggtaccagcagaagtcaggccaggcccctgtgctggtcatctattatgatagcgaccggccctcagggatccctgagcgattctctgggtccaactctgggaacacggcgaccctgaccatcagcagggtcgaagccggggatgaggccgactattactgtcaggtgtgggatggaagtagtgatcattgggtgttcggcggagggaccaagttgaccgtcctag

SEQ ID NO: 97 - protein, 108 aa, Homo Sapiens
SYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVL

SEQ ID NO: 98 - DNA, 642 bp, Homo Sapiens
tcctatgtgctgactcagccaccctcagtgtcagtggccccaggagagacggccaggattacctgtgggggagacaacattggaaggaaaagtgtgtactggtaccagcagaagtcaggccaggcccctgtgctggtcatctattatgatagcgaccggccctcagggatccctgagcgattctctgggtccaactctgggaacacggcgaccctgaccatcagcagggtcgaagccggggatgaggccgactattactgtcaggtgtgggatggaagtagtgatcattgggtgttcggcggagggaccaagttgaccgtcctaggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 99 - protein, 214 aa, Homo Sapiens
SYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 100 - DNA, 321 bp, Homo Sapiens
tacgtgctgacccagcctccttccgtgtctgttgctcctggcgagacagccagaatcacctgtggcggcgataacatcggccggaagtccgtgtactggtatcagcagaagtccggccaggctcctgtgctggtcatctactacgactccgaccggccttctggcatccctgagagattctccggctccaactccggcaataccgccacactgaccatctccagagtggaagctggcgacgaggccgactactactgccaagtgtgggacggctcctctgaccactgggttttcggcggaggcaccaagctgacagtgctg

SEQ ID NO: 101 - protein, 107 aa, Homo Sapiens
YVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVL

SEQ ID NO: 102 - DNA, 639 bp, Homo Sapiens
tacgtgctgacccagcctccttccgtgtctgttgctcctggcgagacagccagaatcacctgtggcggcgataacatcggccggaagtccgtgtactggtatcagcagaagtccggccaggctcctgtgctggtcatctactacgactccgaccggccttctggcatccctgagagattctccggctccaactccggcaataccgccacactgaccatctccagagtggaagctggcgacgaggccgactactactgccaagtgtgggacggctcctctgaccactgggttttcggcggaggcaccaagctgacagtgctgggacaacctaaggccgctccttctgtgaccctgtttcctccatcctccgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccacaagtcctactcttgccaagtgacccacgagggctccaccgtggaaaagacagtggctcctaccgagtgctcc

SEQ ID NO: 103 - protein, 213 aa, Homo Sapiens
YVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 104 - protein, 327 aa, Artificial Sequence
  IgG4 PE human heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 105 - protein, 327 aa, Artificial Sequence
  IgG4 human heavy chain constant region with knobs-into-holes mutations and hinge mutation. Type a (IgG4ra)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 327 aa, Artificial Sequence
  IgG4 human heavy chain constant region with knobs-into-holes mutations and hinge mutation. Type b (IgG4yb)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 107 - protein, 325 aa, Artificial Sequence
  IgG4 human heavy chain constant region with P (hinge) mutation and K439E
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 108 - protein, 325 aa, Artificial Sequence
  IgG4 human heavy chain constant region with P (hinge) mutation and E356K
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 109 - DNA, 975 bp, Artificial Sequence
  IgG4-P K439E encoding nucleic acid
gcttccaccaagggacccagcgttttccctctggctccttgctccagatccacctccgagtctacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacacctgtaatgtggaccacaagccttccaacaccaaggtggacaagcgcgtggaatctaagtacggccctccttgtcctccatgtcctgctccagagtttctcggcggaccctccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaatgccaagaccaagcctagagaggaacagtacaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagcctcgagaaccccaggtttacaccctgcctccaagccaagaggaaatgaccaagaaccaggtgtccctgacctgtctcgtgaagggcttctacccctccgatatcgccgtggaatgggagtctaatggccagccagagaacaactacaagaccacacctccagtgctggactccgacggctcattctttctgtactccaagctgacagtggacaagtcccggtggcaagagggcaacgtgttctcctgctctgtgatgcacgaggccctgcacaaccactacacccaagagtccctgtctctgtcccct

SEQ ID NO: 110 - DNA, 975 bp, Artificial Sequence
  IgG4-P E356K encoding nucleic acid
gcttccaccaagggacccagcgtgttccctctggctccttgctccagatccacctccgagtctacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggaacccagacctacacctgtaatgtggaccacaagccttccaacaccaaggtggacaagcgcgtggaatctaagtacggccctccttgtcctccatgtcctgctccagagtttctcggcggaccctccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagcctcgagaaccccaggtttacaccctgcctccaagccagaaagagatgaccaagaaccaggtgtccctgacctgcctcgtgaagggcttctacccttccgatatcgccgtggaatgggagagcaatggccagccagagaacaactacaagaccacacctcctgtgctggactccgatggctcattctttctgtactccaagctgacagtggacaagtcccggtggcaagagggcaacgtgttctcctgctctgtgatgcacgaggccctgcacaaccactacacccagaagtccctgtctctgtcccct

SEQ ID NO: 111 - DNA, 318 bp, Homo Sapiens
ggacaacctaaggccgctccttctgtgaccctgtttcctccatcctccgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccacaagtcctactcttgccaagtgacccacgagggctccaccgtggaaaagacagtggctcctaccgagtgctcc

SEQ ID NO: 112 - protein, 106 aa, Homo Sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 113 - protein, 108 aa, Homo Sapiens
KRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 5 aa, Homo Sapiens
TDARC

SEQ ID NO: 115 - protein, 128 aa, Homo Sapiens; identical to SEQ ID NO: 68

SEQ ID NO: 116 - protein, 110 aa, Homo Sapiens
QAGLTQPPSVSKGLRQTATLTCTGNSNNVGNLGAAWLQQHQGHPPKLLSYRNNNRPSGISERFFASRSGNTASLTITGLQPEDEADYYCSAWDSSLSAWVFGGGTKLTVL

SEQ ID NO: 117 - protein, 108 aa, Homo Sapiens
GLTQPPSVSKGLRQTATLTCTGNSNNVGNLGAAWLQQHQGHPPKLLSYRNNNRPSGISERFFASRSGNTASLTITGLQPEDEADYYCSAWDSSLSAWVFGGGTKLTVL

SEQ ID NO: 118 - DNA, 296 bp, Homo Sapiens
caggcagggctgactcagccaccctcggtgtccaagggcttgagacagaccgccacactcacctgcactgggaacagcaacaatgttggcaaccaaggagcagcttggctgcagcagcaccagggccaccctcccaaactcctatcctacaggaataacaaccggccctcagggatctcagagagattatctgcatccaggtcaggaaacacagcctccctgaccattactggactccagcctgaggacgaggctgactattactgctcagcatgggacagcagcctcagtgctca

SEQ ID NO: 119 - DNA, 296 bp, Homo Sapiens
caggcagggctgactcagccaccctcggtgtccaagggcttgagacagaccgccacactcacctgcactgggaacagcaacattgttggcaaccaaggagcagcttggctgcagcagcaccagggccaccctcccaaactcctatcctacaggaataacaaccggccctcagggatctcagagagattctctgcatccaggtcaggaaacacagcctccctgaccattactggactccagcctgaggacgaggctgactattactgctcagcattggacagcagcctcagtgctca

SEQ ID NO: 120 - DNA, 290 bp, Homo Sapiens; identical to SEQ ID NO: 58

SEQ ID NO: 121 - protein, 19 aa, Homo Sapiens
MPWALLLLTLLTHSAVSVV

SEQ ID NO: 122 - DNA, 330 bp, Homo Sapiens
caggcagggctgactcagccaccctcggtgtccaagggcttgagacagaccgccacactcacctgcactgggaacagcaacaatgttggcaacctaggagcagcttggctgcagcagcaccagggccaccctcccaaactcctatcctacaggaataataaccggccctcagggatctcagagagattctttgcatccaggtcaggaaacacagcctccctgaccattactggactccagcctgaggacgaggctgactattactgctcagcatgggacagcagcctcagtgcttgggtgttcggcggagggaccaagctgaccgtccta

SEQ ID NO: 123 - protein, 9 aa, Homo Sapiens
QAGLTQPPS

SEQ ID NO: 124 - protein, 7 aa, Homo Sapiens
GLTQPPS

(19) United States
(12) Patent Application Publication
(10) Pub. No.: US 2023/0039447 A1
(43) Pub. Date: Feb. 9, 2023

(54) EXPRESSION OF SEQUENCE-ENGINEERED LAMBDA ANTIBODY LIGHT CHAINS

(71) Applicant: KYMAB LIMITED, Cambridge (GB)

(72) Inventors: John Kenneth BLACKWOOD, Cambridge (GB); Wei WANG, Cambridge (GB); Benjamin Henry JENKINS, Farnborough Hampshire (GB)

(21) Appl. No.: 17/782,986

(22) PCT Filed: Dec. 18, 2020
(86) PCT No.: PCT/EP2020/086920

§ 371 Date: Jun. 6, 2022

(30) Foreign Application Priority Data
Dec. 20, 2019 (EP) PCT/EP2019/086808
Dec. 20, 2019 (GB) 1919077.6
May 14, 2020 (EP) PCT/EP2020/063515

(51) Int. Cl. C07K 16/36; A61P 7/02; C07K 16/40; C07K 16/46

(52) U.S. Cl. CPC C07K 16/36; A61P 7/02; C07K 16/40; C07K 16/468; C07K 2317/31; C07K 2317/515; C07K 2317/522; C07K 2317/524; C07K 2317/526; C07K 2317/565; C07K 2317/75; C07K 2317/567; C07K 2317/92; C07K 2317/94; C07K 2319/02

(57) ABSTRACT

Expression of lambda antibody light chain with non-native leader sequence, wherein light chain is sequence-engineered to restore native N terminus. Immunoglobulin lambda variable domain sequence comprising N terminal deletion for expression with non-native N terminal signal peptide.

### FIELD OF THE INVENTION

The present invention relates to antibody lambda (λ) light chain variable (VL) domains and to antibodies comprising them (λ antibodies). The invention further relates to expression of λ antibodies from engineered nucleic acid in recombinant host cells.

### BACKGROUND

Monoclonal antibodies are a successful and expanding class of biopharmaceuticals. The majority of antibodies in current clinical use are IgG immunoglobulins comprising two disulphide-linked pairs of heavy and light polypeptide chains. In natural human antibodies there are two classes of antibody light chain, kappa (κ) and lambda (λ), so an IgG may be either an IgGκ or an IgGλ, depending on whether the light chain was produced by rearrangement of the immunoglobulin κ or λ light chain locus in the developing B lymphocyte. In humans, IgGκ and IgGλ are found in approximately equal amounts (55% κ; 45% λ). However, the majority of antibodies developed for human clinical use are IgGκ antibodies, with relatively few λ antibodies being in production.

The main production method for monoclonal antibodies is recombinant expression of nucleic acid encoding the antibody heavy and light chains in cultured host cells in vitro. In a typical production system, antibody heavy and light chains are co-translated and secreted from the host cell.

Efforts have been made to increase polypeptide expression by varying many different aspects of recombinant systems, since higher antibody yields improve the efficiency of manufacture and reduce production costs.

One perceived bottleneck in the secretory pathway is translocation of the antibody polypeptides into the lumen of the endoplasmic reticulum (ER), via signal peptides. The signal peptide is a short (15-30 amino acid) sequence at the N terminus of the antibody heavy and light chains, which directs its translocation and is cleaved during the translocation process so is not a part of the secreted mature polypeptide chains. Efforts have been made to find signal peptides which generate higher antibody yields.

In addition to their role in secretion efficiency, signal peptides are linked to the industrial problem of cleavage heterogeneity which occurs as a result of non-specific cleavage of the signal peptide. This phenomenon can lead to either elongation or truncation of the N-terminus of the heavy and light chains, which may not be suitable for biopharmaceutical therapeutics and which may change the antibody affinity. Modified signal peptides have also been reported to modulate folding, thermodynamic stability and aggregation propensities of their cargo proteins. Effects of signal peptides and other variables in monoclonal antibody production were recently reviewed by Gupta et al. (Biotechnol Adv 2019).

Rance & Young (Evaluation of Alternative Signal Sequences, in T. Noll (ed.), Cells and Culture, ESACT Proceedings 4, 271 DOI 10.1007/978-90-481-3419-9_47; WO2008/148519 Lonza) evaluated 19 signal sequences with respect to antibody production in a transient expression system and from stable cell lines. The antibody genes were designed to include either the signal sequences routinely used at Lonza or 19 alternative sequences (V1-V19). They identified a number of signal sequences which reportedly provided a proper processing and an efficient secretion of operably linked polypeptide sequences when used in expression cassettes in mammalian host cells, including V12, V14, V16 and V19 shown in Table P herein. V19 and V17 were said to produce a particularly strong increase in mean antibody concentration over control cell lines employing the signal sequences routinely used.

Kotia and Raghani (Anal Biochem 399 190-195 2010) described heterogeneity in a monoclonal antibody due to varying cleavage sites of the signal peptides in both the heavy chain and light chain of a monoclonal antibody expressed in CHO cells.

Haryadi et al (PLoS ONE 10(2): e0116878 2015) optimised the signal peptide sequences for five top-selling monoclonal antibodies. Optimisation of the signal peptide for rituximab resulted in two-fold increase in titre compared with the native signal peptide.

Gibson et al (Biotechnol Bioeng 114: 1970-1977 2017) and WO2017/072310 (Medlmmune) reported that recombinant antibody light chains having a murine secretory leader sequence MGWSCIILFLVATATGVHS (SEQ ID NO: 74) a commonly used signal sequence for recombinant antibody production) and an SYE motif at the N-terminus are truncated during post-translational processing, observing that about 3-8% of the final antibody product MED18490 contained a truncated light chain. The truncated light chain was missing three amino acids at its N-terminus (SYE) and was considered a risk to product development. Two protein engineering solutions were attempted to prevent truncation of the light chain N terminus: altering the SYE amino acid sequence or changing the secretory leader peptide sequence. They exemplified two λ leader sequences, MAWTPLLLPLLTLCTSEA (Vλ3 family SEQ ID NO: 75) and MAGFPLLLTLLTHCAGSWA (Vλ1 family SEQ ID NO: 76), and a κ leader sequence MDMRVPAQLLGLLLLWLPGAKC (Vκ1 family SEQ ID NO: 77) and the SYE truncation did not occur with these leader sequences.

### SUMMARY OF THE INVENTION

The present inventors discovered that changing the signal peptide of a λ VL domain causes a change in the site at which the signal peptide is cleaved from the VL domain, so that different signal peptides result in λ VL domains having different N-terminal sequences. Antibodies containing such λ VL domains (e.g., antibodies comprising two heavy chains and two λ light chains) therefore have different VL domain N-terminal amino acid sequences, depending on the amino acid sequence of the signal peptide expressed from their encoding nucleic acid.

Unlike earlier reports of heterogeneity in expression, where antibody chains of different sequences are obtained from the same encoding nucleic acid, the present inventors observed consistent expression of a single VL domain amino acid sequence per nucleic acid coding sequence. The exact N-terminal sequence of the VL domain, however, differed depending on the upstream sequence of the signal peptide present in the pre-cleaved light chain.

Specifically, the inventors compared expression of a λ light chain comprising an N-terminal signal peptide which was either (i) the native signal peptide associated with the encoding vλ gene segment in the germline DNA or (ii) a signal peptide commonly used for light chain expression in recombinant host cells, but which originated from a different v gene segment (mouse vκ gene segment). They observed that the λ light chain expressed with the native signal peptide was cleaved to provide a mature light chain in which the N-terminal residue corresponded to IMGT position 2, whereas the λ light chain expressed with the non-native signal peptide was cleaved to provide a mature light chain in which the N-terminal residue corresponded to IMGT position 1. This is surprising, since it has generally been believed that IMGT position 1 is the first residue of the mature polypeptide chain in an antibody. The IMGT numbering of VL domains, and the VL domain sequences represented in various public sequence databases, pre-suppose that the N-terminal residue of the VL domain is IMGT position 1. The present inventors are believed to be the first to discover that there are λ antibodies in which the N-terminal residue of the VL domain corresponds to IMGT position 2 when the antibody is expressed from its native encoding nucleic acid. The work presented herein indicates that certain naturally occurring λ antibodies, and λ antibodies expressed using nucleotide sequences encoding the light chain with its native signal peptide, lack the N-terminal amino acid which was previously assumed to be present based on IMGT notation, i.e., these λ antibodies do not have an amino acid at IMGT position 1. As demonstrated in various embodiments, a native N-terminal signal peptide is cleaved to provide a λ VL domain having an N-terminal residue corresponding to IMGT position 2, whereas a non-native N-terminal signal peptide is cleaved to provide a λ VL domain having an N-terminal residue corresponding to IMGT position 1. The site of cleavage may thus differ by one amino acid depending on the sequence of the signal peptide.

Similarly, in another experiment, the inventors compared expression of another λ light chain comprising an N-terminal signal peptide which was either (i) the native signal peptide associated with the encoding vλ gene segment in the germline DNA or (ii) the “Campath leader”, which is a different, non-native signal peptide that is known in the art for use in antibody expression. Again, the inventors observed that the λ light chain expressed with the native signal peptide was cleaved to provide a mature light chain which was N-terminally truncated compared with the λ light chain expressed with the non-native signal peptide. The N terminal residue of the light chain expressed with (and cleaved from) the native signal peptide corresponded to IMGT position 3, whereas the light chain expressed with (and cleaved from) the non-native signal peptide corresponded to IMGT position 1. Expression of the same λ light chain with a mouse vκ signal peptide interestingly also resulted in cleavage to generate an N terminus of IMGT position 1, but here heterogeneous cleavage was also observed, with a smaller fraction of the light chain being cleaved to provide an N terminus of IMGT position 3. Notably, the inventors found that in some embodiments the variation in cleavage site, and thus the change in N-terminal VL domain sequence, influenced the properties of the λ antibody. For example, A antibodies comprising the VL domains with different N-terminal sequences showed different functional activity. In various embodiments, the invention builds on this effect to provide A antibodies with desired properties. For example, the influence of a non-native signal peptide sequence on the N-terminal sequence of the VL domain may be over-ridden by a “forced” deletion of IMGT position 1 when expressing a VL domain with a non-native signal peptide, so that the resulting mature VL domain has the same sequence as if it had been expressed with its native signal peptide (i.e., the N-terminal residue of the mature VL domain corresponds to IMGT position 2). As demonstrated in the Examples, this restored activity of the antibody which had been lost on changing from its native light chain leader to a non-native leader. In various aspects, the present invention provides λ antibodies, λ VL domains, encoding nucleic acids, methods and for their expression in recombinant host cells, and compositions containing λ antibodies isolated from such recombinant expression systems.

Aspects of the invention are set out in the appended claims and in the accompanying disclosure.

In a first aspect, the present invention provides a polypeptide comprising an antibody VL domain with an N-terminal signal peptide, wherein

the VL domain comprises a sequence derived from a v λ gene segment and a sequence derived from a j gene segment, wherein

the N-terminal signal peptide differs from the native N-terminal signal peptide for said vλ gene segment, and wherein

the residue corresponding to IMGT position 1 of the VL domain is absent.

The polypeptide thus lacks an amino acid at IMGT position 1 of the VL domain, so that effectively there is a deletion at this position, and the N-terminal residue of the VL domain corresponds to IMGT position 2.

In embodiments of the invention, the VL domain is one which, when expressed with its native signal peptide (i.e., the N-terminal signal peptide for the vλ gene segment from which said VL domain is derived), lacks an amino acid at IMGT position 1 of the VL domain. When expressed and cleaved from its native signal peptide, the N-terminal residue of the VL domain thus corresponds to IMGT position 2.

The v λ gene segment may be a human vλ3 gene segment, e.g., vλ3-21.

In another example, the present invention provides a polypeptide comprising an antibody VL domain with an N-terminal signal peptide, wherein

the VL domain comprises a sequence derived from a v λ gene segment and a sequence derived from a j gene segment, wherein

the N-terminal signal peptide differs from the native N-terminal signal peptide for said vλ gene segment, and wherein

the residues corresponding to IMGT positions 1 and 2 of the VL domain are absent.

The polypeptide thus lacks an amino acid at both IMGT position 1 and position 2 of the VL domain, so that effectively there is a deletion at these positions, and the N-terminal residue of the VL domain corresponds to IMGT position 3.

In embodiments of the invention, the VL domain is one which, when expressed with its native signal peptide (i.e., the N-terminal signal peptide for the vλ gene segment from which said VL domain is derived), lacks an amino acid at IMGT position 1 and at IMGT position 2 of the VL domain. When expressed and cleaved from its native signal peptide, the N-terminal residue of the VL domain thus corresponds to IMGT position 3.

The polypeptide thus lacks an amino acid at IMGT position 1 of the VL domain, so that effectively there is a deletion at this position, and the N-terminal residue of the VL domain corresponds to IMGT position 2.

The v λ gene segment may be a human vλ10 gene segment, e.g., vλ10-54.

As demonstrated herein, when a human λ VL domain is expressed and cleaved from the non-native mouse v κ signal peptide MSVPTQVLGLLLLWLTDARC (SEQ ID NO: 62), the VL domain differs at its N terminus compared with the same human λ VL domain expressed and cleaved from its native signal peptide (e.g., for a vλ3-21 derived VL domain, the native human vλ3 signal peptide, or for a vλ10-54 derived VL domain, the native human vλ10 signal peptide). To rectify this anomaly, the N terminus of the VL domain expressed from the non-native leader can be engineered to match that of the VL domain expressed from the native leader. Thus, the N-terminal truncation which is found in the VL domain expressed from its native leader (truncated relative to the product expressed with the non-native leader and/or relative to predicted N terminus at IMGT position 1) is introduced into the nucleotide sequence encoding the VL domain. For example, an initial one or two amino acid residues (IMGT position 1, and optionally also IMGT position 2) are deleted from the encoding nucleic acid.

A polypeptide according to the present invention may thus comprise a VL domain with an N-terminal signal peptide, wherein

the VL domain comprises a sequence derived from a v λ gene segment and a sequence derived from a j gene segment, wherein

the N-terminal signal peptide is the mouse κ signal peptide SEQ ID NO: 62, wherein

the VL domain comprises an N-terminal truncation relative to the amino acid sequence encoded by the v λ gene segment starting at IMGT position 1.

As discussed, the N-terminal truncation may be a deletion of the amino acid residue corresponding to IMGT position 1, so that the N terminal residue of the VL domain corresponds to IMGT position 2. Alternatively, the N-terminal truncation may be a deletion of the amino acid residue corresponding to IMGT positions 1 and 2, so that the N-terminal residue of the VL domain corresponds to IMGT position 3. These minor truncations have been observed with structurally distinct v λ gene segments, and other examples may be identified by comparing polypeptide products of expression with other native and non-native signal peptides, wherein the VL domains comprise VL domains derived from other v λ gene segments, optionally any of the human v λ gene segments exemplified herein.

A method of the invention may comprise

(i) providing a nucleotide sequence encoding an antibody light chain variable (VL) domain,

(ii) providing a first DNA molecule comprising the said nucleotide sequence preceded by a leader sequence encoding the native signal peptide,

(iii) providing a second DNA molecule comprising the said nucleotide sequence preceded by a leader sequence encoding a non-native signal peptide,

(iv) expressing the first and second DNA in host cells (e.g., CHO cells) to produce first and second polypeptides respectively comprising the VL domain,

(v) comparing VL domain amino acid sequences of the first and second polypeptides expressed from the host cells (e.g., using mass spectrometry),

(vi) identifying an N-terminal truncation in the sequence of the first polypeptide compared with the second polypeptide,

(vii) providing an engineered nucleotide sequence encoding the sequence of the first polypeptide, including the N-terminal truncation,

(viii) providing a third DNA molecule comprising the said engineered nucleotide sequence preceded by a leader sequence encoding a non-native signal peptide, and

(ix) expressing the third DNA molecule in a host cell to produce a third polypeptide comprising the encoded VL domain, wherein the amino acid sequence of the third polypeptide is identical to that of the first polypeptide.

Thus, differences in the position of cleavage between the signal peptide and the VL domain amino acid sequence are identifiable by comparing the products of expression with the native vs a non-native signal peptide. The identification of differences then informs design of an engineered VL domain sequence which, when expressed from the non-native leader, will be cleaved to produce a VL domain sequence with a natural or native N terminus, corresponding to the product of cleavage that would be obtained for VL domains expressed with the native signal peptide.

VL domains herein may be comprised in antibody light chains and/or in antibody molecules (e.g., human IgG). Thus, expression of VL domains and/or light chains herein may be in the context of expression of an antibody, and may involve co-expression of an antibody VH domain and/or antibody heavy chain in the host cell.

A VL domain is derived from recombination of a v gene segment and a j gene segment. In vivo, recombination takes place at the DNA level and involves genomic rearrangement in cells that develop into B cells. VL domains of antibodies expressed by B cells thus comprise amino acid sequences translated from nucleic acid derived from recombination of the v and j gene segment. Examination of the amino acid sequence of a VL domain allows the v and j gene segments to be identified, by comparison against the translated sequences of germline v and j gene segments.

Preferably, a gene segment herein is a vertebrate gene segment, e.g., a mammalian gene segment. More preferably, the gene segment is a human gene segment. The invention has particular relevance for expression of human VL domains and human antibodies comprising them. A human VL domain is derived from recombination of a human v gene segment and a human j gene segment. Thus, in preferred embodiments, the v λ gene segment is a human v λ gene segment and/or the j gene segment is a human j gene segment. The j gene segment is preferably a λ gene segment, although combination of a v λ with a j κ gene segment is possible.

Appended Table L shows examples of human λ v gene segments. Preferably, the v λ gene segment is a vλ3 gene segment, i.e., a member of the IGLV3 family. This includes the following human v λ gene segments: vλ3-1, vλ3-9, vλ3-10, vλ3-12, vλ3-13, vλ3-16, vλ3-19, vλ3-21, vλ3-22, vλ3-25, vλ3-27, vλ3-31 and vλ3-32. Preferably, the v λ gene segment is a functional gene segment. Thus, the v λ gene segment is optionally selected from the group consisting of: vλ3-1, vλ3-9, vλ3-10, vλ3-12, vλ3-16, vλ3-19, vλ3-21, vλ3-22, vλ3-25 and vλ3-27. Human vλ3 gene segments are especially preferred in embodiments of the invention in which expression from the native signal peptide generates a polypeptide product wherein the N-terminal residue corresponds to IMGT position 2.

In other embodiments, the human v λ gene segment is a vλ10 gene segment, i.e., a member of the IGLV10 family. Optionally, it is vλ10-54. Known functional alleles include vλ10-54*02 and vλ10-54*01.

Amino acid sequences of VL domains generated from v λ gene segment are available from IMGT and examples are shown in Table L. IMGT predicts the first residue of the mature VL domain and designates this as IMGT position 1. A v λ gene segment according to the present invention may encode an amino acid sequence in which the first residue (IMGT position 1) is Ser (S). A v λ gene segment according to the present invention may encode an amino acid sequence in which the first two residues (IMGT positions 1 and 2) are Ser and Tyr (SY). It may encode an amino acid sequence in which the first three residues (IMGT positions 1, 2 and 3) are Ser, Tyr and Val (SYV).

A preferred vλ gene segment is vλ3-21. Alleles of vλ3-21 are known. These include vλ3-21*01, vλ3-21*d01, vλ3-31*02 and vλ3-21*03.

A polypeptide according to the present invention may comprise a VL domain with an N-terminal signal peptide, wherein

the VL domain comprises a sequence derived from a human vλ3 gene segment (e.g., vλ3-21) and a sequence derived from a j gene segment, wherein

the N-terminal signal peptide is not a native signal peptide for a human vλ3 gene segment, wherein

the vλ3 gene segment encodes an amino acid sequence for which the N-terminal residues at IMGT positions 1 and 2 are Ser and Tyr (SY) respectively, and wherein

the VL domain comprises a deletion of Ser at IMGT position 1.

The VL domain comprises the signal peptide immediately fused to the VL domain, wherein the VL domain is derived from recombination of a human vλ3 gene segment and a v j gene segment, wherein the VL domain lacks a Ser residue at IMGT position 1, i.e., N-terminal Ser of the VL domain is deleted.

On expression in a host cell, the N-terminal residue of the VL domain following cleavage of the signal peptide is therefore the residue (e.g., Tyr) at IMGT position 2. Gene segment v3-21 encodes an amino acid sequence in which the first three residues according to IMGT are Ser Tyr Val (SYV) and therefore, when the Ser at IMGT position 1 is deleted, the N-terminal residues of the VL domain following cleavage of the signal peptide are Tyr Val (YV).

In embodiments of the invention, the non-native signal peptide (the N-terminal signal peptide which differs from the native N-terminal signal peptide for said vλ gene segment) is one which would be cleaved from the polypeptide immediately before the residue corresponding to IMGT position 1 of the VL domain, if said residue were present. Thus, when the residue corresponding to IMGT position 1 of the VL domain (e.g., Ser) is present in the VL domain of the polypeptide, the signal peptide is cleaved immediately before said residue corresponding to IMGT position 1 of the VL domain. Deletion of said residue thus avoids the presence of this residue in the mature polypeptide. In various embodiments this may have the effect of restoring or enhancing function of the antibody as compared with an antibody comprising the VL domain in which said residue is present.

Signal peptides may be of varying length but usually between approximately 18-22 amino acids. Optionally, the signal peptide is 20 amino acids in length. The signal peptide is not the native signal peptide for the vλ gene segment in the polypeptide. It may be a signal peptide from another gene of the same or a different species, e.g., a mouse signal peptide may be combined with a VL domain derived from recombination of human gene segments. Although the signal peptide may be naturally occurring, and may thus be the native signal peptide from a different genomic coding sequence, it is optionally not the signal peptide from any vλ gene segment. Examples of heterologous signal peptides are disclosed herein and include signal peptides from light chain κ gene segments, including mouse v κ gene segments such as the commonly used signal peptide MSVPTQVLGLLLLWLTDARC (SEQ ID NO: 62).

The C-terminal region of the signal peptide is believed to influence the site of cleavage. Preferably, the signal peptide comprises a C-terminal Cys residue and thus on expression in a host cell is cleaved from the VL domain immediately after the Cys. The N-terminal residue of the mature VL domain is thus the residue immediately following the C-terminal Cys of the signal peptide. The signal peptide may comprise a C-terminal sequence Ala Arg Cys (ARC). It may comprise a C-terminal sequence Thr Asp Ala Arg Cys (TDARC SEQ ID NO: 114).

In one embodiment, a polypeptide according to the present invention comprises a VL domain with an N-terminal signal peptide, wherein

the VL domain comprises a sequence derived from a human vλ3-31 gene segment and a sequence derived from a human j λ gene segment, wherein

the N-terminal signal peptide is a mouse v κ gene segment signal peptide, and wherein

the VL domain comprises a deletion of Ser at IMGT position 1.

The VL domain may be the 0325L VL domain exemplified herein, or it may be a variant thereof such as a VL domain having at least 90% amino acid sequence identity to the 0325 VL domain. Amino acid sequence identity may be at least 95%, at least 96%, at least 97%, at least 98% or at least 99%.

Further aspects of the present invention include nucleic acid molecules encoding the polypeptides (e.g., cDNA or genomic DNA), such as DNA vectors.

Host cells may be transfected with said nucleic acid and cultured for expression of the polypeptides. Host cells may be provided in vitro and cultured under laboratory conditions. Host cells may also be stored, e.g., as frozen stocks. Host cells may comprise recombinant DNA encoding the polypeptide, e.g., wherein the encoding DNA is stably integrated into the cellular DNA (e.g., in the host cell genome). Cells that are transiently transfected with the encoding nucleic acid may also be used. A host cell may be a eukaryotic cell, e.g., a vertebrate cell, e.g., a mammalian cell. A number of established cell lines for polypeptide expression are known in the art and are obtainable from cell banks. Such cells may also be further modified for additional desired attributes. Examples include Chinese Hamster Ovary (CHO) cells, e.g., CHO-K1 or CHO GS, and Human Embryonic Kidney (HEK) cells, e.g., HEK293.

On expression within host cells, the signal peptide directs the polypeptide to the ER membrane where it is cleaved by signal peptidase. The signal peptide is cleaved from the polypeptide to provide a mature VL domain comprising an N-terminal residue corresponding to IMGT position 2. Cells may secrete the resultant polypeptide comprising the mature VL domain (lacking the signal peptide), whereupon it may be recovered from the culture medium and optionally further purified and formulated as desired. Alternatively cells may display the polypeptide on the cell surface as a membrane protein (e.g., IgM).

Accordingly, further aspects of the invention relate to methods of producing a polypeptide comprising a VL domain, wherein the VL domain has an N-terminus generated by cleavage of an N-terminal signal polypeptide as described herein, wherein the N-terminus of the VL domain corresponds to IMGT position 2.

A method of expressing a polypeptide comprising a VL domain may comprise

culturing a population of cells under conditions for expression of the polypeptide, wherein the cells comprise nucleic acid encoding a polypeptide comprising an N-terminal signal peptide and a VL domain, wherein

the N-terminal signal peptide is cleaved off the VL domain to provide a polypeptide comprising a mature VL domain, and wherein

the N-terminal residue of the polypeptide comprising the mature VL domain is IMGT position 2 of the VL domain.

The polypeptide may be an antibody light chain, optionally comprising a λ constant domain. The method may comprise producing an antibody comprising said VL domain or light chain and a VH domain or heavy chain. Antibody light and heavy chains may be co-expressed within the same cell or separately expressed and then assembled.

One method comprises expressing an antibody comprising a VH domain and a VL domain, wherein the VL domain comprises a sequence derived from a v λ gene segment and a sequence derived from a j gene segment, including

providing cells with nucleic acid encoding a VL domain and N-terminal signal peptide as described herein, wherein the cells further comprise nucleic acid encoding the VH domain,

culturing said population of cells under conditions for expression of the polypeptide comprising the VL domain and for expression of the VH domain, wherein

the N-terminal signal peptide is cleaved off the VL domain to provide a polypeptide comprising a mature VL domain, wherein

the N-terminal residue of the polypeptide comprising the mature VL domain is IMGT position 2 of the VL domain, and wherein

the VL domain assembles with the VH domain to provide said antibody.

Thus, in various embodiments herein, a cell comprises nucleic acid encoding the polypeptide comprising the N-terminal signal peptide and the VL domain (e.g., an antibody light chain), and further comprises nucleic acid encoding an antibody VH domain (e.g., an antibody heavy chain). Optionally, a cell comprises nucleic acid encoding two different antibody VH domains, e.g., it may comprise nucleic acid encoding a first antibody heavy chain and nucleic acid encoding a second antibody heavy chain, wherein both the first and second antibody heavy chain assemble with the antibody light chain. The resulting antibody may thus comprise a first binding site formed by the VH domain of the first heavy chain and the VL domain, and a second binding site formed by the VH domain of the second heavy chain and the VL domain. The first and second binding sites may bind first and second epitopes respectively, e.g., on first and second antigens. Such a cell would generate a bispecific antibody having a common light chain. Alternatively a cell may comprise nucleic acid encoding multiple different VH domains and multiple different VL domains, e.g., two different VH domains and two different VL domains, to generate a bispecific antibody having four different antibody chains.

In preferred embodiments of the invention generally, the VL domain is derived from a vλ3-21 gene segment and a jλ gene segment. The VL domain amino acid sequence may be the 0325L VL domain amino acid sequence shown herein. The polypeptide is preferably an antibody light chain comprising the 0325 VL domain, e.g., it may be the 0325L light chain shown herein. Preferred embodiments include antibodies that comprise a λ light chain comprising the VL domain, paired with a heavy chain. A bispecific antibody may comprise two different antibody heavy chains each paired with a light chain, wherein one or both light chains comprise a λ VL domain as described herein. Preferred embodiments are the bispecific antibodies, methods for their production and cells which comprise nucleic acid encoding them, wherein the bispecific antibody comprises

a first heavy chain comprising the N1280H VH domain or the N1441H VH domain, paired with a light chain comprising the 0325L VL domain; and

a second heavy chain comprising the T0999H VH domain paired with a light chain comprising the 0325L VL domain.

The first and/or second heavy chain may comprise the VH domain and a human IgG4 constant region shown in Table S. The light chain may be a common light chain, e.g., the 0325L light chain shown in Table S. Examples of full heavy and light chains are shown in Table S.

It is shown herein that deletion of the Ser at IMGT position 1 of the 0325L light chain increases the functional activity of bispecific antibodies comprising 0325L as a common light chain, compared with bispecific antibodies that include the Ser at IMGT position 1 but are otherwise identical. Without being bound by theory, the inventors believe that this may be the result of the N-terminal Ser influencing the association between the VH and VL domain. In the absence of the N-terminal Ser, the VH and VL domain of one or both arms of the bispecific antibody may bind differently to its antigen, thereby influencing the function of the antibody. Optimisation of biophysical properties is critical for the developability of therapeutic antibodies, and the IXAX bispecific antibodies described herein may exhibit greater stability in the absence of an N-terminal Ser on the light chain. Another possibility, especially relevant for bispecific antibodies, is that the presence or absence of the residue at IMGT position 1 of the light chain may affect heterodimerisation, i.e., assembly of two different heavy chains to produce the bispecific rather than assembly of two identical heavy chains to produce unwanted monospecific homodimers.

While in some embodiments an antibody may benefit from the absence of the N-terminal Ser at IMGT position 1 of the VL domain, it is possible that the desired function of other A antibodies may be improved by engineering the sequence so that the N-terminal residue at IMGT 1 is present. Depending on the intended use of the antibody, it may for example be desirable to have different antigen-binding characteristics such as a higher affinity or a lower affinity, different binding kinetics or to influence assembly of the antibody heavy and light chains. Particularly in the case of antibodies that are selected for function with an N-terminal residue present at IMGT position 1 (e.g., in the case of λ antibodies expressed with a non-native signal peptide), one may wish to ensure that said residue is retained when the antibody is recombinantly expressed in a host cell, even if this does not reflect the native structure of a naturally occurring λ antibody. It may therefore be desirable to provide a polypeptide comprising a VL domain with an N-terminal signal peptide, wherein the VL domain comprises a sequence derived from a v λ gene segment and a sequence derived from a j gene segment, wherein the N-terminal signal peptide differs from the native N-terminal signal peptide for said λ v gene segment, and wherein the residue corresponding to IMGT position 1 of the VL domain is present. An example is a polypeptide comprising the 0128L VL domain with a non-native signal peptide, e.g., the mouse κ light chain signal peptide MSVPTQVLGLLLLWLTDARC (SEQ ID NO: 62).

Further aspects of the invention thus relate to the provision of VL domains in which an N-terminal residue is present at IMGT position 1, wherein said residue is absent from the mature VL domain expressed with its native signal peptide. For example, a VL domain may be provided which comprises a sequence derived from a v λ gene segment and a sequence derived from a j gene segment, wherein the residue corresponding to IMGT position 1 of the VL domain is present. The VL domain may be one which, when expressed with the native signal peptide of its v λ gene segment, lacks a residue at IMGT position 1 and thus has an N-terminal residue corresponding to IMGT position 2. By recombinantly expressing the VL domain with a non-native signal peptide, cleavage may occur immediately before the N-terminal residue corresponding to IMGT position 1, thus changing the VL domain sequence to include the N-terminal residue at IMGT position 1. In various embodiments, the invention provides polypeptides and antibodies comprising such VL domains, nucleic acids encoding them, host cells comprising such nucleic acids and methods of production by expression from host cells.

The invention further extends to methods of altering the stability, assembly and/or antigen-binding kinetics of an antibody comprising a VH domain and a VL domain, wherein the VL domain comprises a sequence derived from a v λ gene segment and a sequence derived from a j gene segment.

Such a method may comprise providing nucleic acid encoding said antibody, comprising a nucleotide sequence encoding said VL domain with an N-terminal signal peptide, wherein the codon for the residue corresponding to IMGT position 1 of said VL domain is deleted and wherein the N-terminal signal peptide is not the native N-terminal signal peptide for said A v gene segment, and nucleic acid encoding the VH domain,

expressing said nucleic acid to provide an antibody comprising the VH domain and the VL domain, wherein the N-terminal residue of the VL domain is the residue corresponding to IMGT position 2,

wherein the stability, assembly and/or antigen-binding kinetics of the antibody are different from the stability and/or antigen-binding kinetics of an antibody comprising a VH domain and a VL domain wherein the N-terminal residue of the VL domain is the residue corresponding to IMGT position 1. Thus, stability is increased, or assembly or antigen-binding kinetics are altered, relative to an antibody expressed from nucleic acid in which the codon for IMGT residue 1 is not deleted. An influence on antibody assembly refers to pairing of the polypeptide chains or domains from which the antibody is composed. For example, when two different heavy chains and a common light chain are co-expressed, an effect may be observed on heterodimer formation, wherein the proportion of heterodimer relative to homodimers is altered.

Alternatively, a method may comprise providing nucleic acid encoding said antibody, comprising a nucleotide sequence encoding said VL domain with an N-terminal signal peptide, wherein the codon for the residue corresponding to IMGT position 1 of said VL domain is present, and wherein the N-terminal signal peptide is not the native N-terminal signal peptide for said λ v gene segment, and nucleic acid encoding the VH domain,

expressing said nucleic acid to provide an antibody comprising the VH domain and the VL domain, wherein the N-terminal residue of the VL domain is the residue corresponding to IMGT position 1,

wherein the stability and/or antigen-binding kinetics of the antibody are different from the stability, assembly and/or antigen-binding kinetics of an antibody comprising a VH domain and a VL domain wherein the N-terminal residue of the VL domain is the residue corresponding to IMGT position 2.

Alternatively, a method may comprise providing nucleic acid encoding said antibody, comprising a nucleotide sequence encoding said VL domain with an N-terminal signal peptide, wherein the codon for the residue corresponding to IMGT position 1 of said VL domain is present, and wherein the N-terminal signal peptide is the native N-terminal signal peptide for said λ v gene segment, and nucleic acid encoding the VH domain,

expressing said nucleic acid to provide an antibody comprising the VH domain and the VL domain, wherein the N-terminal residue of the VL domain is the residue corresponding to IMGT position 2,

wherein the stability, assembly and/or antigen-binding kinetics of the antibody are different from the stability and/or antigen-binding kinetics of an antibody comprising a VH domain and a VL domain wherein the N-terminal residue of the VL domain is the residue corresponding to IMGT position 1.

Thus, the sequence of the signal peptide and/or the N terminus of the VL domain can be selected or engineered to achieve cleavage at the desired location, providing either a VL domain starting at IMGT position 1 or a VL domain starting at IMGT position 2.

Equivalents: Those skilled in the art will recognise, or be able to ascertain using no more than routine experimentation, many equivalents to the specific embodiments of the invention described herein. Such equivalents are intended to be within the scope of protection of the appended claims.

### BRIEF DESCRIPTION OF THE DRAWINGS

Embodiments of the invention will now be described in detail, with reference to the drawings, in which

FIG. 1 shows:

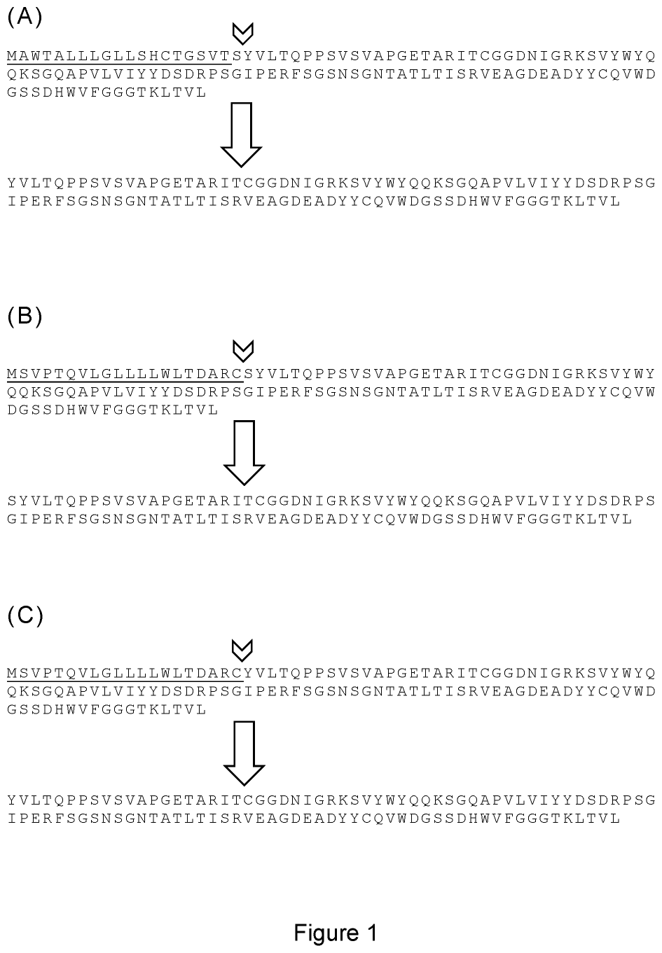

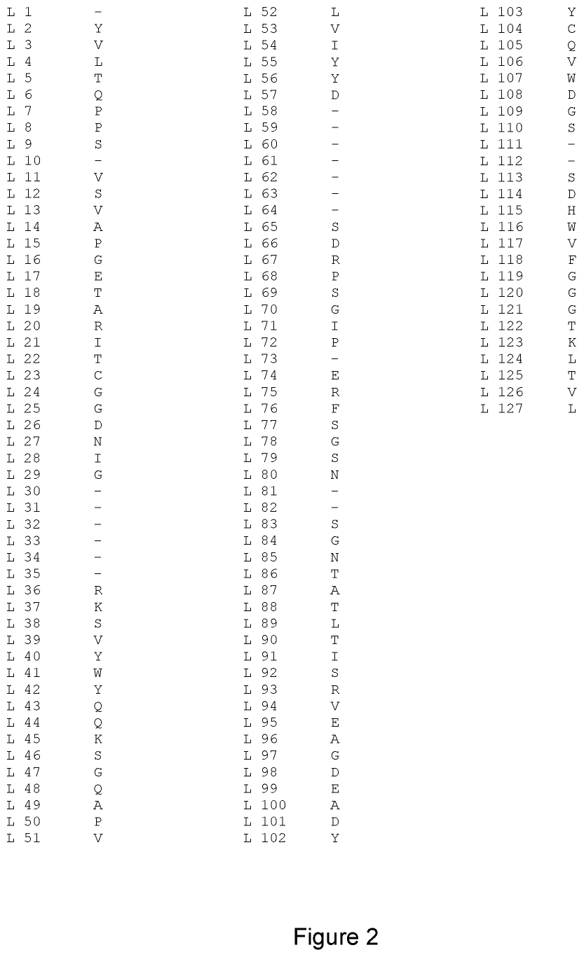

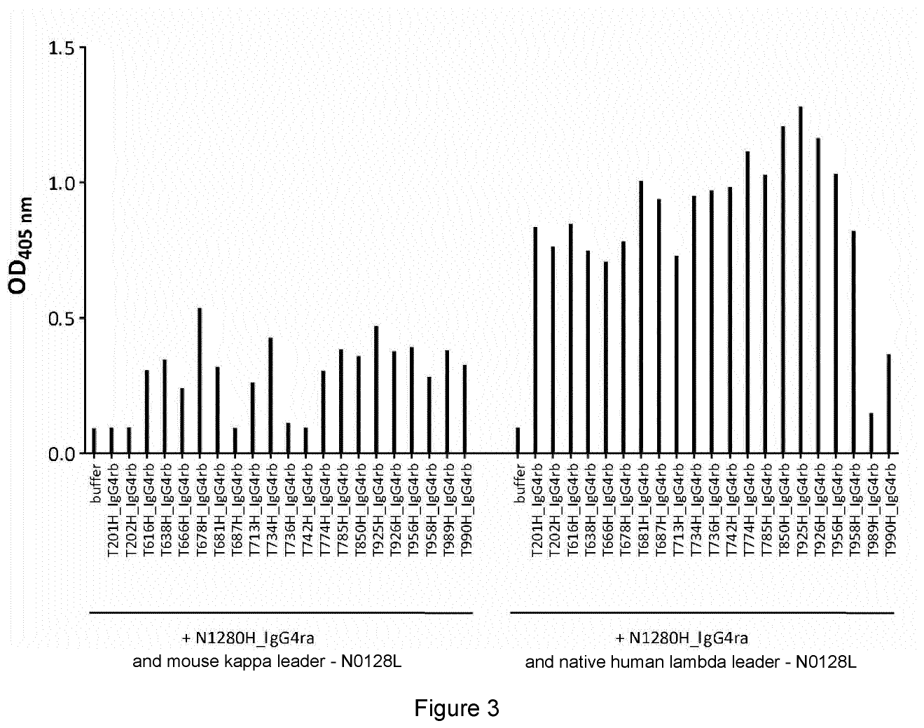

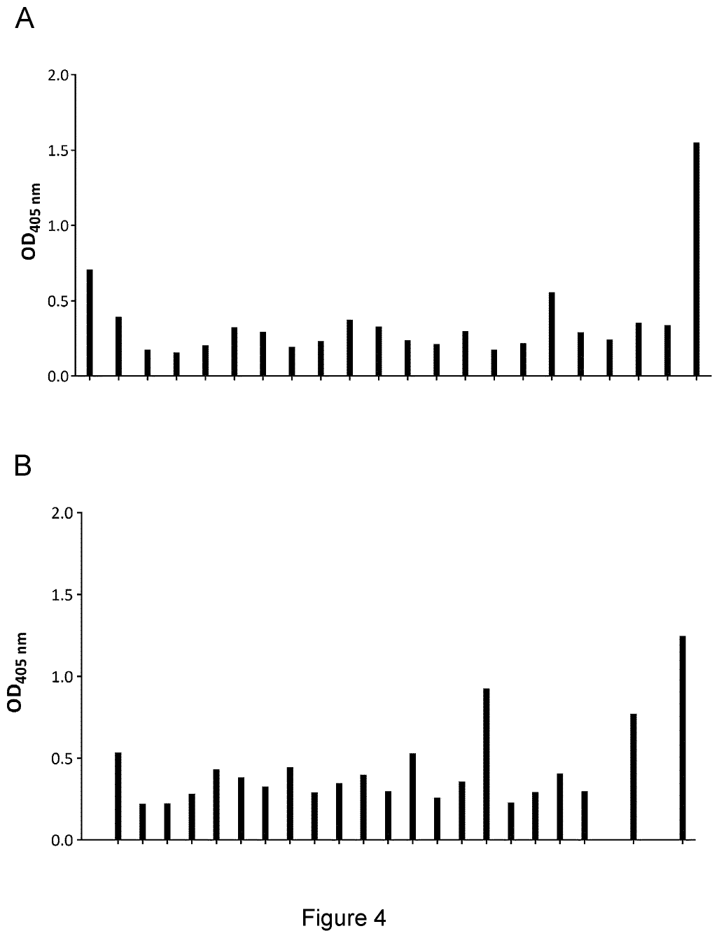

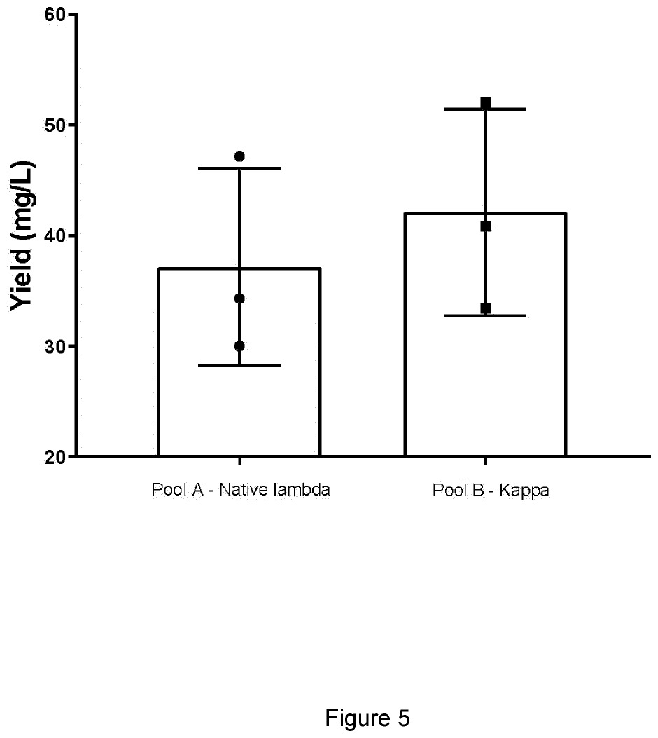

(A) Processing of the amino acid sequence of a polypeptide SEQ ID NO: 115 comprising a VL domain SEQ ID NO: 97 with native N-terminal signal peptide SEQ ID NO: 60. The native N-terminal signal peptide is underlined. The remainder of the polypeptide is the VL domain amino acid sequence as denoted by IMGT. The N-terminal signal peptide is cleaved immediately after the position corresponding to IMGT residue 1 of the VL domain. Cleavage site is marked by chevron and is between IMGT residues 1 and 2. Cleavage generates a mature VL domain SEQ ID NO: 101 in which the N-terminal residue corresponds to IMGT position 2. The generated VL domain has the amino acid sequence of the 0325 VL domain.

(B) Processing of the amino acid sequence of a polypeptide SEQ ID NO: 68 comprising a VL domain SEQ ID NO: 97 with non-native N-terminal signal peptide SEQ ID NO: 62. The non-native signal peptide is underlined. The remainder of the polypeptide is the VL domain amino acid sequence as denoted by IMGT. The N-terminal signal peptide is cleaved immediately before the position corresponding to IMGT residue 1 of the VL domain. Cleavage site is marked by chevron and is between IMGT residues −1 and 1. Cleavage generates a mature VL domain SEQ ID NO: 97 in which the N-terminal residue corresponds to IMGT position 1. The generated VL domain has the amino acid sequence of the 0128L VL domain.

(C) Processing of the amino acid sequence of a polypeptide SEQ ID NO: 66 comprising a VL domain SEQ ID NO: 101 with non-native N-terminal signal peptide SEQ ID NO: 62, wherein the residue corresponding to IMGT position 1 is deleted. The non-native signal peptide is underlined. The native N-terminal signal peptide is underlined. The remainder of the polypeptide is the VL domain amino acid sequence as denoted by IMGT except that there is no amino acid present at IMGT position 1. The N-terminal signal peptide is cleaved immediately before the position corresponding to IMGT residue 2 of the VL domain. Cleavage site is marked by chevron. Cleavage generates a mature VL domain SEQ ID NO: 101 in which the N-terminal residue corresponds to IMGT position 2. The amino acid sequence of this mature VL domain is identical to the amino acid sequence of the mature VL domain generated in (A), i.e., the 0325L VL domain.

FIG. 2 shows the amino acid sequence of VL domain 0325L. IMGT numbering is shown alongside. The 0325L VL domain is derived from recombination of human vλ gene segment IGLV3-21*d01 and human jλ gene segment IGLJ2*01.

FIG. 3 shows FXase activities for bispecific antibodies in the FXase assay, measured at 500 seconds. IXAX bispecific antibodies were expressed from nucleic acid encoding anti-FIX N1280H heavy chain, anti-FX heavy chain comprising the specified VH domain, and 0128L common light chain with non-native mouse κ leader (left) or human vλ3-21 leader (right). Human vλ3-21 leader is native to 0128L which is derived from recombination of human vλ gene segment IGLV3-21*d01 and human jλ gene segment IGLJ2*01.

FIG. 4 shows FXase activities for bispecific antibodies in the FXase assay, measured at (A) 570 seconds and (B) 600 seconds. In (A), data shown are for bispecific antibodies with heavy chains comprising N1280H VH domain and T0736H VH domain plus light chains comprising the following VL domains (left to right): N0128L from expression with native λ leader; N0310L; N0311L; N0312L; N0313L; N0314L; N0315L; N0316L; N0317L; N0318L; N0319; N0320L; N0321L; N0322L; N0323L; N0324L; N0325L; N0326L; N0327L; N0328L, N0329L; emicizumab positive control. All of N310L-N329L are from expression with codon-optimised mouse kappa leader. In (B), data shown are for bispecific antibodies with heavy chains comprising N1280H VH domain and T0736H VH domain plus light chains comprising the following VL domains (left to right): N0310L; N0311L; N0312L; N0313L; N0314L; N0315L; N0316L; N0317L; N0318L; N0319; N0320L; N0321L; N0322L; N0323L; N0324L; N0325L; N0326L; N0327L; N0328L, N0329L; N0128L from expression with native λ leader; emicizumab positive control. All of N310L-N329L are from expression with codon-optimised mouse kappa leader.

FIG. 5 shows a comparison of yield from bispecific antibodies expressed with a common light chain sequence comprising 0128L VL domain and a native lambda signal peptide (Pool A, left) or a non-native mouse kappa signal peptide (Pool B, right). Codon-optimised leader nucleotide sequences were used. Heavy chain sequences are heavy chain comprising N1172H VH domain and heavy chain comprising T0201H VH domain. Antibodies were expressed in CHO cells selected using 75 μM MSX and data are obtained from 35 ml samples after protein A purification post-dialysis into PBS.

Tables are presented at the end of the description.

### DETAILED DESCRIPTION

### Signal Peptide

Many eukaryotic polypeptides are expressed with an N-terminal sequence which directs the nascent polypeptide to the membrane of the endoplasmic reticulum (ER). This N-terminal sequence is typically referred to as a “signal peptide” or “leader”. At the ER membrane, the N-terminal sequence is cleaved by signal peptidase and the polypeptide lacking the N-terminal sequence is translocated through the membrane and will either be secreted from the cell or displayed as a membrane protein on the cell surface. The expressed polypeptide, lacking the signal peptide, may be referred to as a mature polypeptide.

Where a polypeptide has a signal peptide, cleavage of the signal peptide ordinarily creates the new N-terminus of the mature polypeptide. Thus, the amino acid residue immediately following the cleavage site becomes the N-terminal residue of the mature polypeptide. Further modification of the amino acid sequence may occur with some polypeptides, so that the mature polypeptide comprises further alterations, but this does not generally occur with antibody light chains. Thus, the polypeptide sequence lacking the signal peptide represents the mature polypeptide which is expressed by the cell. A polypeptide may thus comprise a signal peptide which is directly fused to a VL domain, wherein the signal peptide represents the N-terminal sequence of the polypeptide. The VL domain may represent the remainder of the polypeptide, or further residues and/or further domains may be included C-terminal to the VL domain. For example the polypeptide may be an antibody light chain comprising a CL domain.

At the genomic level, the signal peptide is encoded within the gene for the polypeptide. While signal peptides of different polypeptides share some common structural characteristics, they are not all identical in sequence. In the case of the variable region gene segments from which antibody variable domains are derived, each variable region gene segment of the genome has its own signal peptide. The signal peptide of a variable region gene segment in genomic DNA may be referred to as the native or germline signal peptide for that gene segment.

For recombinant gene expression, where a coding sequence is introduced into a host cell in vitro, it is common to replace the native signal peptide. The coding sequence for the mature polypeptide is thus fused to a signal peptide which is different from the native signal peptide, although it may optionally be from the same polypeptide family, e.g., in the case of an antibody VL domain it may be a signal peptide for a different v gene segment. Although the non-native signal peptide has a different amino acid sequence from the native signal peptide, the person skilled in the art expects the sequence of the mature polypeptide to be identical regardless of which signal peptide is used, since the signal peptide is cleaved from the mature polypeptide. As taught herein, in fact differences in signal peptide sequence can result in differences in mature VL domains when expressing λ antibodies. Embodiments of the present invention use a non-native or heterologous signal peptide comprising a nucleotide sequence which differs from that of the native or germline signal peptide of the vλ gene segment. The signal peptide is optionally not a v λ signal peptide.

Examples of signal peptides are shown in appended Table P. MAWTALLLGLLSHCTGSVT (SEQ ID NO: 60) is the native signal peptide for the v gene segment vλ3-21. All other signal peptide amino acid sequences shown in Table P represent examples of non-native signal peptides for a VL domain derived from vλ3-21. Codon-optimised variants of encoding nucleotide sequences may be used. Thus a coding sequence for a VL domain may be fused to an upstream nucleotide sequence encoding the non-native leader. A preferred signal peptide is MSVPTQVLGLLLLWLTDARC (SEQ ID NO: 62). For CHO cell expression, a preferred nucleotide sequence encoding this signal peptide is ATGTCTGTGCCTACACAGGTTCTGGGACTGCTGCTGCTGTGGCTGACCGACGCCAGATGT (SEQ ID NO: 64), which is codon-optimised for expression in CHO cells.

### Antibodies

Antibodies are immunoglobulins or molecules comprising immunoglobulin domains. Antibodies may be IgG, IgM, IgA, IgD or IgE molecules or molecules including antigen-specific antibody fragments thereof. The term “antibody” covers any polypeptide or protein comprising an antibody antigen-binding site. An antibody antigen-binding site (paratope) is the part of an antibody that binds to and is complementary to the epitope of its target antigen. The term “epitope” refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulphonyl groups, and, in certain embodiments, may have specific three-dimensional structural characteristics, and/or specific charge characteristics.

An antibody antigen-binding site is provided by a set of complementarity determining regions (CDRs) in an antibody VH and/or VL domain, and is capable of binding the antigen. In an example, the antibody binding site is provided by a single variable domain, e.g., a heavy chain variable domain (VH domain) or a light chain variable domain (VL domain). In another example, the binding site is provided by a VH/VL pair (an Fv) or two or more such pairs.

The antibody variable domains are the portions of the light and heavy chains of antibodies that include amino acid sequences of complementarity determining regions (CDRs; ie., CDR1, CDR2, and CDR3), and framework regions (FRs). Thus, within each of the VH and VL domains are CDRs and FRs. A VH domain comprises a set of HCDRs, and a VL domain comprises a set of LCDRs. VH refers to the variable domain of the heavy chain. VL refers to the variable domain of the light chain. Each VH and VL is typically composed of three CDRs and four FRs, arranged from amino-terminus to carboxy-terminus in the following order: FR1, CDR1, FR2, CDR2, FR3, CDR3, FR4. Amino acid positions assigned to CDRs and FRs may be defined according to IMGT nomenclature. An antibody may comprise an antibody VH domain comprising a VH CDR1, CDR2 and CDR3 and a framework. It may alternatively or also comprise an antibody VL domain comprising a VL CDR1, CDR2 and CDR3 and a framework. Example sequences of antibody VH and VL domains and CDRs form part of the present disclosure. The CDRs are defined according to the IMGT system.

An antibody may comprise one or more CDRs, e.g. a set of CDRs, within an antibody framework. The framework regions may be of human germline gene segment sequences. Thus, the antibody may be a human antibody having a VH domain comprising a set of HCDRs in a human germline framework and a VL domain comprising a set of LCDRs, e.g. in a human germline framework.

An antibody “gene segment”, e.g., a VH gene segment, D gene segment, or JH gene segment refers to oligonucleotide having a nucleic acid sequence from which that portion of an antibody is derived, e.g., a VH gene segment is an oligonucleotide comprising a nucleic acid sequence that corresponds to a polypeptide VH domain from FR1 to part of CDR3. Human v, d and j gene segments recombine to generate the VH domain, and human v and j segments recombine to generate the VL domain. The D domain or region refers to the diversity domain or region of an antibody chain. J domain or region refers to the joining domain or region of an antibody chain. Recombination of germline v d and j gene segments at an immunoglobulin heavy chain locus in the genomic DNA of a developing B cell generates DNA encoding the heavy chain. Recombination of germline vκ and jκ gene segments at the immunoglobulin κ light chain locus in the genomic DNA of a developing B cell generates DNA encoding a κ light chain. If recombination at the κ locus fails to generate a productive light chain, then the λ locus rearranges. Recombination of germline vλ and jλ gene segments at the immunoglobulin λ light chain locus in the genomic DNA of the developing B cell generates DNA encoding a λ light chain. The heavy chain assembles with either the κ or λ chain to generate an antibody.

Somatic hypermutation may result in an antibody VH or VL domain having framework regions that do not exactly match or align with the corresponding germline gene segments, but sequence alignment can be used to identify the closest gene segments and thus identify from which particular combination of gene segments a particular VH or VL domain is derived. When aligning antibody sequences with gene segments, the antibody amino acid sequence may be aligned with the amino acid sequence encoded by the gene segment, or the antibody nucleotide sequence may be aligned directly with the nucleotide sequence of the gene segment.

Sequences from human λ v gene segments are provided in Table L and Table N herein for reference.

An antibody may be a whole immunoglobulin, including constant regions, or may be an antibody fragment. An antibody fragment is a portion of an intact antibody, for example comprising the antigen binding and/or variable region of the intact antibody. The antibody fragment may include one or more constant region domains.

An antibody of the invention may be a human antibody or a chimaeric antibody comprising human variable regions and non-human (e.g., mouse) constant regions. The antibody of the invention for example has human variable regions, and optionally also has human constant regions.

Thus, antibodies optionally include constant regions or parts thereof, e.g., human antibody constant regions or parts thereof, such as a human IgG4 constant region. For example, a VL domain may be attached at its C-terminal end to antibody light chain kappa or lambda constant domains. Similarly, an antibody VH domain may be attached at its C-terminal end to all or part (e.g. a CH1 domain or Fc region) of an immunoglobulin heavy chain constant region derived from any antibody isotype, e.g. IgG, IgA, IgE and IgM and any of the isotype sub-classes, such as IgG1 or IgG4. Examples of antibody constant regions are shown in Table S.

Digestion of whole (bivalent) immunoglobulins with the enzyme papain results in two identical (monovalent) antigen-binding fragments known as “Fab” fragments, and an “Fc” fragment. The Fc has no antigen-binding activity but has the ability to crystallize. “Fab” when used herein refers to a fragment of an antibody that includes one constant and one variable domain of each of the heavy and light chains. The term “Fc region” herein is used to define a C-terminal region of an immunoglobulin heavy chain, including native-sequence Fc regions and variant Fc regions. The “Fc fragment” refers to the carboxy-terminal portions of both H chains held together by disulphides.

Digestion of antibodies with the enzyme pepsin results in a bivalent F(ab′)2 fragment in which the two arms of the antibody molecule remain linked. The F(ab′)2 fragment is a bivalent fragment including two Fab fragments linked by a disulphide bridge at the hinge region. Single-chain antibodies (e.g., scFv) are another fragment. Two different monovalent monospecific antibody fragments such as scFv may be linked together to form a bivalent bispecific antibody.

“Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognise and bind antigen, although usually at a lower affinity than the entire binding site.

An antibody may be a multispecific antibody, e.g., it may be a bispecific antibody comprising antigen-binding domains for two different antigens, wherein both antigen binding domains are formed by a VH/VL pair. Example multispecific antibody formats include FIT-Ig (see WO2015/103072), mAb-dAb, dock and lock, Fab-arm exchange, SEEDbody, Triomab, LUZ-Y, Fcab, Kλ-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, scFv-CH3 KIH, scFv-CH-CL-scFv, F(ab′)2-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv and scFv4-Ig.

In one embodiment, the bispecific antibody is a bispecific IgG comprising a first antigen-binding polypeptide arm and a second antigen-binding polypeptide arm, each polypeptide arm comprising a heavy chain and a light chain. The IgG is a tetrameric immunoglobulin comprising

a first pair of antibody heavy and light chains (heavy-light chain pair) comprising a first antigen-binding Fv region,

a second heavy-light chain pair comprising a second antigen-binding Fv region,

wherein each heavy chain comprises a VH domain and a constant region, and each light chain comprises a VL domain and a constant region, and wherein the first and second heavy-light chain pairs associate through heterodimerisation of their heavy chain constant regions to form the immunoglobulin tetramer.

Optionally, the heavy chain constant region of the first heavy-light chain pair comprises a different amino acid sequence from the heavy chain constant region of the second heavy-light chain pair, wherein the different amino acid sequences are engineered to promote heterodimerisation of the heavy chain constant regions (e.g., they may comprise knobs-into-holes mutations or charge pair mutations). In one embodiment, the heavy chain constant region of one (e.g., the first) heavy-light chain pair is a human IgG4 constant region comprising substitution K439E and wherein the heavy chain constant region of the other (e.g., the second) heavy-light chain pair is an IgG4 region comprising substitution E356K, wherein constant region numbering is according to the EU numbering system. Examples of antibody constant regions and antibody heavy chains comprising them are provided in Table S.

Optionally, the two polypeptide arms comprise a common light chain, so the light chain of the first and second heavy-light chain pairs has an identical amino acid sequence Alternatively the two polypeptide arms may comprise different light chains.

Optionally a bispecific antibody is monovalent for binding each antigen.

Examples of bispecific antibodies include antibodies that bind coagulation factor IX and factor X. Nomenclature of bispecific antibodies which have a common light chain is IXAX-nnnn.tttt.llll, wherein nnnn is a 4 digit numerical identifier of the anti-FIX VH domain, tttt is a 4 digit identifier of the anti-FX VH domain, and is a 4 digit numerical identifier of the common VL domain. Sequences are shown in appended Table S and elsewhere herein.

Preferably, a common light chain comprises the 0128L VL domain or the 0325L VL domain. It may be the 0128L light chain sequence or the 0325L light chain sequence shown in Table S.

A bispecific antibody may comprise a FIX-binding arm comprising a heavy chain comprising the N0128H VH domain (e.g., the N0128H heavy chain). Alternatively, a bispecific antibody may comprise a FIX-binding arm comprising a heavy chain comprising the N1441H VH domain, the N1442H VH domain, the N1454H VH domain or the N1172H VH domain.

A bispecific antibody may comprise a FX-binding arm comprising a heavy chain comprising the T0201H VH domain (e.g., the T0201H heavy chain). Alternatively. a bispecific antibody may comprise a FX-binding arm comprising a heavy chain comprising the T0999H VH domain or the T0736H VH domain.

Preferred embodiments of bispecific antibodies are the following IgG:

IXAX-1280.0999.0325 (anti-FIXa VH domain N1280H; anti-FX VH domain T0999H; 0325L common VL domain)

IXAX-1441.0999.0325 (anti-FIXa VH domain N1441H; anti-FX VH domain T0999H; 0325L common VL domain).

The first heavy chain may be the N1280H heavy chain or the N1441H heavy chain. The second heavy chain may be the T0999H heavy chain. The light chain may be the 0325L λ light chain.

A bispecific antibody that binds FIXa and FX and catalyses FIXa-mediated activation of FX may comprise two immunoglobulin heavy-light chain pairs, wherein

a first heavy-light chain pair comprises a FIXa binding Fv region comprising a first VH domain paired with a first VL domain, and

a second heavy-light chain pair comprises a FX binding Fv region comprising a second VH domain paired with a second VL domain, wherein

the first VH domain has at least 95% amino acid sequence identity with the N1280H VH domain,

the second VH domain has at least 95% amino acid sequence identity with the T0201H VH domain, and

the first VL domain and the second VL domain each have at least 95% amino acid sequence identity with the 0325L VL domain.

A bispecific antibody that binds FIXa and FX and catalyses FIXa-mediated activation of FX may comprise two immunoglobulin heavy-light chain pairs, wherein

a first heavy-light chain pair comprises a FIXa binding Fv region comprising a first VH domain paired with a first VL domain, wherein the first VH domain is the N1280H VH domain, and

a second heavy-light chain pair comprises a FX binding Fv region comprising a second VH domain paired with a second VL domain, wherein the second VH domain is the T0999H VH domain, and wherein

the first and second heavy-light chain pairs each comprise a common light chain comprising the 0325L VL domain.

A bispecific antibody that binds FIXa and FX and catalyses FIXa-mediated activation of FX may comprise two immunoglobulin heavy-light chain pairs, wherein

a first heavy-light chain pair comprises a FIXa binding Fv region comprising a first VH domain paired with a first VL domain, wherein the first VH domain is the N1441H VH domain, and

a second heavy-light chain pair comprises a FX binding Fv region comprising a second VH domain paired with a second VL domain, wherein the second VH domain is the T0999H VH domain, and wherein

the first and second heavy-light chain pairs each comprise a common light chain comprising the 0325L VL domain.

### IMGT Numbering

The IMGT system is described in Lefranc, M.-P. et al., Dev. Comp. Immunol., 27, 55-77 2003. Unless otherwise indicated, numbering of antibody polypeptides used herein is IMGT numbering and structural definitions antibody VH domains, VL domains, CDRs and FRs are according to IMGT. Table L shows VL domain amino acid sequences translated from encoding vλ gene segments with reference to the IMGT system. FIG. 2 shows IMGT numbering for λ VL domain 0325L.

### Encoding Nucleic Acids and Methods of Expression

Isolated nucleic acid may be provided, encoding polypeptides comprising antibody VL domains and light chains, and antibodies comprising them, according to the present invention. Nucleic acid may be DNA and/or RNA. Genomic DNA, cDNA, mRNA or other RNA, of synthetic origin, or any combination thereof can encode an antibody.

The present invention provides constructs in the form of plasmids, vectors, transcription or expression cassettes which comprise at least one polynucleotide as above. Exemplary nucleotide sequences are shown herein. Reference to a nucleotide sequence as set out herein encompasses a DNA molecule with the specified sequence, and encompasses an RNA molecule with the specified sequence in which U is substituted for T, unless context requires otherwise.

The present invention also provides a recombinant host cell that comprises one or more nucleic acids encoding the polypeptide or antibody. Methods of producing the encoded molecule may comprise expression from the nucleic acid, e.g., by culturing recombinant host cells containing the nucleic acid. The polypeptide or antibody may thus be obtained, and may be isolated and/or purified using any suitable technique, then used as appropriate. A method of production may comprise formulating the product into a composition including at least one additional component, such as a pharmaceutically acceptable excipient.

Systems for cloning and expression of a polypeptide in a variety of different host cells are well known. Suitable host cells include bacteria, mammalian cells, plant cells, filamentous fungi, yeast and baculovirus systems and transgenic plants and animals.

The expression of antibodies and antibody fragments in prokaryotic cells is well established in the art. A common bacterial host is E. coli. Expression in eukaryotic cells in culture is also available to those skilled in the art as an option for production. Mammalian cell lines available in the art for expression of a heterologous polypeptide include Chinese hamster ovary (CHO) cells, HeLa cells, baby hamster kidney cells, NSO mouse melanoma cells, YB2/0 rat myeloma cells, human embryonic kidney (HEK) cells, human embryonic retina cells and many others. CHO, NS0, Sp2/0, HEK293 and PERC6 are commonly used (Dumont et al., Crit Rev Biotechnol 36(6): 1110-1122 2016).

A popular system for recombinant expression of biopharmaceuticals is Lonza's GS System™. Glutamine synthetase (GS) is the enzyme responsible for the biosynthesis of glutamine from glutamate and ammonia. In the absence of glutamine in the growth medium, the GS enzyme is essential for the survival of mammalian cells in culture. Some mammalian cell lines, such as mouse myeloma lines, do not express sufficient GS to survive without added glutamine. With these cell lines, a transfected GS gene can function as a selectable marker by permitting growth in a glutamine-free medium. Other cell lines, such as CHO cell lines, express sufficient GS to survive without exogenous glutamine. In these cases, the GS inhibitor, methionine sulphoximine (MSX), can be used to inhibit endogenous GS activity such that only transfectants with additional GS activity can survive. Lonza supplies vectors comprising nucleic acid encoding GS and a cloning site into which antibody sequences can be inserted, for transfection into host cells which are then selected for integration of the desired genes. Lonza offers a CHOK1SV host cell line, although other host cells such as CHOK1 may be used.

Vectors may contain appropriate regulatory sequences, including promoter sequences, terminator sequences, polyadenylation sequences, enhancer sequences, marker genes and other sequences as appropriate. Promoters and other regulatory elements for recombinant gene expression recently reviewed by Gupta et al., Biotechnology Advances 2019. A promoter and/or other element identified in Table 1 of Gupta et al., 2019 may be used in the present invention.

Nucleic acid encoding a polypeptide or antibody can be introduced into a host cell. Nucleic acid can be introduced to eukaryotic cells by various methods, including calcium phosphate transfection, DEAE-Dextran, electroporation, liposome-mediated transfection and transduction using retrovirus or other virus, e.g. vaccinia or, for insect cells, baculovirus. Introducing nucleic acid in the host cell, in particular a eukaryotic cell may use a viral or a plasmid based system. The plasmid system may be maintained episomally or may be incorporated into the host cell or into an artificial chromosome. Incorporation may be either by random or targeted integration of one or more copies at single or multiple loci. For bacterial cells, suitable techniques include calcium chloride transformation, electroporation and transfection using bacteriophage. The introduction may be followed by expressing the nucleic acid, e.g., by culturing host cells under conditions for expression of the gene, then optionally isolating or purifying the antibody.

Nucleic acid of the invention may be integrated into the genome (e.g. chromosome) of the host cell. Integration may be promoted by inclusion of sequences that promote recombination with the genome, in accordance with standard techniques. Nucleic acid encoding a polypeptide or antibody may be integrated into genomic DNA of a host (e.g., CHO) cell, e.g., into chromosomal DNA, and the resulting recombinant cell may be cultured to express the polypeptide or antibody. A cell line development process may comprise introducing nucleic acid encoding the polypeptide or antibody into multiple host cells, and selecting a cell line which expresses a desired level of polypeptide or antibody at the desired yield (e.g., at least 0.5 g/L or at least 1 g/L). Preferably the cell line will retain stable expression over a number of generations in cell culture, and thus it may maintain these levels of production over at least 60 generations for example.

The present invention also provides a method that comprises using nucleic acid described herein in an expression system in order to express the polypeptide or antibody. Desirably, the polypeptide or antibody is expressed at a yield of at least 0.5 g/L in the cell supernatant after initial fermentation, preferably at a yield of >2 g/L. Solubility should be >10 mg/ml, preferably >50 mg/ml, without significant aggregation or degradation of the molecules.

To provide medicines suitable for global treatment, antibodies can be produced on a large scale, for instance in cell culture volumes of at least 100 litres or at least 200 litres, e.g., between 100-250 litres. Batch culture, particularly fed-batch culture, is now commonly used for production of biotherapeutics for clinical and commercial use, and such methods may suitably be used in the present invention to generate the antibodies, followed by purification and formulation steps as noted herein. Bioreactors may be metal (e.g., stainless steel) vessels or may be single-use bioreactors.

### Formulation and Administration

A polypeptide or antibody according to the present invention, and its encoding nucleic acid, will usually be provided in isolated form. VL domains, antibody light chains, antibodies and nucleic acids may be provided purified from their natural environment or their production environment. Isolated polypeptides and isolated nucleic acid will be free or substantially free of material with which they are naturally associated, such as other polypeptides or nucleic acids with which they are found in vivo, or the environment in which they are prepared (e.g., cell culture) when such preparation is by recombinant DNA technology in vitro. Optionally an isolated polypeptide or nucleic acid (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature.

A polypeptide or antibody may be purified (e.g., from cell culture supernatant) by protein A chromatography and/or ion exchange chromatography. Optionally, a bispecific antibody is be produced by a method comprising

expressing two antibody heavy chains and common light chain from cultured host cells comprising encoding nucleic acids,

obtaining cell culture comprising the bispecific antibody and monospecific antibodies assembled from the antibody heavy chains and common light chain,

isolating the bispecific antibody and monospecific antibodies from the cell culture (e.g., using protein A chromatography), and

purifying the bispecific antibody from the monospecific antibodies (e.g., using cation exchange chromatography).

Antibodies or their encoding nucleic acids may be formulated with diluents or adjuvants and still for practical purposes be isolated—for example they may be mixed with carriers if used to coat microtitre plates for use in immunoassays, and may be mixed with pharmaceutically acceptable carriers or diluents when used in therapy. As described elsewhere herein, other active ingredients may also be included in therapeutic preparations. Antibodies may be glycosylated, either naturally in vivo or by systems of heterologous eukaryotic cells such as CHO cells, or they may be (for example if produced by expression in a prokaryotic cell) unglycosylated. The invention encompasses antibodies having a modified glycosylation pattern.

Typically, an isolated product constitutes at least about 5%, at least about 10%, at least about 25%, or at least about 50% of a given sample. An antibody may be substantially free from proteins or polypeptides or other contaminants that are found in its natural or production environment that would interfere with its therapeutic, diagnostic, prophylactic, research or other use.

As discussed elsewhere herein, expression of antibody heavy and light chains for a bispecific antibody may generate unwanted homodimeric species in addition to the active heterodimeric bispecific antibody (e.g., for a bispecific antibody for FIX and FX, homodimers would be anti-FIX and anti-FX antibodies). Preferably a bispecific is provided in a composition in which the heterodimeric bispecific antibody is represents at least 95% of the total antibody, with homodimeric antibody contaminants being present at 5% or less. The composition may comprise at least 98% or at least 99% heterodimeric bispecific, with homodimeric contaminants representing 0-2% or 0-1% respectively.

The invention provides therapeutic compositions comprising the antibodies described herein. Therapeutic compositions comprising nucleic acid encoding such antibodies are also provided. Encoding nucleic acids are described in more detail elsewhere herein and include DNA and RNA, e.g., mRNA. Cells containing nucleic acid encoding the antibody, optionally wherein the nucleic acid is stably integrated into the genome, thus represent medicaments for therapeutic use in a patient. Nucleic acid encoding the antibody may be introduced into human cells derived from the intended patient and modified ex vivo. Administration of cells containing the encoding nucleic acid to the patient provides a reservoir of cells capable of expressing the antibody, which may provide therapeutic benefit over a longer term compared with administration of isolated nucleic acid or the isolated antibody. Nucleic acid encoding the antibody may be provided for use in gene therapy, comprising introducing the encoding nucleic acid into cells of the patient in vivo, so that the nucleic acid is expressed in the patient's cells and provides a therapeutic effect.

Compositions may contain suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTINT™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311. Compositions may comprise the antibody or nucleic acid in combination with medical injection buffer.

Antibodies or their encoding nucleic acids, may be formulated for the desired route of administration to a patient, e.g., in liquid (optionally aqueous solution) for injection.

Various delivery systems are known and can be used to administer the pharmaceutical composition of the invention. Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The antigen-binding molecules are preferably administered by subcutaneous injection.

The pharmaceutical composition can be also delivered in a vesicle, in particular a liposome (see Langer (1990) Science 249:1527-1533; Treat et al. (1989) in Liposomes in the Therapy of Infectious Disease and Cancer, Lopez Berestein and Fidler (eds.), Liss, New York, pp. 353-365; Lopez-Berestein, ibid., pp. 317-327; see generally ibid.).

In certain situations, the pharmaceutical composition can be delivered in a controlled release system. In one embodiment, a pump may be used (see Langer, supra; Sefton (1987) CRC Crit. Ref. Biomed. Eng. 14:201). In another embodiment, polymeric materials can be used; see, Medical Applications of Controlled Release, Langer and Wise (eds.), CRC Pres., Boca Raton, Fla. (1974). In yet another embodiment, a controlled release system can be placed in proximity of the composition's target, thus requiring only a fraction of the systemic dose (see, e.g., Goodson, in Medical Applications of Controlled Release, supra, vol. 2, pp. 115-138, 1984).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule. A pharmaceutical composition of the present invention can be delivered subcutaneously or intravenously with a standard needle and syringe. It is envisaged that treatment will not be restricted to use in the clinic. Therefore, subcutaneous injection using a needle-free device is also advantageous. With respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded. Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Ind.), NOVOPEN™I, II and Ill (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPENT™, OPTIPEN PRO™, OPTIPEN STARLET™, and OPTICLIKT™ (Sanofi-Aventis, Frankfurt, Germany), to name only a few. Examples of disposable pen delivery devices having applications in subcutaneous delivery of a pharmaceutical composition of the present invention include, but certainly are not limited to the SOLOSTAR™ pen (Sanofi-Aventis), the FLEXPEN™ (Novo Nordisk), and the KWIKPEN™ (Eli Lilly).

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, the aforesaid antibody may be contained in about 5 to about 100 mg and in about 10 to about 250 mg for the other dosage forms.

The antibody, nucleic acid, or composition comprising it, may be contained in a medical container such as a phial, syringe, IV container or an injection device. In an example, the antibody, nucleic acid or composition is in vitro, and may be in a sterile container. In an example, a kit is provided comprising the antibody, packaging and instructions for use in a therapeutic method as described herein.

One aspect of the invention is a composition comprising a polypeptide, antibody or nucleic acid of the invention and one or more pharmaceutically acceptable excipients, examples of which are listed above. “Pharmaceutically acceptable” refers to approved or approvable by a regulatory agency of the USA Federal or a state government or listed in the U.S. Pharmacopeia or other generally recognized pharmacopeia for use in animals, including humans. A pharmaceutically acceptable carrier, excipient, or adjuvant can be administered to a patient, together with any antibody or polypeptide molecule described herein, and does not destroy the pharmacological activity thereof and is nontoxic when administered in doses sufficient to deliver a therapeutic amount of the agent.

In some embodiments, the polypeptide or antibody will be the sole active ingredient in a composition according to the present invention. Thus, a composition may consist of the antibody or it may consist of the antibody with one or more pharmaceutically acceptable excipients. However, compositions according to the present invention optionally include one or more additional active ingredients.

### Clauses and Statements of Invention

This invention relates to the expression of lambda antibody light chains with non-native leader sequences, wherein the light chain is sequence-engineered to restore its native N terminus, and to immunoglobulin lambda variable domain sequences comprising an N terminal deletion for expression with non-native N terminal signal peptides. The following numbered clauses and numbered statements present embodiments of the invention and are part of the description.

### Descriptive Clauses

1. A polypeptide comprising an antibody light chain variable (VL) domain with an N-terminal signal peptide, wherein

the VL domain comprises a sequence derived from a v λ gene segment and a sequence derived from a j gene segment, wherein

the N-terminal signal peptide differs from the native N-terminal signal peptide for said A v gene segment, and wherein

the residue corresponding to IMGT position 1 of the VL domain is absent.

2. A polypeptide according to clause 1, wherein the v λ gene segment is a human v λ gene segment.
3. A polypeptide according to clause 1 or clause 2, wherein the j gene segment is a human j λ gene segment.
4. A polypeptide according to any of clauses 1 to 3, wherein the first encoded residue (IMGT position 1) of the v λ gene segment is Ser.
5. A polypeptide according to any of clauses 1 to 3, wherein the v λ gene segment is a vertebrate IGLV3 family gene segment.
6. A polypeptide according to clause 5, wherein the v λ gene segment is IGLV3-21, IGLV3-1, IGLV3-9, IGLV3-10, IGLV3-12, IGLV3-13, IGLV3-16, IGLV3-19, IGLV3-22, IGLV3-25 or IGLV3-27.
7. A polypeptide according to clause 5 or clause 6, wherein the first two encoded residues (IMGT positions 1 to 2) of the v λ gene segment are Ser Tyr.
8. A polypeptide according to clause 7, wherein the first three encoded residues (IMGT positions 1 to 3) of the v λ gene segment are Ser Tyr Val.
9. A polypeptide according to clause 6 or clause 7, wherein the v λ gene segment is human IGLV3-21.
10. A polypeptide according to any preceding clause, wherein the signal peptide has a length of 20 amino acids.
11. A polypeptide according to any preceding clause, wherein the signal peptide is not a human λ v gene segment signal peptide.
12. A polypeptide according to clause 11, wherein the signal peptide is not a λ v gene segment signal peptide.
13. A polypeptide according to any preceding clause, wherein the signal peptide comprises a C-terminal Cys residue.
14. A polypeptide according to clause 13, wherein the signal peptide comprises a C-terminal sequence Ala Arg Cys.
15. A polypeptide according to clause 14, wherein the signal peptide comprises a C-terminal sequence Thr Asp Ala Arg Cys.
16. A polypeptide according to any preceding clause, wherein the signal peptide is a κ v gene segment signal peptide.
17. A polypeptide according to clause 16, wherein the signal peptide is a mouse κ v gene segment signal peptide.
18. A polypeptide according to clause 17, wherein the signal peptide is

SEQ ID NO: 62
MSVPTQVLGLLLLWLTDARC.

19. A polypeptide according to any preceding clause, wherein the polypeptide is an antibody light chain comprising the VL domain and a light chain constant (CL) domain.
20. A polypeptide according to clause 19, wherein the CL domain is a λCL domain.
21. A polypeptide according to any preceding clause, wherein the VL domain is the N0325L VL domain.
22. A polypeptide according to clause 21, consisting of amino acid sequence

SEQ ID NO: 66
MSVPTQVLGLLLLWLTDARCYVLTQPPSVSVAPGETARITCGGDNIGRK
SVYWYQQKSGQAPVLVIYYDSDRPSGIPERFSGSNSGNTATLTISRVEA
GDEADYYCQVWDGSSDHWWFGGGTKLTVL

or comprising said sequence as an N-terminal domain.

23. A polypeptide according to clause 22, which is an antibody λ light chain consisting of or comprising amino acid sequence

SEQ ID NO: 67
MSVPTQVLGLLLLWLTDARCYVLTQPPSVSVAPGETARITCGGDNIGRK
SVYWYQQKSGQAPVLVIYYDSDRPSGIPERFSGSNSGNTATLTISRVEA
GDEADYYCQVWDGSSDHVWFGGGTKLTVLGQPKAAPSVTLFPPSSEELQ
ANKATLVCLISDFYPGAVTVAWKADSSPVKAGVETTTPSKQSNNKYAAS
SYLSLTPEQWKSHKSYSCQVTHEGSTVEKTVAPTECS.

24. Nucleic acid encoding a polypeptide according to any preceding clause.
25. Nucleic acid according to clause 24, wherein the nucleic acid is cDNA.
26. Nucleic acid according to clause 24, wherein the nucleic acid is genomic DNA comprising introns.
27. Nucleic acid according to any of clauses 24 to 26, comprising nucleotide sequence ATGTCTGTGCCTACACAGGTTCTGGGACTGCTGCTGCTGTGGCTGACCGACGCCAGATGT SEQ ID NO: 64 encoding the signal peptide.
28. Nucleic acid according to any of clauses 24 to 27, comprising nucleotide sequence TACGTGCTGACCCAGCCTCCTTCCGTGTCTGTTGCTCCTGGCGAGACAGCCAGAATCACC TGTGGCGGCGATAACATCGGCCGGAAGTCCGTGTACTGGTATCAGCAGAAGTCCGGCCA GGCTCCTGTGCTGGTCATCTACTACGACTCCGACCGGCCTTCTGGCATCCCTGAGAGATT CTCCGGCTCCAACTCCGGCAATACCGCCACACTGACCATCTCCAGAGTGGAAGCTGGCGA CGAGGCCGACTACTACTGCCAAGTGTGGGACGGCTCCTCTGACCACTGGGTTTTCGGCGG AGGCACCAAGCTGACAGTGCTG SEQ ID NO: 100 encoding the VL domain.
29. Nucleic acid according to clause 28, comprising nucleotide sequence

SEQ ID NO: 65
ATGTCTGTGCCTACACAGGTTCTGGGACTGCTGCTGCTGTGGCTGACCGA
CGCCAGATGTTACGTGCTGACCCAGCCTCCTTCCGTGTCTGTTGCTCCTG
GCGAGACAGCCAGAATCACCTGTGGCGGCGATAACATCGGCCGGAAGTCC
GTGTACTGGTATCAGCAGAAGTCCGGCCAGGCTCCTGTGCTGGTCATCTA
CTACGACTCCGACCGGCCTTCTGGCATCCCTGAGAGATTCTCCGGCTCCA
ACTCCGGCAATACCGCCACACTGACCATCTCCAGAGTGGAAGCTGGCGAC
GAGGCCGACTACTACTGCCAAGTGTGGGACGGCTCCTCTGACCACTGGGT
TTTCGGCGGAGGCACCAAGCTGACAGTGCTG.

30. A host cell in vitro comprising nucleic acid according to any of clauses 24 to 29.
31. A cell according to clause 30 which is a mammalian cell.
32. A cell according to clause 31 which is a CHO cell.
33. A cell according to clause 31 which is a human cell.
34. A cell according to clause 33 which is a HEK cell.
35. A cell according to any of clauses 30 to 34, wherein said nucleic acid is integrated into chromosomal DNA of the cell.
36. An in vitro population of cells according to any of clauses 30 to 35.
37. A population of cells according to clause 36, wherein

the cells express the encoded polypeptide, and wherein

on expression in the cells, the signal peptide is cleaved from the polypeptide to provide a mature VL domain comprising an N-terminal residue corresponding to IMGT position 2.

38. A method of expressing a polypeptide comprising a VL domain, the method comprising

culturing a population of cells according to clause 36 or clause 37 under conditions for expression of the polypeptide, wherein

the N-terminal signal peptide is cleaved off the VL domain to provide a polypeptide comprising a mature VL domain, wherein

the N-terminal residue of the polypeptide comprising the mature VL domain is IMGT position 2 of the VL domain.

39. A method of expressing an antibody comprising a VH domain and a VL domain, wherein the VL domain comprises a sequence derived from a v λ gene segment and a sequence derived from a j gene segment, the method comprising

providing a population of cells according to clause 36 or clause 37, wherein the cells further comprise nucleic acid encoding the VH domain,

culturing said population of cells under conditions for expression of the polypeptide comprising the VL domain and for expression of the VH domain, wherein

the N-terminal signal peptide is cleaved off the VL domain to provide a polypeptide comprising a mature VL domain, wherein

the N-terminal residue of the polypeptide comprising the mature VL domain is IMGT position 2 of the VL domain, and wherein

the VL domain assembles with the VH domain to provide said antibody.

40. A method according to clause 38 or clause 39, wherein the polypeptide or antibody is secreted from the cells.
41. A method according to any of clauses 38 to 40, comprising isolating the polypeptide or antibody from the population of cells.
42. A method according to clause 41, comprising purifying the polypeptide or antibody by one or more steps of protein chromatography.
43. A method according to any of clauses 38 to 42, wherein the antibody is an IgG.
44. A method according to any of clauses 38 to 43, wherein the antibody is a multispecific antibody.
45. A method of expressing a bispecific antibody comprising two heavy chains each comprising a different VH domain, and a common light chain comprising a VL domain, wherein the VL domain comprises a sequence derived from a v λ gene segment and a sequence derived from a j gene segment, the method comprising

providing a population of cells according to clause 36 or clause 37, wherein the cells further comprise nucleic acid encoding the two heavy chains,

culturing said population of cells under conditions for expression of the antibody heavy and light chains, wherein

the N-terminal signal peptide is cleaved off the VL domain to provide an antibody light chain comprising a mature VL domain, wherein

the N-terminal residue of the polypeptide comprising the mature VL domain is IMGT position 2 of the VL domain, and wherein

the light chain domain assembles with each of the two heavy chains to provide said bispecific antibody.

46. A method according to clause 45, wherein the two heavy chains comprise VH domains N1280H and T0999H respectively and wherein the light chain comprises VL domain 0325L.
47. A method according to clause 46, wherein the antibody is an IgG4 comprising the N1280H heavy chain, the T0999H heavy chain and the 0325L light chain.
48. A method according to clause 45, wherein the two heavy chains comprise VH domains N1441H and T0999H respectively and wherein the light chain comprises VL domain 0325L.
49. A method according to clause 46, wherein the antibody is an IgG4 comprising the N1441H heavy chain, the T0999H heavy chain and the 0325L light chain.
50. An antibody VL domain obtained by cleavage of the N-terminal signal peptide from the polypeptide of any of clauses 1 to 23.
51. An isolated antibody comprising a VL domain according to clause 50.
52. An isolated antibody obtained by the method of any of clauses 39 to 49.
53. An isolated antibody comprising a VH domain and a VL domain, wherein the VL domain comprises a sequence derived from a v λ gene segment and a sequence derived from a j gene segment, and wherein the N-terminal residue of the VL domain is the residue corresponding to IMGT position 2.
54. An antibody according to clause 53, wherein the v λ gene segment is a human Vλ3-21 gene segment.
55. An antibody according to clause 54, wherein the VL domain amino acid sequence is the 0325L VL domain amino acid sequence.
56. An antibody according to clause 55, wherein the antibody comprises a λ light chain wherein the amino acid sequence of said light chain is the 0325L light chain amino acid sequence.
57. An antibody according to any of clauses 53 to 56, which is a bispecific antibody comprising two heavy chains each comprising a different VH domain, and a common light chain comprising said VL domain.
58. An antibody according to clause 57, wherein the bispecific antibody comprises

a heavy chain comprising the N1280H VH domain;

a heavy chain comprising the T0999H VH domain; and

a common light chain comprising the 0325L VL domain.

59. An antibody according to clause 58, comprising the N1280H heavy chain, the T0999H heavy chain and the 0325L common light chain.
60. An antibody according to clause 57, wherein bispecific antibody comprises

a heavy chain comprising the N1441H VH domain;

a heavy chain comprising the T0999H VH domain; and

a common light chain comprising the 0325L VL domain.

61. An antibody according to clause 60, comprising the N1441H heavy chain, the T0999H heavy chain and the 0325L common light chain.
62. A method of formulating a pharmaceutical composition, comprising combining an antibody with a pharmaceutically acceptable excipient, wherein the antibody is an antibody according to any of clauses 51 to 61.
63. A method of producing, or of improving stability and/or antigen-binding of, an antibody comprising a VH domain and a VL domain, wherein the VL domain comprises a sequence derived from a v λ gene segment and a sequence derived from a j gene segment, the method comprising

providing nucleic acid encoding said antibody, comprising a nucleotide sequence encoding said VL domain with an N-terminal signal peptide, wherein the codon for the residue corresponding to IMGT position 1 of said VL domain is deleted, N-terminal signal peptide is not the native N-terminal signal peptide for said λ v gene segment, and

expressing said nucleic acid to provide an antibody comprising the VH domain and VL domain, wherein the N-terminal residue of the VL domain is the residue corresponding to IMGT position 2, and optionally isolating and purifying said antibody.

### Descriptive Statements

1. A polypeptide comprising an antibody light chain variable (VL) domain with an N-terminal signal peptide, wherein

the VL domain comprises a sequence derived from a v λ gene segment and a sequence derived from a j gene segment, wherein

the N-terminal signal peptide differs from the native N-terminal signal peptide for said A v gene segment, and wherein

the VL domain comprises an N-terminal truncation relative to the amino acid sequence encoded by the v λ gene segment starting at IMGT position 1.

2. A polypeptide according to statement 1, wherein the residue corresponding to IMGT position 1 of the VL domain is absent and the N-terminal residue of the VL domain corresponds to IMGT position 2.
3. A polypeptide according to statement 1 or statement 2, wherein the v λ gene segment is a human v λ gene segment.
4. A polypeptide according to any preceding statement, wherein the j gene segment is a human j λ gene segment.
5. A polypeptide according to any of statements 1 to 4, wherein the first encoded residue (IMGT position 1) of the v λ gene segment is Ser, optionally wherein the v λ gene segment is a vertebrate IGLV3 family gene segment.
6. A polypeptide according to statement 5, wherein the v λ gene segment is IGLV3-21, IGLV3-1, IGLV3-9, IGLV3-10, IGLV3-12, IGLV3-13, IGLV3-16, IGLV3-19, IGLV3-22, IGLV3-25 or IGLV3-27.
7. A polypeptide according to statement 5 or statement 6, wherein the first two encoded residues (IMGT positions 1 to 2) of the v λ gene segment are Ser Tyr.
8. A polypeptide according to statement 7, wherein the first three encoded residues (IMGT positions 1 to 3) of the v λ gene segment are Ser Tyr Val.
9. A polypeptide according to statement 6 or statement 7, wherein the v λ gene segment is human IGLV3-21.
10. A polypeptide according to any preceding statement, wherein the signal peptide has a length of 20 amino acids.
11. A polypeptide according to any preceding statement, wherein the signal peptide is not a human λ v gene segment signal peptide.
12. A polypeptide according to statement 11, wherein the signal peptide is not a λ v gene segment signal peptide.
13. A polypeptide according to any preceding statement, wherein the signal peptide comprises a C-terminal Cys residue.
14. A polypeptide according to statement 13, wherein the signal peptide comprises a C-terminal sequence Ala Arg Cys.
15. A polypeptide according to statement 14, wherein the signal peptide comprises a C-terminal sequence Thr Asp Ala Arg Cys.
16. A polypeptide according to any preceding statement, wherein the signal peptide is a κ V gene segment signal peptide.
17. A polypeptide according to statement 16, wherein the signal peptide is a mouse κ V gene segment signal peptide.
18. A polypeptide according to statement 17, wherein the signal peptide is

SEQ ID NO: 62
MSVPTQVLGLLLLWLTDARC

19. A polypeptide according to any of statements 1 to 4, wherein the signal peptide is SEQ ID NO: 74.
20. A polypeptide according to any preceding statement, wherein the polypeptide is an antibody light chain comprising the VL domain and a light chain constant (CL) domain, optionally wherein the CL domain is a λCL domain.
21. A polypeptide according to any preceding statement, wherein the VL domain is the N0325L VL domain SEQ ID NO: 101.
22. A polypeptide according to statement 21, consisting of amino acid sequence SEQ ID NO: 66 or comprising said sequence as an N-terminal domain.
23. A polypeptide according to statement 22, which is an antibody λ light chain consisting of or comprising amino acid sequence SEQ ID NO: 67.
24. A polypeptide according to any of statements 1 to 20, wherein the VL domain is the LARI VL domain SEQ ID NO: 117.
25. Nucleic acid encoding a polypeptide according to any preceding statement.
26. Nucleic acid according to statement 25, wherein the nucleic acid is cDNA.
27. Nucleic acid according to statement 25, wherein the nucleic acid is genomic DNA comprising introns.
28. Nucleic acid according to any of statements 25 to 27, comprising nucleotide sequence SEQ ID NO: 64 encoding the signal peptide.
29. Nucleic acid according to any of statements 25 to 28, comprising nucleotide sequence SEQ ID NO: 100 encoding the VL domain.
30. Nucleic acid according to statement 29, comprising nucleotide sequence SEQ ID NO: 65.
31. A host cell in vitro comprising nucleic acid according to any of statements 25 to 30.
32. A cell according to statement 31 which is a mammalian cell.
33. A cell according to statement 32 which is a CHO cell.
34. A cell according to statement 31 which is a human cell, optionally a HEK cell.
35. A cell according to any of statements 31 to 34, wherein said nucleic acid is integrated into chromosomal DNA of the cell.
36. An in vitro population of cells according to any of statements 31 to 35.
37. A population of cells according to statement 36, wherein

the cells express the encoded polypeptide, and wherein

on expression in the cells, the signal peptide is cleaved from the polypeptide to provide a mature VL domain comprising an N-terminal residue corresponding to the N-terminal residue of a VL domain encoded by the same v λ gene segment when expressed with and cleaved from its native signal peptide, optionally wherein the N-terminal residue is IMGT position 2 or IMGT position 3.

38. A method of expressing a polypeptide comprising a VL domain, the method comprising

culturing a population of cells according to statement 36 or statement 37 under conditions for expression of the polypeptide, wherein

the N-terminal signal peptide is cleaved off the VL domain to provide a polypeptide comprising a mature VL domain, wherein

the N-terminal residue of the polypeptide comprising the mature VL domain is corresponds to the N-terminal residue of a VL domain encoded by the same v λ gene segment when expressed with and cleaved from its native signal peptide, optionally wherein the N-terminal residue is IMGT position 2 or IMGT position 3 of the VL domain.

39. A method of expressing an antibody comprising a VH domain and a VL domain, wherein the VL domain comprises a sequence derived from a v λ gene segment and a sequence derived from a j gene segment, the method comprising

providing a population of cells according to statement 36 or statement 37, wherein the cells further comprise nucleic acid encoding the VH domain,

culturing said population of cells under conditions for expression of the polypeptide comprising the VL domain and for expression of the VH domain, wherein

the N-terminal signal peptide is cleaved off the VL domain to provide a polypeptide comprising a mature VL domain, wherein

the N-terminal residue of the polypeptide comprising the mature VL domain corresponds to the N-terminal residue of a VL domain encoded by the same v λ gene segment when expressed with and cleaved from its native signal peptide, optionally wherein the N-terminal residue is IMGT position 2 or IMGT position 3 of the VL domain, and wherein

the VL domain assembles with the VH domain to provide said antibody.

40. A method according to statement 38 or statement 39, wherein the polypeptide or antibody is secreted from the cells.
41. A method according to any of statements 38 to 40, comprising isolating the polypeptide or antibody from the population of cells.
42. A method according to statement 41, comprising purifying the polypeptide or antibody by one or more steps of protein chromatography.
43. A method according to any of statements 38 to 42, wherein the antibody is an IgG.
44. A method according to any of statements 38 to 43, wherein the antibody is a multispecific antibody.
45. A method of expressing a bispecific antibody comprising two heavy chains each comprising a different VH domain, and a common light chain comprising a VL domain, wherein the VL domain comprises a sequence derived from a v λ gene segment and a sequence derived from a j gene segment, the method comprising

providing a population of cells according to statement 36 or statement 37, wherein the cells further comprise nucleic acid encoding the two heavy chains,

culturing said population of cells under conditions for expression of the antibody heavy and light chains, wherein

the N-terminal signal peptide is cleaved off the VL domain to provide an antibody light chain comprising a mature VL domain, wherein

the N-terminal residue of the polypeptide comprising the mature VL domain corresponds to the N-terminal residue of a VL domain encoded by the same v λ gene segment when expressed with and cleaved from its native signal peptide, optionally wherein the N-terminal residue is IMGT position 2 or IMGT position 3 of the VL domain, and wherein

the light chain domain assembles with each of the two heavy chains to provide said bispecific antibody.

46. A method according to statement 45, wherein the N-terminal residue of the polypeptide comprising the mature VL domain is IMGT position 2 of the VL domain.
47. A method according to statement 46, wherein the two heavy chains comprise VH domains N1441H SEQ ID NO: 83 and T0999H SEQ ID NO: 87 respectively and wherein the light chain comprises VL domain 0325L SEQ ID NO: 101.
48. A method according to statement 47, wherein the antibody is an IgG4 comprising the N1441H heavy chain SEQ ID NO: 85, the T0999H heavy chain SEQ ID NO: 89 and the 0325L light chain SEQ ID NO: 103.
49. A method according to any of statements 45 to 48, wherein the signal peptide is SEQ ID NO: 62.
50. An antibody VL domain obtained by cleavage of the N-terminal signal peptide from the polypeptide of any of statements 1 to 24.
51. An isolated antibody comprising a VL domain according to statement 50.
52. An isolated antibody obtained by the method of any of statements 39 to 49.
53. A method of improving stability and/or antigen-binding, or of reducing immunogenicity, of an antibody comprising a VH domain and a VL domain, wherein the VL domain comprises a sequence derived from a v λ gene segment and a sequence derived from a j gene segment, the method comprising

providing nucleic acid encoding said antibody, comprising a nucleotide sequence encoding said VL domain with an N-terminal signal peptide, wherein the codon or codons for the residue or residues corresponding to IMGT position 1 and optionally IMGT position 2 of said VL domain is or are deleted, and wherein the N-terminal signal peptide is not the native N-terminal signal peptide for said λ v gene segment, and

expressing said nucleic acid to provide an antibody comprising the VH domain and VL domain, wherein the N-terminal residue of the VL domain is the residue corresponding to IMGT position 2 or IMGT position 3, and optionally isolating and purifying said antibody.

54. A method comprising
(i) providing a nucleotide sequence encoding an antibody light chain variable (VL) domain,
(ii) providing a first DNA molecule comprising the said nucleotide sequence preceded by a leader sequence encoding the native signal peptide,
(iii) providing a second DNA molecule comprising the said nucleotide sequence preceded by a leader sequence encoding a non-native signal peptide,
(iv) expressing the first and second DNA in host cells to produce first and second polypeptides respectively comprising the VL domain,
(v) comparing VL domain amino acid sequences of the first and second polypeptides expressed from the host cells,
(vi) identifying an N-terminal truncation in the sequence of the first polypeptide compared with the second polypeptide,
(vii) providing an engineered nucleotide sequence encoding the sequence of the first polypeptide, including the N-terminal truncation,
(viii) providing a third DNA molecule comprising the said engineered nucleotide sequence preceded by a leader sequence encoding a non-native signal peptide, and
(ix) expressing the third DNA molecule in a host cell to produce a third polypeptide comprising the encoded VL domain, wherein the amino acid sequence of the third polypeptide is identical to that of the first polypeptide.
55. A method according to statement 54, comprising
(x) isolating and purifying the third polypeptide from the host cell.
56. A method according to statement 54 or statement 55, comprising expressing an antibody comprising a VH domain and the said VL domain.
57. A method according to statement 56, wherein an IgG antibody is produced by co-expressing an antibody heavy chain comprising the VH domain and an antibody light chain comprising the VL domain in the host cells.

### EXAMPLES

### Example 1 Creation and Characterisation of λ Antibodies Including 0128L VL Domain

Bispecific antibodies recognising coagulation factors IX and X (FIX and FX) were generated in IgG format. One arm of the IgG comprises a VH-VL domain pair specific for FIX and the other arm of the IgG comprises a VH-VL domain pair specific for FX. The bispecific antibodies comprise two different heavy chains, each with a different VH domain, and a common light chain comprising the 0128L VL domain which pairs with each of the two different VH domains. The 0128L VL domain was generated by immunisation of mice transgenic for human immunoglobulin heavy and λ light chain gene segments. Comparison of the nucleotide sequence encoding the 0128L VL domain with germline gene segments indicates that it was derived from recombination of human λ gene segments IGLV3-21*d01 and IGLJ2*01. Generation of the 0128L light chain was described in detail in WO2018/234575.

Bispecific antibodies comprising a common λ light chain including the 0128L VL domain were screened for FVIIIa-mimetic activity, i.e., ability to enhance (catalyse) the FIXa-mediated activation of FX to FXa in vitro by enzymatic “FXase” assay. In this assay, the test bispecific molecule is contacted with FIXa and FX in the presence of phospholipid, under conditions suitable for formation of FXa. A substrate for FXa is added which, when cleaved by FXa, generates a detectable product. Detection of this product in the presence of test bispecific antibody is compared with a negative control in which no test antibody is present (a control antibody may be included). The detected signal is quantified by recording absorbance of the reaction solution at 405 nm. Absorbance is measured across a range of antibody concentrations in the assay and an EC50 value is calculated as a measure of the bispecific antibody potency in this assay. Significant difference of EC50 between test antibody and control indicates that the test antibody is able to enhance FIXa-mediated activation of FX.

Antibodies tested in this assay were initially generated from expression of nucleic acid encoding the heavy and light chains with their native signal peptides. Thus, the 0128L light chain was expressed from nucleic acid encoding the 0128L VL domain with its native vλ3-21 leader. Antibodies were expressed in HEK cells.

Bispecific antibodies which showed strong activity in FXase assay were selected.

The nomenclature used herein for the anti-FIXxFX bispecific antibodies which have a common light chain is IXAX-nnnn.tttt.llll, wherein nnnn is a 4 digit numerical identifier of the anti-FIX VH domain, tttt is a 4 digit identifier of the anti-FX VH domain, and III is a 4 digit numerical identifier of the common VL domain. Sequences of VH and VL domains and heavy and light chains of IXAX antibodies are shown in appended Table S and elsewhere herein.

### Example 2 Expression of λ Antibody with Different Leader Sequences and 0128L VL Domain

For industrial manufacturing it is common to transfer the coding sequence for the mature antibody polypeptides to a transfection vector comprising a leader sequence. The VL domain sequence is thus expressed with a non-native leader.

When we switched the native leader sequences of our bispecific antibodies to alternative leader sequences for manufacturing purposes, we observed a decrease in functional activity in the FXase assay. This was not the result of a change in antibody expression, which remained constant (see Example 7). Unexpectedly, we saw a drop in FVIII mimetic activity when a kappa light chain leader was used to express the 0128L common light chain.

In more detail, a panel of 21 bispecific antibodies comprising a range of different anti-FX heavy chains, each in combination with the N0128H anti-FIX heavy chain and 0128L common light chain were screened in the FXase assay. Two different 0128L common light chain constructs were used to express the N0128L common light chain, one with the native leader, and the other with a mouse kappa light chain leader. Sequences are shown in appended Table P.

The bispecific antibodies were expressed in HEK cells, using vectors encoding the anti-FIX and anti-FX heavy chains and the common light chain N0128_IgL with the selected light chain leader.

Bispecific antibodies were expressed using a high-throughput 2 ml deep-well block transfection method. Following expression and harvest, bispecific antibodies were purified from HEK media using Protein A chromatography, protein quantified by OD280 and all samples normalised for protein concentration.

A significant drop of FXa generation activities was observed in bispecific antibodies in which the mouse kappa light chain leader was used to express the common light chain, compared with those which the native leader was used to express the common light chain. FIG. 3.

### Example 3 Identification of Leader-Dependent N-Terminal Clipping in 0128L

The structural integrity of therapeutic monoclonal antibodies can be compromised by multiple types of post-translational modifications which result in product heterogeneity. Mass spectrometry (MS) was used to characterise and evaluate the molecular mass of the bispecific antibodies after cation exchange purification.

Unexpectedly, mass spectrometry analysis of common light chain comprising 0128L which had been expressed with native human Igλ leader sequence identified a specific clipping the N-terminal Ser occurring on the fully assembled bispecific antibody when compared to IMGT reference sequence. Notably, however, the non-native leader sequence retained the N terminal serine when analysed by mass spectrometry.

Bispecific antibody IXAX-1172.0201.0128 was expressed from nucleic acids encoding an anti-FIX heavy chain comprising the N1172H VH domain, an anti-FX heavy chain comprising the T0201H VH domain and a common light chain comprising the 0128L VL domain.

The antibody was expressed using either native leader or mouse kappa light chain leader for the common light chain, and the anti-FIX/anti-FX heterodimer was purified by cation exchange and analysed by mass spectrometry.

When the native leader was used to express the light chain, molecular weights (MW) of anti-FIX/anti-FX heterodimer determined by MS was smaller than the theoretical MW predicted by amino acid sequences of the IXAX-1172.0201.0128 anti-FIX/anti-FX heterodimer. MS results of the common light chain suggested that the N-terminal serine of the common light chain was truncated when native leader is used.

When mouse kappa light chain leader was used to express the light chain, MW of anti-FIX/anti-FX heterodimer determined by MS matched the theoretical MW predicted by amino acid sequences of the anti-FIX/anti-FX IXAX-1172.0201.0128 heterodimer. MS results of the common light chain suggested that the N-terminal serine of the common light chain was intact when the mouse kappa light chain leader is used.

These observations were consistent regardless of whether the bispecific antibody was expressed in either HEK or CHO cells.

Notably, we observed with the full length 0128L (including the N-terminal serine) that cleavage was either complete or not at all as determined by mass spectrometry, depending on which leader sequence was used. This contrasts with an earlier report of leader misprocessing (Gibson et al., 2017), in which a variable cleavage site was reported to account for low levels (4-6%) of truncated light chain in the recovered antibody product.

### Example 4 Mutation Analysis at IMGT Position 1 of 0128L

We hypothesised that removal of the N-terminal serine in the presence of the new leader sequence would restore functional activity. We further investigated the effect of substituting different amino acid residues for the serine at IMGT position 1.

The heavy chains of the anti-FIX binding arm and the anti-FX binding arm of the bispecific antibody were “fixed” as the N1280H VH domain and the T0736H VH domain respectively, while further refinements were made to the common light chain VL domain to identify mutants that would retain the FXa generation activities of the bispecific antibodies when the mouse kappa light chain leader was used.

Table E below identifies mutants of the 0128L VL domain in which one or more residues of the CDRs are mutated to other amino acids. The table shows the name given to each variant VL domain having the identified mutation at IMGT position 1. In each case, residues other than IMGT position 1 were left unchanged. For example, the 0310L VL domain is a Ser1Ala mutant of the 0128L VL domain, i.e., in which the serine (S) at IMGT position 1 in Framework 1 is replaced by alanine (A). The 0325L VL domain is the 0128L VL domain with a deletion at IMGT position 1, i.e., it represents the 0128L VL domain with the N-terminal serine clipped off.

| VL domain variant of N0128L | IMGT residue 1 |
|---|---|
| N0310L | A |
| N0311L | C |
| N0312L | D |
| N0313L | E |
| N0314L | F |
| N0315L | G |
| N0316L | H |
| N0317L | I |
| N0318L | K |
| N0319L | L |
| N0320L | M |
| N0321L | N |
| N0322L | P |
| N0323L | Q |
| N0324L | R |
| N0325L | del |
| N0326L | T |
| N0327L | V |
| N0328L | W |
| N0329L | Y |
| N0128L | S |

Bispecific antibodies, purified by protein A chromatography, were tested for functional activity to look for maintenance of FXa generation activities of the parent bispecific comprising 0128L VL domain when the native leader is used. Newly generated bispecific antibodies were characterised in the FXase assay described in Example 1.

Mutagenesis of light chain framework 1 produced improvements in FVIII mimetic activity. N0325L, which compromises a deletion of the N-terminal serine, was the only mutant that maintained the FXa generation activities of the bispecific antibodies when the mouse kappa light chain leader was used. Bispecific comprising 0325L VL domain demonstrated activity comparable to that of the parent bispecific comprising 0128L VL domain when the native leader was used. FIG. 4.

Thus, in the presence of the alternative leader sequence, deletion of the N-terminal serine demonstrated greater biological activity compared to the same bispecific antibody with the N-terminal serine. This confirms the effect of serine N-terminal deletion on the functionality of the molecule in the presence of the alternative leader sequence.

### Example 5 Expression of Antibody with 0325L VL Domain

Following identification of N0325L, a larger scale transient HEK Expi293 transfection was subsequently carried out at 30 ml, and IXAX bispecific antibodies produced in this HEK expression system confirmed the observations from the small-scale transfection (Example 2).

We also confirmed that IXAX bispecific antibodies with common light chains lacking the N-terminal serine expressed with a non-native leader demonstrated restored functional activity when expressed stably as CHO mini-pools.

Both HEK and CHO cell lines were used to express IXAX antibodies including the 0325L light chain with either native vλ3-21 leader or the non-native mouse κ leader. A codon-optimised leader nucleotide sequence was used (optimised for CHO, and the same sequence used in HEK).

### Expression in HEK Cells

Briefly, 7.5×10⁷ cells (final cell density of 2.5×10⁶ cells/mL) were used for each 30 ml transfection and were incubated in a 37° C. incubator with a humidified atmosphere of 8% CO₂ in air on an orbital shaker rotating at 125-140 rpm for 1 hour. For each transfection sample, lipid-DNA complexes were prepared by mixing 30 μg of plasmid DNA in Opti-MEM® I medium to a total volume of 1.5 mL, followed by gentle mixing. Subsequently, 80 μL of ExpiFectamine™ 293 Reagent was diluted in Opti-MEM® I medium to a total volume of 1.5 mL, followed by gentle mixing. This mixture was incubated for 5 minutes at room temperature. After the 5 minute incubation, the DNA was added to the diluted ExpiFectamine™ 293 Reagent to obtain a total volume of 3 mL. This mixture was mixed gently and incubated for 20-30 minutes at room temperature to allow DNA-ExpiFectamine™ 293 Reagent complexes to form. Once the incubation has completed the DNA-ExpiFectamine™ 293 Reagent complex is added to each shake flask containing Expi293 cells. Cells are incubated in a 37° C. incubator with a humidified atmosphere of 8% CO₂ in air on an orbital shaker rotating at 125-140 rpm. Approximately 16-18 hours post-transfection, 150 μL of ExpiFectamine™ 293 Transfection Enhancer 1 and 1.5 mL of ExpiFectamine™ 293 Transfection Enhancer 2 is added to each flask. Supernatants were harvested 5 to 6 days post-transfection. Following expression and harvest, bispecific antibodies were purified from HEK media using Protein A chromatography, protein quantified by OD280 and samples normalised for protein concentration.

We confirmed IXAX bispecific antibodies with common light chains lacking the N-terminal serine expressed with a non-native leader demonstrated similar levels of functional activity to full-length common light chain expressed with a native leader in HEK cells regardless whether the expression scale was small (2 ml) or larger scale (30 ml).

### Expression in CHO Cells

We utilised the Lonza GS Xceed™ Gene Expression System which uses a single selectable marker—glutamine synthetase (GS), encoded in the vector. CHO-K1 GS-knockout cells were used for transfection. Surviving cells will have integrated the vector in a transcriptionally active locus of the CHO genome—transcribing GS, to synthesise glutamine (essential for survival). The media used for selection is deficient in glutamine but supplemented by methionine sulfoximine (MSX) for stringent selection. MSX potently and irreversibly inhibits the GS enzyme.

Vectors encoding the heavy chains and 0325L common light chain of IXAX antibodies were linearised before transfection into CHO cells and subsequently purified using Phase Lock Gel tubes (Quantabio). The CHOK1SV GS-KO host cells were cultivated in CD-CHO media (Gibco), supplemented with 200 mM L-glutamine (Gibco)—to give a final concentration of 6 mM glutamine. The cells were incubated at 36.5° C., 140 rpm, 7% CO2, 70% humidity. Cell density and viability were measured using the Vi-CELL XR cell viability analyser (Beckman Coulter). Five days before transfection, the cells were sub-cultured at 0.2×106 cells/mL in 200 mL TV CD-CHO 6 mM L-glutamine (Gln). Two days before the transfection, the cells were split into two 400 mL TV at 0.3×106 cells/mL in CD-CHO 6 mM Gln. The day of the transfection, the flasks were measured at 2.5/2.8×106 cells/mL. CHOK1SV GS-KO host cells were transfected with the linearised DNA using Amaxa 4D Nucleofector (Lonza) electroporation following the manufacturers protocol.

Once transfected, cells were incubated at for 24 hours at 36.5° C., 140 rpm, 7% CO2, 70% humidity. On day one post-transfection the pools were centrifuged at 200×g for 5 minutes. The supernatant was removed. The pellet was re-suspended in 30 mL of CD-CHO/SP4/MSX and transferred to new flasks. MSX at a concentration of 75 μM was used for positive selection of CHO cells containing the vectors, thus theoretically expressing the bispecific antibody. The cells were under selection for a total of 29 days. The minipools were incubated in a static incubator (36.5° C., 7% CO2, 70% humidity) for a total of 19 days before the first passage. All pools were kept under selection until the fed-batch overgrow was set-up to promote expression of the bispecific antibody.

For fed batch overgrow (FOG), cells were seeded at 0.2×106 viable cells/mL in 30 mL of pre-warmed CM79 medium in a new culture vessel (manufacturers protocol was followed). The flasks were placed in an incubator (36.5° C., 140 rpm, 7% CO2, 70% humidity). MSX selection at this stage was not included. From day three onwards, the glucose concentration and viable cell concentration were measured daily. Upon reaching >1.5×106 cells/mL, SP76 was added to the cells daily—the volume of SP76 added was determined by the viable cell concentration. 400 g/L D-glucose feed was added daily from day three onwards to prevent glucose depletion. The volume of 400 g/L D-glucose feed added was dependent on the measured glucose concentration. SF54, SF71 and SF77 were added at fixed volumes at regular intervals following the manufacturers recommendation. After 12 days, the 30 mL cultures were harvested. Following expression and harvest, bispecific antibodies were purified from CHO media using Protein A chromatography, protein quantified by OD280 and samples normalised for protein concentration.

### Example 6 Confirmation of Expressed 0325L VL Domain Sequence by Mass Spectrometry

Anti-FIX/anti-FX heterodimeric bispecific antibody generated from CHO cells (see Example 5) was analysed by MS.

When the mouse kappa light chain leader was used to express the 0325L common light chain, MW of the anti-FIX/anti-FX heterodimer determined by MS matched the theoretical MW predicted by amino acid sequences of the anti-FIX/anti-FX heterodimer.

### Example 7 Comparison of Yield for λ Antibodies with and without N-Terminal VL Residue

As detailed in Example 5, vectors comprising DNA encoding bispecific antibody IXAX-1172.0201.0128 were transfected into CHO cells. Two different leader sequences for the 0128L VL domain were used—either a codon-optimised native λ leader or a codon-optimised mouse kappa leader. Cells were selected for stable transfection using 75 μM MSX (methionine sulphoximine) which inhibits the GS protein.

Following stable expression of the bispecific antibodies, minipools were cultured by fed-batch overgrowth to allow bispecific antibody expression at 35 ml scale in shake flask. Following expression and harvest, bispecific antibody was purified from CHO supernatant using Protein A affinity chromatography (Mab Select Sure resin—General Electric). The expression yield was compared between the same bispecific antibody in the presence of the two different leader sequences—native leader and the mouse kappa leader. Similar expression yields (mg/L) were observed when expressing the same bispecific antibody with the different leaders. FIG. 5.

The functional impact of the N-terminal deletion may be due to steric hindrance from the side chain of the terminal serine residue, which could destabilise the disulphide bond between the light chain and heavy chain. It has been reported previously that deletion of an additional serine residue following the penultimate cysteine at the C-terminus of the λ Lc improved the thermal stability, stability to high pH, transient expression, purification yield, and ADCC function of IgG1 λ antibody (Shen et al., mAbs 5(3):418-431 2013). Similarly, a change in stability may underlie the effect on antibody function in the present case, although other explanations cannot be ruled out. In view of the impact observed in the FXase assay, which relies on antigen binding by both arms of the IgG, we could also attribute the change in activity to an effect on antigen binding by the binding site which is formed by the antibody Fv (paired VH and VL domains) of the antibody. There is possibly an effect on binding kinetics, e.g., off-rate and/or on-rate, potentially affecting the overall affinity (K_{D}) of the antibody-antigen interaction. Since this is a bispecific antibody, another possibility is that the presence vs absence of the N-terminal residue at IMGT position 1 of the VL domain changes the % heterodimer expressed by the cells. The heterodimer is the active bispecific molecule, while homodimers formed by pairing of identical heavy chains, represent a contaminant species. Inclusion of the N-terminal Ser might reduce % heterodimer, lowering the effective yield of bispecific antibody and thus reducing the activity observed from the antibody composition.

In the case of the anti-FIXxFX bispecific antibodies including the λ common light chain, the unexpected absence of the N-terminal Ser is an advantage, and we have demonstrated herein how this advantage may be secured with a variety of different expression systems, overcoming the inclusion of the N-terminal Ser when the λ light chain is expressed with alternative leader sequences, while maintaining antibody yield.

### Example 8 λ Antibody LARI

The human antibody designated LARI comprises an antigen-binding site provided within a VH domain and a λ VL domain. LARI was generated by immunisation of mice in which the heavy chain and λ light chain immunoglobulin loci had been engineered to contain human immunoglobulin heavy and λ light chain variable region gene segments, respectively. Comparison of the nucleotide sequence encoding the LARI VL domain with germline gene segments indicates that it was derived from recombination of human λ gene segments IGLV10-54 (allele Vλ10-54*d02) and IGLJ3*02 (allele Jλ3*02).

Vectors comprising DNA encoding λ antibody LARI were transfected into CHO cells. Three different leader sequences for the LARI VL domain were used—the native human Vλ10 family signal peptide (SEQ ID NO: 121), a murine signal peptide commonly referred to as the “Campath leader” (SEQ ID NO: 74) or a mouse kappa light chain signal peptide (SEQ ID NO: 62).

LARI IgG comprising the LARI VL domain with mouse kappa light chain signal peptide was expressed in CHO cells as described in Example 5.

LARI IgG comprising the LARI VL domain with native Vλ10 signal peptide or Campath leader peptide was expressed in CHO 3E7 cells. Briefly, 2.1×10⁶ cells/mL were used for each 2000 ml transfection and were incubated in a 37° C. incubator with a humidified atmosphere of 7% CO₂ in air on an orbital shaker rotating at 125-140 rpm for 1 hour.

As described in Example 3, MS was used to characterise and evaluate the molecular mass of the expressed antibody products.

When produced in cells expressing the light chain with the native Vλ10 family signal peptide, the molecular weight (MW) of the complete antibody was determined by MS to be smaller than the theoretical MW predicted by amino acid sequence of λ antibody LARI. Theoretical MW assumes that the light chain begins with IMGT residue 1 as the N terminal residue of the mature antibody. As shown in Table L, the amino acid sequence encoded by the germline gene segment Vλ10-54 begins:

1 2 3 4 5 6 7 8 9
Q A G L T Q P P S . . .

MS results indicated that the N-terminal glutamine and alanine residues were truncated when the native signal peptide was used. Thus, the LARI light chain expressed from its native signal peptide begins:

G L T Q P P S . . .

When the murine signal peptide SEQ ID NO: 74 (Campath leader) was used to express the light chain, the MW of the complete antibody was determined by MS to match the theoretical MW predicted by amino acid sequence of λ antibody LARI. MS results indicated that the N-terminal glutamine and alanine residues were intact when the murine signal peptide was used.

This reflects the findings observed with the 0128L antibody light chain in Example 3, i.e., the “textbook” sequence with IMGT position 1 as the first residue is obtained if the light chain of the human lambda antibody is expressed with a non-native signal peptide, whereas the (presumably natural) product of expression with the native λ signal peptide has an N-terminal truncation. Here, the λ VL domain was obtained from v gene segment vλ10-54 which is different in sequence from the v gene segment vλ3-21 (Table L), and the associated signal peptides for these v gene segments also differ (Table P). This indicates that this N-terminal clipping with expression from the native λ leader sequence is not limited to just the 0128L light chain, the vλ3-31 v gene segment or the vλ3 gene segment family.

To restore the natural N-terminus of the LARI light chain when expressed (e.g., in CHO cells) from a construct in which the LARI VL domain is preceded by a non-native leader sequence (such as the Campath leader), one may engineer the deletion of IMGT residues 1 and 2, i.e., the N-terminal glutamine and alanine residues of the VL domain. By fusing the non-native leader (either directly at the DNA level, or via an intron for the normal RNA splicing) to the N-terminus of the VL domain starting at IMGT position 3 (glycine), the LARI light chain will be cleaved from its signal peptide immediately before IMGT position 3.

Biological functions of LARI IgG expressed with these 2 different leader peptides showed no significant differences in in vitro assays. Nevertheless, it may be desirable to express the “native” VL domain (lacking IMGT residues 1 and 2) from the non-native leader, since this native VL domain N terminus is presumed to be the same as a human VL domain N terminus and is therefore less likely to be immunogenic when administered to humans.

When the mouse kappa light chain signal peptide SEQ ID NO: 62 was used to express the LARI light chain, 2 different MWs were identified in the recovered antibody product in a 3:2 ratio. One MW corresponded to the theoretical MW predicted by the amino acid sequence of λ antibody LARI, and the other corresponded to a smaller MW—in line with the truncation of the N-terminal glutamine and alanine residues. MS results indicated variable cleavage had occurred, accounting for 40% of truncated light chain in the recovered antibody product. Again, it would be possible to overcome this difficulty by engineering the VL domain N-terminus so that the protein expressed with this non-native signal peptide is the same as the protein expressed with its native signal peptide. For the LARI VL domain, this would involve deleting the encoded residues at IMGT positions 1 and 2 as explained above.

To conclude, the work presented in these Examples shows that:

(i) the sequence of the signal peptide can affect the position of cleavage of λ antibody light chains;

(ii) surprisingly, when a λ antibody light chain is expressed with its native signal peptide (e.g., a light chain derived from a human vλ3-21 gene segment expressed with the human vλ3 signal peptide, or a light chain derived from a human vλ10-54 gene segment expressed with the human vλ10 signal peptide), the signal peptide is cleaved to create an N-terminus which is not IMGT residue 1:

(iii) comparison of λ antibodies produced with native vs non-native signal peptides for the light chain identifies differences in molecular weight corresponding to absence of N-terminal residues in the former;

(iv) genetic engineering of the VL domain N terminus can be performed to avoid the difference in the mature polypeptide product expressed with the non-native leader compared with the mature polypeptide product expressed with the native leader. The sequence of the light chain (in particular, its N terminal sequence) can be engineered to be identical to that of the mature light chain sequence that is obtained after cleavage of the native leader. This is achievable by deletion of VL domain N-terminal residues which are absent when the light chain is expressed with its native leader. This engineered light chain sequence, when expressed with (and cleaved from) a non-native signal peptide, thus has an N terminal sequence identical to that which would be obtained from expression of the non-engineered light chain with (and its cleavage from) its native signal peptide.

### Tables

TABLE L

| Gene | Example | Accession | | | SEQ ID |
|---|---|---|---|---|---|
| segment | allele | number | Status | IMGT amino acid sequence | NO: |
| IGLV1-36 | IGLV1-36*01 | Z73653 | F | QSVLTQPPS.VSEAP RQRVTISCSGS | 1 |
| | | | | SSNI....GNNA VNWYQQLP | |
| | | | | GKAPKLLIY YD.......D LLPSGVS.D | |
| | | | | RFSGSK..SG TSASLAISGLQS | |
| | | | | EDEADYYC AAWDDSLNG | |
| IGLV1-40 | IGLVl-40*01 | M94116 | F | QSVLTQPPS.VSGAP GQRVTISCTGS | 2 |
| | | | | SSNIG...AGYD VHWYQQLP | |
| | | | | GTAPKLLIY GN.......S NRPSGVP.D | |
| | | | | RFSGSK..SG TSASLAITGLQA | |
| | | | | EDEADYYC QSYDSSLSG | |
| IGLV1-41 | IGLV1-41*01 | M94118 | ORF | QSVLTQPPS.VSAAP GQKVTISCSGS | 3 |
| | | | | SSDM....GNYA VSWYQQLP | |
| | | | | GTAPKLLIY EN.......N KRPSGIP.D | |
| | | | | RFSGSK..SG TSATLGITGLWP | |
| | | | | EDEADYYC LAWDTSPRA | |
| IGLV1-44 | IGLV1-44*01 | Z73654 | F | QSVLTQPPS.ASGTP GQRVTISCSGS | 4 |
| | | | | SSNI....GSNT VNWYQQLP | |
| | | | | GTAPKLLIY SN.......N QRPSGVP.D | |
| | | | | RFSGSK..SG TSASLAISGLQS | |
| | | | | EDEADYYC AAWDDSLNG | |
| IGLV1-47 | IGLV1-47*01 | Z73663 | F | QSVLTQPPS.ASGTP GQRVTISCSGS | 5 |
| | | | | SSNI .... GSNY VYWYQQLP | |
| | | | | GTAPKLLIY RN.......N QRPSGVP.D | |
| | | | | RFSGSK..SG TSASLAISGLRS | |
| | | | | EDEADYYC AAWDDSLSG | |
| IGLV1-50 | IGLVl-50*01 | M94112 | ORF | QSVLTQPPS.VSGAP GQRVTISCTGS | 6 |
| | | | | SSNIG...AGYV VHWYQQLP | |
| | | | | GTAPKLLIY GN.......S NRPSGVP.D | |
| | | | | QFSGSK..SG TSASLAITGLQS | |
| | | | | EDEADYYC KAWDNSLNA | |
| IGLV1-51 | IGLV1-51*01 | Z73661 | F | QSVLTQPPS. VSAAP GQKVTISCSGS | 7 |
| | | | | SSNI....GNNY VSWYQQLP | |
| | | | | GTAPKLLIY DN.......N KRPSGIP.D | |
| | | | | RFSGSK..SG TSATLGITGLQT | |
| | | | | GDEADYYC GTWDSSLSA | |
| IGLV1-62 | IGLV1-62*01 | D87022 | P | QSVLTQPPS.VSWAT RQRLTVSCTGS | 8 |
| | | | | SSNTG...TGYN VNCWQ*LP | |
| | | | | RTDPKLLRH GD.......K NWASWVS.D | |
| | | | | QFSGSK..SG SLASLGTTGLWA | |
| | | | | EDKTDYHC QSRDIC*VL | |
| IGLV2-5 | IGLV2-5*01 | Z73641 | P | QSALIQPPS.VSGSP GQSVTISCTGT | 9 |
| | | | | SSDVG...SYDY VSWYQQHP | |
| | | | | GTVPKPMIY NV.......N TQPSGVP.D | |
| | | | | RFSGSK..SG NTASMTISGLQA | |
| | | | | EDEADY*C CSYTSSAT* | |
| 1GLV2-8 | IGLV2-8*01 | X97462 | F | QSALTQPPS.ASGSP GQSVTISCTGT | 10 |
| | | | | SSDVG...GYNY VSWYQQHP | |
| | | | | GKAPKLMIY EV.......S KRPSGVP.D | |
| | | | | RFSGSK..SG NTASLTVSGLQA | |
| | | | | EDEADYYC SSYAGSNNF | |
| IGLV2-11 | IGLV2-ll*01 | Z73657 | F | QSALTQPRS.VSGSP GQSVTISCTGT | 11 |
| | | | | SSDVG...GYNY VSWYQQHP | |
| | | | | GKAPKLMIY DV.......S KRPSGVP.D | |
| | | | | RFSGSK..SG NTASLTISGLQA | |
| | | | | EDEADYYC CSYAGSYTF | |
| IGLV2-14 | IGLV2-14*01 | Z73664 | F | QSALTQPAS.VSGSP GQSITISCTGT | 12 |
| | | | | SSDVG...GYNY VSWYQQHP | |
| | | | | GKAPKLMIY EV.......S NRPSGVS.N | |
| | | | | RFSGSK..SG NTASLTISGLQA | |
| | | | | EDEADYYC SSYTSSSTL | |
| IGLV2-18 | IGLV2-18*01 | Z73642 | F | QSALTQPPS.VSGSP GQSVTISCTGT | 13 |
| | | | | SSDVG...SYNR VSWYQQPP | |
| | | | | GTAPKLMIY EV.......S NRPSGVP.D | |
| | | | | RFSGSK..SG NTASLTISGLQA | |
| | | | | EDEADYYC SLYTSSSTF | |
| IGLV2-23 | IGLV2-23*01 | X14616 | F | QSALTQPAS.VSGSP GQSITISCTGT | 14 |
| | | | | SSDVG...SYNL VSWYQQHP | |
| | | | | GKAPKLMIY EG.......S KRPSGVS.N | |
| | | | | RFSGSK..SG NTASLTISGLQA | |
| | | | | EDEADYYC CSYAGSSTL | |
| IGLV2-33 | IGLV2-33*01 | Z73643 | ORF | QSALTQPPF.VSGAP GQSVTISCTGT | 15 |
| | | | | SSDVG...DYDH VFWYQKRL | |
| | | | | STTSRLLIY NV.......N TRPSGIS.D | |
| | | | | LFSGSK..SG NMASLTISGLKS | |
| | | | | EVEANYHC SLYSSSYTF | |
| IGLV2-34 | IGLV2-34*01 | D87013 | P | QSVLTQPRS.VSRSP GQ*VTIFCTGT | 16 |
| | | | | SSDIG...GYDL VSWCQ*HP | |
| | | | | GKAPKLMIY DV.......A NWPSGAP.G | |
| | | | | CFSGSK..SG NTASLTISGLQA | |
| | | | | EDEADYYC SSYAGSYNF | |
| IGLV2-NL1 | IGLV2-NL1*01 | Z22209 | P | QSVLTQPRS.VSRSP GQ*VTIFCTGT | 17 |
| | | | | SSDIG...GYDL VSWCQ*HP | |
| | | | | GKAPKLMIY DV.......GNWPSGAP.G | |
| | | | | CFSGSK..SG NTASLTISGLQA | |
| | | | | EDEADYYC SSYAGSYNF | |
| IGLV3-1 | IGLV3-1*01 | X57826 | F | SYELTQPPS.VSVSP GQTASITCSGD | 18 |
| | | | | KLG......DKY ACWYQQKP | |
| | | | | GQSPVLVIY QD.......S KRPSGIP.E | |
| | | | | RFSGSN..SG NTATLTISGTQA | |
| | | | | MDEADYYC QAWDSSTA | |
| IGLV3-9 | IGLV3-9*01 | X97473 | F | SYELTQPLS.VSVAL GQTARITCGGN | 19 |
| | | | | NIG......SKN VHWYQQKP | |
| | | | | GQAPVLVIY RD.......S NRPSGIP.E | |
| | | | | RFSGSN..SG NTATLTISRAQA | |
| | | | | GDEADYYC QVWDSSTA | |
| IGLV3-10 | IGLV3-10*01 | X97464 | F | SYELTQPPS.VSVSP GQTARITCSGD | 20 |
| | | | | ALPKKY AYWYQQKS | |
| | | | | GQAPVLVIY ED.......S KRPSGIP.E | |
| | | | | RFSGSS..SG TMATLTISGAQV | |
| | | | | EDEADYYC YSTDSSGNH | |
| IGLV3-12 | IGLV3-12*01 | Z73658 | F | SYELTQPHS.VSVAT AQMARITCGGN | 21 |
| | | | | NIG......SKA VHWYQQKP | |
| | | | | GQDPVLVIY SD.......S NRPSGIP.E | |
| | | | | RFSGSN..PG NTTTLTISRIEA | |
| | | | | GDEADYYC QVWDSSSDH | |
| IGLV3-13 | IGLV3-13*01 | X97463 | P | SYELTQPPA.VSVSP GQTARISCSGD | 22 |
| | | | | VLR......DNY ADWYPQKP | |
| | | | | GQAPVLVIY KD.......G ERPSGIP.E | |
| | | | | RFSGST..SG NTTALTISRVLT | |
| | | | | KGGADYYC FSGD*NN | |
| IGLV3-16 | IGLV3-16*01 | X97471 | F | SYELTQPPS.VSVSL GQMARITCSGE | 23 |
| | | | | ALP......KKY AYWYQQKP | |
| | | | | GQFPVLVIY KD.......S ERPSGIP.E | |
| | | | | RFSGSS..SG TIVTLTISGVQA | |
| | | | | EDEADYYC LSADSSGTY | |
| IGLV3-19 | IGLV3-19*01 | X56178 | F | SSELTQDPA.VSVAL GQTVRITCQGD | 24 |
| | | | | SLR......SYY ASWYQQKP | |
| | | | | GQAPVLVIY GK.......N NRPSGIP.D | |
| | | | | RFSGSS..SG NTASLTITGAQA | |
| | | | | EDEADYYC NSRDSSGNH | |
| IGLV3-21 | IGLV3-21*01 | X71966 | F | SYVLTQPPS.VSVAP GKTARITCGGN | 25 |
| | | | | NIG......SKS VHWYQQKP | |
| | | | | GQAPVLVIY YD.......S DRPSGIP.E | |
| | | | | RFSGSN..SG NTATLTISRVEA | |
| | | | | GDEADYYC QVWDSSSDH | |
| IGLV3-22 | IGLV3-22*01 | Z73666 | F | SYELTQLPS.VSVSP GQTARITCSGD | 26 |
| | | | | VLG.......ENY ADWYQQKP | |
| | | | | GQAPELVIY ED.......S ERYPGIP.E | |
| | | | | RFSGST..SG NTTTLTISRVLT | |
| | | | | EDEADYYC LSGDEDN | |
| IGLV3-25 | IGLV3-25*01 | X97474 | F | SYELMQPPS.VSVSP GQTARITCSGD | 27 |
| | | | | ALPKQY AYWYQQKP | |
| | | | | GQAPVLVIY KDSERPSGIP.E | |
| | | | | RFSGSS..SG TTVTLTISGVQA | |
| | | | | EDEADYYC QSADSSGTY | |
| IGLV3-27 | IGLV3-27*01 | D86994 | F | SYELTQPSS.VSVSP GQTARITCSGD | 28 |
| | | | | VLA......KKY ARWFQQKP | |
| | | | | GQAPVLVIY KD.......S ERPSGIP.E | |
| | | | | RFSGSS..SG TTVTLTISGAQV | |
| | | | | EDEADYYC YSAADNN | |
| IGLV3-31 | IGLV3-31*01 | X97469 | P | SSELSQEPA.VSVAL G*TARITCQGD | 29 |
| | | | | SIE.......DSV VNWYKQKP | |
| | | | | SQAPGLVI* LN.......S VQSSGIP.K | |
| | | | | KFSGSS..SG NMATLTITGIQV | |
| | | | | EDKADYYC QSWDSSRTH | |
| IGLV3-32 | IGLV3-32*01 | Z73645 | ORF | SSGPTQVPA.VSVAL GQMARITCQGD | 30 |
| | | | | SME......GSY EHWYQQKP | |
| | | | | GQAPVLVIY DS.......S DRPSRIP.E | |
| | | | | RFSGSK..SG NTTTLTITGAQA | |
| | | | | EDEADYYY QLIDNHA | |
| IGLV4-3 | IGLV4-3*01 | X57828 | F | LPVLTQPPS.ASALL GASIKLTCTLS | 31 |
| | | | | SEHS.....TYT IEWYQQRP | |
| | | | | GRSPQYIMK VKSD...GSH SKGDGIP.D | |
| | | | | RFMGSS..SG ADRYLTFSNLQS | |
| | | | | DDEAEYHC GESHTIDGQVG* | |
| IGLV4-60 | IGLV4-60*01 | Z73667 | F | QPVLTQSSS.ASASL GSSVKLTCTLS | 32 |
| | | | | SGHS.....SYI IAWHQQQP | |
| | | | | GKAPRYLMK LEGS...GSY NKGSGVP.D | |
| | | | | RFSGSS..SG ADRYLTISNLQL | |
| | | | | EDEADYYC ETWDSNT | |
| IGLV4-69 | IGLV4-69*01 | Z73648 | F | QLVLTQSPS.ASASL GASVKLTCTLS | 33 |
| | | | | SGHS.....SYA IAWHQQQP | |
| | | | | EKGPRYLMK LNSD...GSH SKGDGIP.D | |
| | | | | RFSGSS..SG AERYLTISSLQS | |
| | | | | EDEADYYC QTWGTGI | |
| IGLV5-37 | IGLV5-37*01 | Z73672 | F | QPVLTQPPS.SSASP GESARLTCTLP | 34 |
| | | | | SDINV... GSYN IYWYQQKP | |
| | | | | GSPPRYLLY YYSD...SDK GQGSGVP.S | |
| | | | | RFSGSKDASA NTGILLISGLQS | |
| | | | | EDEADYYC MIWPSNAS | |
| IGLV5-39 | IGLV5-39*01 | Z73668 | F | QPVLTQPTS.LSASP GASARFTCTLR | 35 |
| | | | | SGINV...GTYR IYWYQQKP | |
| | | | | GSLPRYLLR YKSD...SDK QQGSGVP.S | |
| | | | | RFSGSKDAST NAGLLLISGLQS | |
| | | | | EDEADYYC AIWYSSTS | |
| IGLV5-45 | IGLV5-45*01 | 273670 | F | QAVLTQPAS.LSASP GASASLTCTLR | 36 |
| | | | | SGINV...GTYR IYWYQQKP | |
| | | | | GSPPQYLLR YKSD...SDK QQGSGVP.S | |
| | | | | RFSGSKDASA NAGILLISGLQS | |
| | | | | EDEADYYC MIWHSSAS | |
| IGLV5-48 | IGLV5-48*01 | Z73649 | ORF | QPVLTQPTS.LSASP GASARLTCTLR | 37 |
| | | | | SGINL...GSYR IFWYQQKP | |
| | | | | ESPPRYLLS YYSD...SSK HQGSGVP.S | |
| | | | | RFSGSKDASS NAGILVISGLQS | |
| | | | | EDEADYYC MIWHSSAS | |
| IGLV5-52 | IGLV5-52*01 | Z73669 | F | QPVLTQPSS.HSASS GASVRLTCMLS | 38 |
| | | | | SGFSV...GDFW IRWYQQKP | |
| | | | | GNPPRYLLY YHSD...SNK GQGSGVP.S | |
| | | | | RFSGSNDASA NAGILRISGLQP | |
| | | | | EDEADYYC GTWHSNSKT | |
| IGLV6-57 | IGLV6-57*01 | Z73673 | F | NFMLTQPHS.VSESP GKTVTISCTRS | 39 |
| | | | | SGSI....ASNY VQWYQQRP | |
| | | | | GSSPTTVIY ED.......N QRPSGVP.D | |
| | | | | RFSGSIDSSS NSASLTISGLKT | |
| | | | | EDEADYYC QSYDSSN | |
| IGLV7-43 | IGLV7-43*01 | X14614 | F | QTWTQEPS . LTVSP GGTVTLTCASS | 40 |
| | | | | TGAVT...SGYY PNWFQQKP | |
| | | | | GQAPRALIY ST.......S NKHSWTP.A | |
| | | | | RFSGSL..LG GKAALTLSGVQP | |
| | | | | EDEAEYYC LLYYGGAQ | |
| IGLV7-46 | IGLV7-46*01 | Z73674 | F | QAWTQEPS. LTVSP GGTVTLTCGSS | 41 |
| | | | | TGAVT...SGHY PYWFQQKP | |
| | | | | GQAPRTLIY DT.......S NKHSWTP.A | |
| | | | | RFSGSL..LG GKAALTLSGAQP | |
| | | | | EDEAEYYC LLSYSGAR | |
| IGLV8-61 | IGLV8-61*01 | 273650 | F | QTWTQEPS. FSVSP GGTVTLTCGLS | 42 |
| | | | | SGSVS...TSYY PSWYQQTP | |
| | | | | GQAPRTLIY ST.......N TRSSGVP.D | |
| | | | | RFSGSI..LG NKAALTITGAQA | |
| | | | | DDESDYYC VLYMGSGI | |
| IGLV9-49 | IGLV9-49*01 | Z73675 | F | QPVLTQPPS.ASASL GASVTLTCTLS | 43 |
| | | | | SGYS.....NYK VDWYQQRP | |
| | | | | GKGPRFVMR VGTG..GIVG SKGDGIP.D | |
| | | | | RFSVLG..SG LNRYLTIKNIQE | |
| | | | | EDESDYHC GADHGSGSNFV* | |
| IGLV10-54 | IGLV10-54*01 | Z73676 | F | QAGLTQPPS.VSKGL RQTATLTCTGN | 44 |
| | | | | SNNV....GNQG AAWLQQHQ | |
| | | | | GHPPKLLSY RN.......NNRPSGIS.E | |
| | | | | RLSASR..SG NTASLTITGLQP | |
| | | | | EDEADYYC SAWDSSLSA | |
| IGLV11-55 | IGLV11-55*01 | D86996 | ORF | RPVLTQPPS.LSASP GATARLPCTLS | 45 |
| | | | | SDLSV...GGKN MFWYQQKP | |
| | | | | GSSPRLFLY HYSD...SDK QLGPGVP.S | |
| | | | | RVSGSKETSS NTAFLLISGLQP | |
| | | | | EDEADYYC QVYESSAN | |

TABLE L
Human v lambda alleles and sequences from IMGT
database. Only the *01 allele of each segment
is shown. Dots (.) indicate gaps according to
the IMGT unique numbering as defined by Lefranc,
M.-P. et al., Dev. Comp. Immunol., 27, 55-77
(2003). Asterisk (*) in the sequence indicate
a stop codon. F: Functional; P: Pseudogene;
ORF: Open Reading Frame as defined by IMGT.
Translation of each germline v gene segment
is grouped into 11 amino acid sequence blocks,
which correspond to the following components
of the VL domain, respectively (shown for
IGLV1-36*01 sequence example):

| | | | |
|---|---|---|---|
| QSVLTQPPS.VSEAP | FR1 | A | IMGT 1-15 |
| RQRVTISCSGS | FR1 | B | IMGT 16-26 |
| SSNI....GNNA | CDR1 | B-C loop | IMGT 27-38 |
| VNWYQQLP | FR2 | C | IMGT 39-46 |
| GKAPKLLIY | FR2 | C | IMGT 47-55 |
| YD.......D | CDR2 | C′-C″ loop | IMGT 56-65 |
| LLPSGVS.D | FR3 | C″ | IMGT 66-74 |
| RFSGSK..SG | FR3 | D | IMGT 75-85 |
| TSASLAISGLQS | FR3 | E | IMGT 85-96 |
| EDEADYYC | FR3 | F | IMGT 97-104 |
| AAWDDSLNG | CDR3 | F-G loop | IMGT 105-117 |
| | | | (j segment |
| | | | sequence not |
| | | | shown) |

On recombination to generate the VL domain, the v segment combines with the downstream j segment and the LCDR3 comprises the v-j junction.

TABLE N
Nucleotide sequences of known human vλ3-21 alleles.

| IGLV3-21 | |
|---|---|
| allele | Nucleotide sequence |
| vλ3-21*d01 | TCCTATGTGCTGACTCAGCCACCCTCAGTGTCAGTGGCCCCAGGAAAGACGGCCAGGATTACCTGTGGGGG |
| SEQ ID NO: | AAACAACATTGGAAGTAAAAGTGTGCACTGGTACCAGCAGAAGCCAGGCCAGGCCCCTGTGCTGGTCATCT |
| 56 | ATTATGATAGCGACCGGCCCTCAGGGATCCCTGAGCGATTCTCTGGCTCCAACTCTGGGAACACGGCCACC |
| | CTGACCATCAGCAGGGTCGAAGCCGGGGATGAGGCCGACTATTACTGTCAGGTGTGGGATAGTAGTAGTGA |
| | TCATCC |
| vλ3-21*01 | TCCTATGTGCTGACTCAGCCACCCTCAGTGTCAGTGGCCCCAGGAAAGACGGCCAGGATTACCTGTGGGGG |
| X71966 | AAACAACATTGGAAGTAAAAGTGTGCACTGGTACCAGCAGAAGCCAGGCCAGGCCCCTGTGCTGGTCATCT |
| SEQ ID NO: | ATTATGATAGCGACCGGCCCTCAGGGATCCCTGAGCGATTCTCTGGCTCCAACTCTGGGAACACGGCCACC |
| 57 | CTGACCATCAGCAGGGTCGAAGCCGGGGATGAGGCCGACTATTACTGTCAGGTGTGGGACAGTAGTAGTGA |
| | TCATCC |
| vλ3-21*02 | TCCTATGTGCTGACTCAGCCACCCTCGGTGTCAGTGGCCCCAGGACAGACGGCCAGGATTACCTGTGGGGG |
| D87007 | AAACAACATTGGAAGTAAAAGTGTGCACTGGTACCAGCAGAAGCCAGGCCAGGCCCCTGTGCTGGTCGTCT |
| SEQ ID NO: | ATGATGATAGCGACCGGCCCTCAGGGATCCCTGAGCGATTCTCTGGCTCCAACTCTGGGAACACGGCCACC |
| 58 | CTGACCATCAGCAGGGTCGAAGCCGGGGATGAGGCCGACTATTACTGTCAGGTGTGGGATAGTAGTAGTGA |
| | TCATCC |
| vλ3-21*03 | TCCTATGTGCTGACTCAGCCACCCTCGGTGTCAGTGGCCCCAGGAAAGACGGCCAGGATTACCTGTGGGGG |
| M94115 | AAACAACATTGGAAGTAAAAGTGTGCACTGGTACCAGCAGAAGCCAGGCCAGGCCCCTGTGCTGGTCGTCT |
| SEQ ID NO: | ATGATGATAGCGACCGGCCCTCAGGGATCCCTGAGCGATTCTCTGGCTCCAACTCTGGGAACACGGCCACC |
| 59 | CTGACCATCAGCAGGGTCGAAGCCGGGGATGAGGCCGACTATTACTGTCAGGTGTGGGATAGTAGTAGTGA |
| | TCATCC |

TABLE P
Examples of signal peptides

| SEQ ID | Name | Sequence | Reference |
|---|---|---|---|
| 60 | human signal peptide | MAWTALLLGLLSHCTGSVT | |
| | IGLV3-21, native for | | |
| | 0128L and 0325L VL | | |
| | domains | | |
| 61 | germline nucleotide | ATGGCCTGGACCGCTCTCCTCCTCGGCCTCCTCTCTC | |
| | sequence encoding human | ACTGCACAGGCTCTGTGACC | |
| | signal peptide IGLV3-21 | | |
| 62 | mouse kappa light chain | MSVPTQVLGLLLLWLTDARC | Whittle et al., Protein |
| | signal peptide | | Engineering 1(6): 499- |
| | | | 505 1987 |
| 63 | germline nucleotide | ATGAGTGTGCCCACTCAGGTCCTGGGGTTGCTGCTGC | Whittle et al., Protein |
| | sequence encoding mouse | TGTGGCTTACAGATGCCAGATGT | Engineering 1(6): 499- |
| | kappa light chain signal | | 505 1987 |
| | peptide | | |
| 64 | nucleotide sequence | ATGTCTGTGCCTACACAGGTTCTGGGACTGCTGCTGC | |
| | encoding mouse kappa | TGTGGCTGACCGACGCCAGATGT | |
| | light chain signal | | |
| | peptide, codon- | | |
| | optimised for CHO | | |
| 65 | nucleic acid encoding | ATGTCTGTGCCTACACAGGTTCTGGGACTGCTGCTGCTGTGGCTGACCGACGCCAGATGTTAC | |
| | 0325L VL domain with | GTGCTGACCCAGCCTCCTTCCGTGTCTGTTGCTCCTGGCGAGACAGCCAGAATCACCTGTGGC | |
| | signal peptide | GGCGATAACATCGGCCGGAAGTCCGTGTACTGGTATCAGCAGAAGTCCGGCCAGGCTCCTGTG | |
| | | CTGGTCATCTACTACGACTCCGACCGGCCTTCTGGCATCCCTGAGAGATTCTCCGGCTCCAAC | |
| | | TCCGGCAATACCGCCACACTGACCATCTCCAGAGTGGAAGCTGGCGACGAGGCCGACTACTAC | |
| | | TGCCAAGTGTGGGACGGCTCCTCTGACCACTGGGTTTTCGGCGGAGGCACCAAGCTGACAGTG | |
| | | CTG | |
| 66 | 0325L with mouse kappa | MSVPTQVLGLLLLWLTDARCYVLTQPPSVSVAPGETARITCGGDNIGRKSVYWYQQKSGQAPV | |
| | signal peptide | LVIYYDSDRPSGIPERFSGSNSGNTATLTISRVEAGDEADYYCQVWDGSSDHWVFGGGTKLTV | |
| | | L | |
| 67 | A light chain comprising | MSVPTQVLGLLLLWLTDARCYVLTQPPSVSVAPGETARITCGGDNIGRKSVYWYQQKSGQAPV | |
| | 0325L VL domain with mouse | LVIYYDSDRPSGIPERFSGSNSGNTATLTISRVEAGDEADYYCQVWDGSSDHWVFGGGTKLTV | |
| | kappa signal peptide | LGQPKAAPSVTLFPPSSEELQANKATLVCLISDFYPGAVTVAWKADSSPVKAGVETTTPSKQS | |
| | | NNKYAASSYLSLTPEQWKSHKSYSCQVTHEGSTVEKTVAPTECS | |
| 68 | 0128L VL domain with mouse | MSVPTQVLGLLLLWLTDARCSYVLTQPPSVSVAPGETARITCGGDNIGRKSVYWYQQKSGQAP | |
| | kappa signal peptide | VLVIYYDSDRPSGIPERFSGSNSGNTATLTISRVEAGDEADYYCQVWDGSSDHWVFGGGTKLT | |
| | | VL | |
| 69 | V12 signal peptide derived | MRFFFVFLAIVLFQGIHG | WO2008/148519 |
| | from Sandfly Yellow related | | |
| | protein | | |
| 70 | V14 signal peptide derived | MKPIFLVLLWT SAYA | WO2008/148519 |
| | from Silkworm Fibroin LC | | |
| 71 | V16 signal peptide derived | MRTLWIMAVLLLGVEG | WO2008/148519 |
| | from Snake PLA2 | | |
| 72 | V17 signal peptide derived | MKTLILAVALVYCATVHC | WO2008/148519 |
| | from Cypridina Noctiluca | | |
| | luciferase | | |
| 73 | V19 signal peptide derived | MMRPIVLVLLFATSALA | WO2008/148519 |
| | from Pinemoth Fibroin LC | | |
| 74 | murine signal peptide | MGWSCIILFLVATATGVHS | Gibson et al 2017 |
| | (Campath) | | |
| 75 | V lambda 3 family signal | MAWTPLLLPLLTLCTSEA | Gibson et al 2017 |
| | peptide | | |
| 76 | V lambda 1 family signal | MAGFPLLLTLLTHCAGSWA | Gibson et al 2017 |
| | peptide | | |
| 77 | V kappa 1 family signal | MDMRVPAQLLGLLLLWLPGAKC | Gibson et al 2017 |
| | peptide | | |
| 121 | V lambda 10 family signal | MPWALLLLTLLTHSAVSW | |
| | peptide | | |

TABLE S
Sequences of antibody heavy and light chain domains, all of which represent embodiments of the
present invention.

| | | |
|---|---|---|
| 78 | N1280H VH domain | GAGGTGCAGCTGGTGGAGTCTGGGGGAGGCTTTGTCCAGCCTGGGGGGTCCCTGAGACTCTCCTGTGCAGTCTCTGGAT |
| | coding nucleotide | TCAGATTTAATAGCTATTGGATGAGCTGGGTCCGCCAGGCTCCAGGGAAGGGGCTGGAGTGGGTGGCCAACATAAACCA |
| | sequence | AGATGGAAGTAGAAAATTCTATGTGGCCTCTGTGAAGGGCCGATTCACCATGTCCAGAGACAACGCCAAGAAATCAGTG |
| | | TATGTACAAATGAACAGCCTGAGAGCCGAGGACACGGCTGTGTATTACTGTGCGAGAGAGGGGTATAGTAGTATCAAGT |
| | | ATTATGGTATGGACGTCTGGGGCCAAGGGACCACGGTCACCGTCTCCTCA |
| 79 | N1280H VH domain | EVQLVESGGGFVQPGGSLRLSCAVSGFRFNSYWMSWVRQAPGKGLEWVANINQDGSRKFYVASVKGRFTMSRDNAKKSV |
| | amino acid | YVQMNSLRAEDTAVYYCAREGYSSIKYYGMDVWGQGTTVTVSS |
| | sequence | |
| 80 | Nucleic acid | GAAGTGCAGCTGGTTGAATCTGGCGGCGGATTTGTTCAGCCTGGCGGCTCTCTGAGACTGTCCTGTGCTGTGTCCGGCT |
| | encoding N1280H- | TCCGGTTCAACTCCTACTGGATGTCCTGGGTCCGACAGGCTCCTGGCAAAGGACTGGAATGGGTCGCCAACATCAACCA |
| | IgG4-P K439E | GGACGGCTCCCGGAAGTTCTACGTGGCCTCTGTGAAGGGCAGATTCACCATGTCTCGGGACAACGCCAAGAAATCCGTG |
| | heavy chain; | TACGTGCAGATGAACTCCCTGAGAGCCGAGGACACCGCCGTGTACTACTGTGCTAGAGAGGGCTACTCCTCCATCAAGT |
| | N1280 coding | ACTACGGCATGGACGTGTGGGGCCAGGGCACAACCGTGACAGTCTCTTCCGCTTCCACCAAGGGACCCAGCGTTTTCCC |
| | sequence | TCTGGCTCCTTGCTCCAGATCCACCTCCGAGTCTACAGCTGCTCTGGGCTGCCTGGTCAAGGACTACTTTCCTGAGCCT |
| | underlined. | GTGACCGTGTCCTGGAACTCTGGCGCTCTGACATCTGGCGTGCACACCTTTCCAGCTGTGCTGCAGTCCTCCGGCCTGT |
| | | ACTCTCTGTCCTCTGTCGTGACCGTGCCTTCCAGCTCTCTGGGAACCCAGACCTACACCTGTAATGTGGACCACAAGCC |
| | | TTCCAACACCAAGGTGGACAAGCGCGTGGAATCTAAGTACGGCCCTCCTTGTCCTCCATGTCCTGCTCCAGAGTTTCTC |
| | | GGCGGACCCTCCGTGTTTCTGTTCCCTCCAAAGCCTAAGGACACCCTGATGATCTCTCGGACCCCTGAAGTGACCTGCG |
| | | TGGTGGTGGATGTGTCCCAAGAGGATCCCGAGGTGCAGTTCAATTGGTACGTGGACGGCGTGGAAGTGCACAATGCCAA |
| | | GACCAAGCCTAGAGAGGAACAGTACAACTCCACCTACAGAGTGGTGTCCGTGCTGACCGTGCTGCACCAGGATTGGCTG |
| | | AACGGCAAAGAGTACAAGTGCAAGGTGTCCAACAAGGGCCTGCCTAGCTCCATCGAAAAGACCATCTCCAAGGCCAAGG |
| | | GCCAGCCTCGAGAACCCCAGGTTTACACCCTGCCTCCAAGCCAAGAGGAAATGACCAAGAACCAGGTGTCCCTGACCTG |
| | | TCTCGTGAAGGGCTTCTACCCCTCCGATATCGCCGTGGAATGGGAGTCTAATGGCCAGCCAGAGAACAACTACAAGACC |
| | | ACACCTCCAGTGCTGGACTCCGACGGCTCATTCTTTCTGTACTCCAAGCTGACAGTGGACAAGTCCCGGTGGCAAGAGG |
| | | GCAACGTGTTCTCCTGCTCTGTGATGCACGAGGCCCTGCACAACCACTACACCCAAGAGTCCCTGTCTCTGTCCCCT |
| 81 | N1280H-IgG4-P | EVQLVESGGGFVQPGGSLRLSCAVSGFRFNSYWMSWVRQAPGKGLEWVANINQDGSRKFYVASVKGRFTMSRDNAKKSV |
| | K439E amino acid | YVQMNSLRAEDTAVYYCAREGYSSIKYYGMDVWGQGTTVTVSSASTKGPSVFPLAPCSRSTSESTAALGCLVKDYFPEP |
| | sequence | VTVSWNSGALTSGVHTFPAVLQSSGLYSLSSVVTVPSSSLGTQTYTCNVDHKPSNTKVDKRVESKYGPPCPPCPAPEFL |
| | | GGPSVFLFPPKPKDTLMISRTPEVTCVVVDVSQEDPEVQFNWYVDGVEVHNAKTKPREEQYNSTYRVVSVLTVLHQDWL |
| | | NGKEYKCKVSNKGLPSSIEKTISKAKGQPREPQVYTLPPSQEEMTKNQVSLTCLVKGFYPSDIAVEWESNGQPENNYKT |
| | | TPPVLDSDGSFFLYSKLTVDKSRWQEGNVFSCSVMHEALHNHYTQESLSLSP |
| 82 | N1441H VH domain | GAGGTGCAGCTGGTTGAATCTGGCGGCGGATTTGTTCAGCCTGGCGGCTCTCTGAGACTGAGCTGTGCCGTGTCCGGCT |
| | encoding | TCCGGTTCAACAGCTACTGGATGTCCTGGGTCCGACAGGCCCCTGGCAAAGGACTTGAGTGGGTCGCCAACATCAACCA |
| | nucleotide | GGACGGCAGCCGGAAGTTTTACGTGGCCTCTGTGAAGGGCAGATTCACCATGAGCCGGGACAACGCCGACAAAAGCGTG |
| | sequence | TACGTGCAGATGAACAGCCTGAGAGCCGAGGACACCGCCGTGTACTATTGTGCCAGAGAGGGCTACAGCAGCATCAAGT |
| | | ACTACGGCATGGACGTGTGGGGCCAGGGCACAACAGTGACAGTCTCTTCT |
| 83 | N1441H VH domain | EVQLVESGGGFVQPGGSLRLSCAVSGFRFNSYWMSWVRQAPGKGLEWVANINQDGSRKFYVASVKGRFTMSRDNADKSV |
| | amino acid | YVQMNSLRAEDTAVYYCAREGYSSIKYYGMDVWGQGTTVTVSS |
| | sequence | |
| 84 | Nucleic acid | GAAGTGCAGCTGGTTGAATCTGGCGGCGGATTTGTTCAGCCTGGCGGCTCTCTGAGACTGTCCTGTGCTGTGTCCGGCT |
| | encoding N1441H- | TCCGGTTCAACTCCTACTGGATGTCCTGGGTCCGACAGGCTCCTGGCAAAGGACTGGAATGGGTCGCCAACATCAACCA |
| | IgG4-P K439E | GGACGGCTCCCGGAAGTTCTACGTGGCCTCTGTGAAGGGCAGATTCACCATGTCTCGGGACAACGCCGACAAGTCCGTG |
| | heavy chain | TACGTGCAGATGAACTCCCTGAGAGCCGAGGACACCGCCGTGTACTACTGTGCTAGAGAGGGCTACTCCTCCATCAAGT |
| | | ACTACGGCATGGACGTGTGGGGCCAGGGCACAACCGTGACAGTCTCTTCCGCTTCCACCAAGGGACCCAGCGTTTTCCC |
| | | TCTGGCTCCTTGCTCCAGATCCACCTCCGAGTCTACAGCTGCTCTGGGCTGCCTGGTCAAGGACTACTTTCCTGAGCCT |
| | | GTGACCGTGTCCTGGAACTCTGGCGCTCTGACATCTGGCGTGCACACCTTTCCAGCTGTGCTGCAGTCCTCCGGCCTGT |
| | | ACTCTCTGTCCTCTGTCGTGACCGTGCCTTCCAGCTCTCTGGGAACCCAGACCTACACCTGTAATGTGGACCACAAGCC |
| | | TTCCAACACCAAGGTGGACAAGCGCGTGGAATCTAAGTACGGCCCTCCTTGTCCTCCATGTCCTGCTCCAGAGTTTCTC |
| | | GGCGGACCCTCCGTGTTTCTGTTCCCTCCAAAGCCTAAGGACACCCTGATGATCTCTCGGACCCCTGAAGTGACCTGCG |
| | | TGGTGGTGGATGTGTCCCAAGAGGATCCCGAGGTGCAGTTCAATTGGTACGTGGACGGCGTGGAAGTGCACAACGCCAA |
| | | GACCAAGCCTAGAGAGGAACAGTACAACTCCACCTACAGAGTGGTGTCCGTGCTGACCGTGCTGCACCAGGATTGGCTG |
| | | AACGGCAAAGAGTACAAGTGCAAGGTGTCCAACAAGGGCCTGCCTAGCTCCATCGAAAAGACCATCTCCAAGGCCAAGG |
| | | GCCAGCCTCGAGAACCCCAGGTTTACACCCTGCCTCCAAGCCAAGAGGAAATGACCAAGAACCAGGTGTCCCTGACCTG |
| | | TCTCGTGAAGGGCTTCTACCCCTCCGATATCGCCGTGGAATGGGAGTCTAATGGCCAGCCAGAGAACAACTACAAGACC |
| | | ACACCTCCAGTGCTGGACTCCGACGGCTCATTCTTTCTGTACTCCAAGCTGACAGTGGACAAGTCCCGGTGGCAAGAGG |
| | | GCAACGTGTTCTCCTGCTCTGTGATGCACGAGGCCCTGCACAACCACTACACCCAAGAGTCCCTGTCTCTGTCCCCT |
| 85 | N1441H-IgG4-P | EVQLVESGGGFVQPGGSLRLSCAVSGFRFNSYWMSWVRQAPGKGLEWVANINQDGSRKFYVASVKGRFTMSRDNADKSV |
| | K439E amino acid | YVQMNSLRAEDTAVYYCAREGYSSIKYYGMDVWGQGTTVTVSSASTKGPSVFPLAPCSRSTSESTAALGCLVKDYFPEP |
| | sequence | VTVSWNSGALTSGVHTFPAVLQSSGLYSLSSVVTVPSSSLGTQTYTCNVDHKPSNTKVDKRVESKYGPPCPPCPAPEFL |
| | | GGPSVFLFPPKPKDTLMISRTPEVTCVVVDVSQEDPEVQFNWYVDGVEVHNAKTKPREEQYNSTYRVVSVLTVLHQDWL |
| | | NGKEYKCKVSNKGLPSSIEKTISKAKGQPREPQVYTLPPSQEEMTKNQVSLTCLVKGFYPSDIAVEWESNGQPENNYKT |
| | | TPPVLDSDGSFFLYSKLTVDKSRWQEGNVFSCSVMHEALHNHYTQESLSLSP |
| 86 | T0999H VH domain | CAGGTTCAGCTGATTCAGTCCGGCGCCAAAGTGAAGAAACCTGGCGCCTCTGTGAAGGTGTCCTGCAAGGCCTCTCGGT |
| | coding nucleotide | ACAAGTTCACCTCCTACTACATGCACTGGGTCCGACAGGCCCCTGGACAAGGATTGGAGTGGATGGGCATCATCAACCC |
| | sequence | CAAGTCCGGCTCCACCTCTTACGCCCAGAAATTCCAGGGCAGAGTGACCATGACCAGAGACACCTCTACCTCCACCGTG |
| | | TACATGGAACTGTCCAGCCTGAGATCCGAGGACACCGCCGTGTACTACTGTGCCAGAGATGGCTACGGCAGCTTCTCCA |
| | | GACTGATCCAGTTGTGGGGCCAGGGCACACTGGTCACAGTGTCCTCT |
| 87 | T0999H VH domain | QVQLIQSGAKVKKPGASVKVSCKASRYKFTSYYMHWVRQAPGQGLEWMGIINPKSGSTSYAQKFQGRVTMTRDTSTSTV |
| | amino acid | YMELSSLRSEDTAVYYCARDGYGSFSRLIQLWGQGTLVTVSS |
| | sequence | |
| 88 | Nucleic acid | CAGGTTCAGCTGATTCAGTCCGGCGCCAAAGTGAAGAAACCTGGCGCCTCTGTGAAGGTGTCCTGCAAGGCCTCTCGGT |
| | encoding T0999H- | ACAAGTTCACCTCCTACTACATGCACTGGGTCCGACAGGCCCCTGGACAAGGATTGGAGTGGATGGGCATCATCAACCC |
| | IgG4-P E356K | CAAGTCCGGCTCCACCTCTTACGCCCAGAAATTCCAGGGCAGAGTGACCATGACCAGAGACACCTCTACCTCCACCGTG |
| | heavy chain; | TACATGGAACTGTCCAGCCTGAGATCCGAGGACACCGCCGTGTACTACTGTGCCAGAGATGGCTACGGCAGCTTCTCCA |
| | T0999H coding | GACTGATCCAGTTGTGGGGCCAGGGCACACTGGTCACAGTGTCCTCTGCTTCCACCAAGGGACCCAGCGTGTTCCCTCT |
| | sequence | GGCTCCTTGCTCCAGATCCACCTCCGAGTCTACAGCTGCTCTGGGCTGCCTGGTCAAGGACTACTTTCCTGAGCCTGTG |
| | underlined | ACCGTGTCTTGGAACTCTGGCGCTCTGACATCTGGCGTGCACACCTTTCCAGCTGTGCTGCAGTCCTCCGGCCTGTACT |
| | | CTCTGTCCTCTGTCGTGACCGTGCCTTCCAGCTCTCTGGGAACCCAGACCTACACCTGTAATGTGGACCACAAGCCTTC |
| | | CAACACCAAGGTGGACAAGCGCGTGGAATCTAAGTACGGCCCTCCTTGTCCTCCATGTCCTGCTCCAGAGTTTCTCGGC |
| | | GGACCCTCCGTGTTTCTGTTCCCTCCAAAGCCTAAGGACACCCTGATGATCTCTCGGACCCCTGAAGTGACCTGCGTGG |
| | | TGGTGGATGTGTCCCAAGAGGATCCCGAGGTGCAGTTCAATTGGTACGTGGACGGCGTGGAAGTGCACAACGCCAAGAC |
| | | GGCAAAGAGTACAAGTGCAAGGTGTCCAACAAGGGCCTGCCTAGCTCCATCGAAAAGACCATCTCCAAGGCCAAGGGCC |
| | | AGCCTCGAGAACCCCAGGTTTACACCCTGCCTCCAAGCCAGAAAGAGATGACCAAGAACCAGGTGTCCCTGACCTGCCT |
| | | CGTGAAGGGCTTCTACCCTTCCGATATCGCCGTGGAATGGGAGAGCAATGGCCAGCCAGAGAACAACTACAAGACCACA |
| | | CCTCCTGTGCTGGACTCCGATGGCTCATTCTTTCTGTACTCCAAGCTGACAGTGGACAAGTCCCGGTGGCAAGAGGGCA |
| | | ACGTGTTCTCCTGCTCTGTGATGCACGAGGCCCTGCACAACCACTACACCCAGAAGTCCCTGTCTCTGTCCCCT |
| 89 | T0999H-IgG4-P | QVQLIQSGAKVKKPGASVKVSCKASRYKFTSYYMHWVRQAPGQGLEWMGIINPKSGSTSYAQKFQGRVTMTRDTSTSTV |
| | E356K amino acid | YMELSSLRSEDTAVYYCARDGYGSFSRLIQLWGQGTLVTVSSASTKGPSVFPLAPCSRSTSESTAALGCLVKDYFPEPV |
| | sequence | TVSWNSGALTSGVHTFPAVLQSSGLYSLSSVVTVPSSSLGTQTYTCNVDHKPSNTKVDKRVESKYGPPCPPCPAPEFLG |
| | | GPSVFLFPPKPKDTLMISRTPEVTCVVVDVSQEDPEVQFNWYVDGVEVHNAKTKPREEQYNSTYRVVSVLTVLHQDWLN |
| | | GKEYKCKVSNKGLPSSIEKTISKAKGQPREPQVYTLPPSQKEMTKNQVSLTCLVKGFYPSDIAVEWESNGQPENNYKTT |
| | | PPVLDSDGSFFLYSKLTVDKSRWQEGNVFSCSVMHEALHNHYTQKSLSLSP |
| 90 | T0201H VH | CAGGTTCAGCTGATTCAGTCCGGCGCCAAAGTGAAGAAACCTGGCGCCTCTGTGAAGGTGTCCTGCAAGGCCTCTCGGT |
| | sequence | ACAGCTTCACCAGCTACTATATGCACTGGGTGCGACAGGCCCCTGGACAAGGGCTTGAGTGGATGGGAATAATCAACCC |
| | | TAAAAGTGGTAGTACAAGTTACGCACAGAAGTTCCAGGGCAGAGTCACCATGACCAGGGACACGTCCACGAGCACAGTC |
| | | TACATGGAGCTGAGCAGCCTGAGATCTGAGGACACGGCCGTGTATTACTGTGCGAGAGATGGGTATGGCAGCTCGTCCC |
| | | GGTGCCTCCAGCTCTGGGGCCAGGGCACCCTGGTCACCGTCTCCTCA |
| 91 | T0201H VH | QVQLIQSGAKVKKPGASVKVSCKASRYKFTSYYMHWVRQAPGQGLEWMGIINPKSGSTSYAQKFQGRVTMTRDTSTSTV |
| | | YMELSSLRSEDTAVYYCARDGYGSSSRCLQLWGQGTLVTVSS |
| 92 | T0736H VH | CAGGTTCAGCTGATTCAGTCCGGCGCCAAAGTGAAGAAACCTGGCGCCTCTGTGAAGGTGTCCTGCAAGGCCTCTCGGT |
| | sequence | ACAAGTTCACCAGCTACTATATGCACTGGGTGCGACAGGCCCCTGGACAAGGGCTTGAGTGGATGGGAATAATCAACCC |
| | | TAAAAGTGGTAGTACAAGTTACGCACAGAAGTTCCAGGGCAGAGTCACCATGACCAGGGACACGTCCACGAGCACAGTC |
| | | TACATGGAGCTGAGCAGCCTGAGATCTGAGGACACGGCCGTGTATTACTGTGCGAGAGATGGGTATGGCAGCTTCTCCC |
| | | GGCTGATCCAGCTCTGGGGCCAGGGCACCCTGGTCACCGTCTCCTCA |
| 93 | T0736H VH | QVQLIQSGAKVKKPGASVKVSCKASRYKFTSYYMHWVRQAPGQGLEWMGIINPKSGSTSYAQKFQGRVTMTRDTSTSTV |
| | | YMELSSLRSEDTAVYYCARDGYGSFSRLIQLWGQGTLVTVSS |
| 94 | Nucleic acid | CAGGTTCAGCTGATTCAGTCTGGCGCCGAAGTGAAGAAACCTGGCGCCTCTGTGAAGGTGTCCTGCAAGGCCTCTCGGT |
| | encoding T0736H- | ACAAGTTCACCTCCTACTACATGCACTGGGTCCGACAGGCCCCTGGACAAGGATTGGAGTGGATGGGCATCATCAACCC |
| | IgG4-P E356K | CAAGTCCGGCTCCACCTCTTACGCCCAGAAATTCCAGGGCAGAGTGACCATGACCAGAGACACCTCTACCTCCACCGTG |
| | heavy chain | TACATGGAACTGTCCAGCCTGAGATCCGAGGACACCGCCGTGTACTACTGTGCCAGAGATGGCTACGGCAGCTTCTCCA |
| | | GGCTGATCCAGTTGTGGGGACAGGGCACACTGGTCACCGTGTCCTCTGCTTCTACCAAGGGACCCAGCGTGTTCCCTCT |
| | | GGCTCCTTGCTCCAGATCCACCTCCGAGTCTACAGCTGCTCTGGGCTGCCTGGTCAAGGACTACTTTCCTGAGCCTGTG |
| | | ACCGTGTCTTGGAACTCTGGCGCTCTGACATCTGGCGTGCACACCTTTCCAGCTGTGCTGCAGTCCTCCGGCCTGTACT |
| | | CTCTGTCCTCTGTCGTGACCGTGCCTTCCAGCTCTCTGGGAACCCAGACCTACACCTGTAATGTGGACCACAAGCCTTC |
| | | CAACACCAAGGTGGACAAGCGCGTGGAATCTAAGTACGGCCCTCCTTGTCCTCCATGTCCTGCTCCAGAGTTTCTCGGC |
| | | GGACCCTCCGTGTTTCTGTTCCCTCCAAAGCCTAAGGACACCCTGATGATCTCTCGGACCCCTGAAGTGACCTGCGTGG |
| | | TGGTGGATGTGTCCCAAGAGGATCCCGAGGTGCAGTTCAATTGGTACGTGGACGGCGTGGAAGTGCACAACGCCAAGAC |
| | | CAAGCCTAGAGAGGAACAGTACAACTCCACCTACAGAGTGGTGTCCGTGCTGACCGTGCTGCACCAGGATTGGCTGAAC |
| | | GGCAAAGAGTACAAGTGCAAGGTGTCCAACAAGGGCCTGCCTAGCTCCATCGAAAAGACCATCTCCAAGGCCAAGGGCC |
| | | AGCCTCGAGAACCCCAGGTTTACACCCTGCCTCCAAGCCAGAAAGAGATGACCAAGAACCAGGTGTCCCTGACCTGCCT |
| | | CGTGAAGGGCTTCTACCCTTCCGATATCGCCGTGGAATGGGAGAGCAATGGCCAGCCAGAGAACAACTACAAGACCACA |
| | | CCTCCTGTGCTGGACTCCGATGGCTCATTCTTTCTGTACTCCAAGCTGACAGTGGACAAGTCCCGGTGGCAAGAGGGCA |
| | | ACGTGTTCTCCTGCTCTGTGATGCACGAGGCCCTGCACAACCACTACACCCAGAAGTCCCTGTCTCTGTCCCCT |
| 95 | T0736H-IgG4-P | QVQLIQSGAEVKKPGASVKVSCKASRYKFTSYYMHWVRQAPGQGLEWMGIINPKSGSTSYAQKFQGRVTMTRDTSTSTV |
| | E356K amino acid | YMELSSLRSEDTAVYYCARDGYGSFSRLIQLWGQGTLVTVSSASTKGPSVFPLAPCSRSTSESTAALGCLVKDYFPEPV |
| | sequence | TVSWNSGALTSGVHTFPAVLQSSGLYSLSSVVTVPSSSLGTQTYTCNVDHKPSNTKVDKRVESKYGPPCPPCPAPEFLG |
| | | GPSVFLFPPKPKDTLMISRTPEVTCVVVDVSQEDPEVQFNWYVDGVEVHNAKTKPREEQYNSTYRVVSVLTVLHQDWLN |
| | | GKEYKCKVSNKGLPSSIEKTISKAKGQPREPQVYTLPPSQKEMTKNQVSLTCLVKGFYPSDIAVEWESNGQPENNYKTT |
| | | PPVLDSDGSFFLYSKLTVDKSRWQEGNVFSCSVMHEALHNHYTQKSLSLSP |
| 96 | N0128L VL domain | TCCTATGTGCTGACTCAGCCACCCTCAGTGTCAGTGGCCCCAGGAGAGACGGCCAGGATTACCTGTGGGGGAGACAACA |
| | coding nucleotide | TTGGAAGGAAAAGTGTGTACTGGTACCAGCAGAAGTCAGGCCAGGCCCCTGTGCTGGTCATCTATTATGATAGCGACCG |
| | sequence | GCCCTCAGGGATCCCTGAGCGATTCTCTGGGTCCAACTCTGGGAACACGGCGACCCTGACCATCAGCAGGGTCGAAGCC |
| | | GGGGATGAGGCCGACTATTACTGTCAGGTGTGGGATGGAAGTAGTGATCATTGGGTGTTCGGCGGAGGGACCAAGTTGA |
| | | CCGTCCTAG |
| 97 | N0128L VL domain | SYVLTQPPSVSVAPGETARITCGGDNIGRKSVYWYQQKSGQAPVLVIYYDSDRPSGIPERFSGSNSGNTATLTISRVEA |
| | amino acid | GDEADYYCQVWDGSSDHWVFGGGTKLTVL |
| | sequence | |
| 98 | N0128L-IgL λ | TCCTATGTGC TGACTCAGCC ACCCTCAGTG TCAGTGGCCC CAGGAGAGAC GGCCAGGATT ACCTGTGGGG |
| | light chain | GAGACAACAT TGGAAGGAAA AGTGTGTACT GGTACCAGCA GAAGTCAGGC CAGGCCCCTG TGCTGGTCAT |
| | coding nucleic | CTATTATGAT AGCGACCGGC CCTCAGGGAT CCCTGAGCGA TTCTCTGGGT CCAACTCTGG GAACACGGCG |
| | acid | ACCCTGACCA TCAGCAGGGT CGAAGCCGGG GATGAGGCCG ACTATTACTG TCAGGTGTGG GATGGAAGTA |
| | | GTGATCATTG GGTGTTCGGC GGAGGGACCA AGTTGACCGT CCTAGGTCAG CCCAAGGCTG CCCCCTCGGT |
| | | CACTCTGTTC CCACCCTCCT CTGAGGAGCT TCAAGCCAAC AAGGCCACAC TGGTGTGTCT CATAAGTGAC |
| | | TTCTACCCGG GAGCCGTGAC AGTGGCCTGG AAGGCAGATA GCAGCCCCGT CAAGGCGGGA GTGGAGACCA |
| | | CCACACCCTC CAAACAAAGC AACAACAAGT ACGCGGCCAG CAGCTACCTG AGCCTGACGC CTGAGCAGTG |
| | | GAAGTCCCAC AAAAGCTACA GCTGCCAGGT CACGCATGAA GGGAGCACCG TGGAGAAGAC AGTGGCCCCT |
| | | ACAGAATGTT CA |
| 99 | N0128L λ light | SYVLTQPPSVSVAPGETARITCGGDNIGRKSVYWYQQKSGQAPVLVIYYDSDRPSGIPERFSGSNSGNTATLTISRVEA |
| | chain amino acid | GDEADYYCQVWDGSSDHWVFGGGTKLTVLGQPKAAPSVTLFPPSSEELQANKATLVCLISDFYPGAVTVAWKADSSPVK |
| | sequence (mature) | AGVETTTPSKQSNNKYAASSYLSLTPEQWKSHKSYSCQVTHEGSTVEKTVAPTECS |
| 100 | N0325L VL domain | TACGTGCTGACCCAGCCTCCTTCCGTGTCTGTTGCTCCTGGCGAGACAGCCAGAATCACCTGTGGCGGCGATAACATCG |
| | coding nucleotide | GCCGGAAGTCCGTGTACTGGTATCAGCAGAAGTCCGGCCAGGCTCCTGTGCTGGTCATCTACTACGACTCCGACCGGCC |
| | sequence | TTCTGGCATCCCTGAGAGATTCTCCGGCTCCAACTCCGGCAATACCGCCACACTGACCATCTCCAGAGTGGAAGCTGGC |
| | | GACGAGGCCGACTACTACTGCCAAGTGTGGGACGGCTCCTCTGACCACTGGGTTTTCGGCGGAGGCACCAAGCTGACAG |
| | | TGCTG |
| 101 | N0325L VL domain | YVLTQPPSVSVAPGETARITCGGDNIGRKSVYWYQQKSGQAPVLVIYYDSDRPSGIPERFSGSNSGNTATLTISRVEAG |
| | amino acid | DEADYYCQVWDGSSDHWVFGGGTKLTVL |
| | sequence | |
| 102 | N0325L-IgL λ | TACGTGCTGACCCAGCCTCCTTCCGTGTCTGTTGCTCCTGGCGAGACAGCCAGAATCACCTGTGGCGGCGATAACATCG |
| | light chain coding | GCCGGAAGTCCGTGTACTGGTATCAGCAGAAGTCCGGCCAGGCTCCTGTGCTGGTCATCTACTACGACTCCGACCGGCC |
| | nucleic acid; | TTCTGGCATCCCTGAGAGATTCTCCGGCTCCAACTCCGGCAATACCGCCACACTGACCATCTCCAGAGTGGAAGCTGGC |
| | N0325L coding | GACGAGGCCGACTACTACTGCCAAGTGTGGGACGGCTCCTCTGACCACTGGGTTTTCGGCGGAGGCACCAAGCTGACAG |
| | sequence | TGCTGGGACAACCTAAGGCCGCTCCTTCTGTGACCCTGTTTCCTCCATCCTCCGAGGAACTGCAGGCCAACAAGGCTAC |
| | underlined | CCTCGTGTGCCTGATCTCCGACTTTTACCCTGGCGCTGTGACCGTGGCCTGGAAGGCTGATAGTTCTCCTGTGAAGGCC |
| | | GGCGTGGAAACCACCACACCTTCCAAGCAGTCCAACAACAAATACGCCGCCTCCTCCTACCTGTCTCTGACCCCTGAAC |
| | | AGTGGAAGTCCCACAAGTCCTACTCTTGCCAAGTGACCCACGAGGGCTCCACCGTGGAAAAGACAGTGGCTCCTACCGA |
| | | GTGCTCC |
| 103 | N0325L λ light | YVLTQPPSVSVAPGETARITCGGDNIGRKSVYWYQQKSGQAPVLVIYYDSDRPSGIPERFSGSNSGNTATLTISRVEAG |
| | chain amino acid | DEADYYCQVWDGSSDHWVFGGGTKLTVLGQPKAAPSVTLFPPSSEELQANKATLVCLISDFYPGAVTVAWKADSSPVKA |
| | sequence (mature) | GVETTTPSKQSNNKYAASSYLSLTPEQWKSHKSYSCQVTHEGSTVEKTVAPTECS |
| 104 | IgG4 PE human | ASTKGPSVFPLAPCSRSTSESTAALGCLVKDYFPEPVTVSWNSGALTSGVHTFPAVLQSSGLYSLSSVVTVPSSSLGTK |
| | heavy chain | TYTCNVDHKPSNTKVDKRVESKYGPPCPPCPAPEFEGGPSVFLFPPKPKDTLMISRTPEVTCVVVDVSQEDPEVQFNWY |
| | constant region | VDGVEVHNAKTKPREEQFNSTYRVVSVLTVLHQDWLNGKEYKCKVSNKGLPSSIEKTISKAKGQPREPQVYTLPPSQEE |
| | | MTKNQVSLTCLVKGFYPSDIAVEWESNGQPENNYKTTPPVLDSDGSFFLYSRLTVDKSRWQEGNVFSCSVMHEALHNHY |
| | | TQKSLSLSLGK |
| 105 | IgG4 human heavy | ASTKGPSVFPLAPCSRSTSESTAALGCLVKDYFPEPVTVSWNSGALTSGVHTFPAVLQSSGLYSLSSVVTVPSSSLGTK |
| | chain constant | TYTCNVDHKPSNTKVDKRVESKYGPPCPPCPAPEFEGGPSVFLFPPKPKDTLMISRTPEVTCVVVDVSQEDPEVQFNWY |
| | region with knobs- | VDGVEVHNAKTKPREEQFNSTYRVVSVLTVLHQDWLNGKEYKCKVSNKGLPSSIEKTISKAKGQPREPQVCTLPPSQEE |
| | into-holes | MTKNQVSLWCLVKGFYPSDIAVEWESNGQPENNYKTTPPVLDSDGSFFLYSRLTVDKSRWQEGNVFSCSVMHEALHNHY |
| | mutations and | TQKSLSLSLGK |
| | hinge mutation. | |
| | Type a (IgG4ra) | |
| 106 | IgG4 human heavy | ASTKGPSVFPLAPCSRSTSESTAALGCLVKDYFPEPVTVSWNSGALTSGVHTFPAVLQSSGLYSLSSVVTVPSSSLGTK |
| | chain constant | TYTCNVDHKPSNTKVDKRVESKYGPPCPPCPAPEFEGGPSVFLFPPKPKDTLMISRTPEVTCVVVDVSQEDPEVQFNWY |
| | region with knobs- | VDGVEVHNAKTKPREEQFNSTYRVVSVLTVLHQDWLNGKEYKCKVSNKGLPSSIEKTISKAKGQPREPQVYTLPPSQCE |
| | into-holes | MTKNQVSLSCAVKGFYPSDIAVEWESNGQPENNYKTTPPVLDSDGSFFLVSRLTVDKSRWQEGNVFSCSVMHEALHNHY |
| | mutations and | TQKSLSLSLGK |
| | hinge mutation. | |
| | Type b (IgG4yb) | |
| 107 | IgG4 human | ASTKGPSVFPLAPCSRSTSESTAALGCLVKDYFPEPVTVSWNSGALTSGVHTFPAVLQSSGLYSLSSVVTVPSSSLGTQ |
| | heavy chain | TYTCNVDHKPSNTKVDKRVESKYGPPCPPCPAPEFLGGPSVFLFPPKPKDTLMISRTPEVTCVVVDVSQEDPEVQFNWY |
| | constant region | VDGVEVHNAKTKPREEQYNSTYRVVSVLTVLHQDWLNGKEYKCKVSNKGLPSSIEKTISKAKGQPREPQVYTLPPSQEE |
| | with P (hinge) | MTKNQVSLTCLVKGFYPSDIAVEWESNGQPENNYKTTPPVLDSDGSFFLYSKLTVDKSRWQEGNVFSCSVMHEALHNHY |
| | mutation and K439E | TQESLSLSP |
| 108 | IgG4 human | ASTKGPSVFPLAPCSRSTSESTAALGCLVKDYFPEPVTVSWNSGALTSGVHTFPAVLQSSGLYSLSSVVTVPSSSLGTQ |
| | heavy chain | TYTCNVDHKPSNTKVDKRVESKYGPPCPPCPAPEFLGGPSVFLFPPKPKDTLMISRTPEVTCVVVDVSQEDPEVQFNWY |
| | constant region | VDGVEVHNAKTKPREEQYNSTYRVVSVLTVLHQDWLNGKEYKCKVSNKGLPSSIEKTISKAKGQPREPQVYTLPPSQKE |
| | with P (hinge) | MTKNQVSLTCLVKGFYPSDIAVEWESNGQPENNYKTTPPVLDSDGSFFLYSKLTVDKSRWQEGNVFSCSVMHEALHNHY |
| | mutation and E356K | TQKSLSLSP |
| 109 | IgG4-P K439E | GCTTCCACCAAGGGACCCAGCGTTTTCCCTCTGGCTCCTTGCTCCAGATCCACCTCCGAGTCTACAGCTGCTCTGGGCT |
| | encoding nucleic | GCCTGGTCAAGGACTACTTTCCTGAGCCTGTGACCGTGTCCTGGAACTCTGGCGCTCTGACATCTGGCGTGCACACCTT |
| | acid | TCCAGCTGTGCTGCAGTCCTCCGGCCTGTACTCTCTGTCCTCTGTCGTGACCGTGCCTTCCAGCTCTCTGGGAACCCAG |
| | | ACCTACACCTGTAATGTGGACCACAAGCCTTCCAACACCAAGGTGGACAAGCGCGTGGAATCTAAGTACGGCCCTCCTT |
| | | GTCCTCCATGTCCTGCTCCAGAGTTTCTCGGCGGACCCTCCGTGTTTCTGTTCCCTCCAAAGCCTAAGGACACCCTGAT |
| | | GATCTCTCGGACCCCTGAAGTGACCTGCGTGGTGGTGGATGTGTCCCAAGAGGATCCCGAGGTGCAGTTCAATTGGTAC |
| | | GTGGACGGCGTGGAAGTGCACAATGCCAAGACCAAGCCTAGAGAGGAACAGTACAACTCCACCTACAGAGTGGTGTCCG |
| | | TGCTGACCGTGCTGCACCAGGATTGGCTGAACGGCAAAGAGTACAAGTGCAAGGTGTCCAACAAGGGCCTGCCTAGCTC |
| | | CATCGAAAAGACCATCTCCAAGGCCAAGGGCCAGCCTCGAGAACCCCAGGTTTACACCCTGCCTCCAAGCCAAGAGGAA |
| | | ATGACCAAGAACCAGGTGTCCCTGACCTGTCTCGTGAAGGGCTTCTACCCCTCCGATATCGCCGTGGAATGGGAGTCTA |
| | | ATGGCCAGCCAGAGAACAACTACAAGACCACACCTCCAGTGCTGGACTCCGACGGCTCATTCTTTCTGTACTCCAAGCT |
| | | GACAGTGGACAAGTCCCGGTGGCAAGAGGGCAACGTGTTCTCCTGCTCTGTGATGCACGAGGCCCTGCACAACCACTAC |
| | | ACCCAAGAGTCCCTGTCTCTGTCCCCT |
| 110 | IgG4-P E356K | GCTTCCACCAAGGGACCCAGCGTGTTCCCTCTGGCTCCTTGCTCCAGATCCACCTCCGAGTCTACAGCTGCTCTGGGCT |
| | encoding nucleic | GCCTGGTCAAGGACTACTTTCCTGAGCCTGTGACCGTGTCTTGGAACTCTGGCGCTCTGACATCTGGCGTGCACACCTT |
| | acid | TCCAGCTGTGCTGCAGTCCTCCGGCCTGTACTCTCTGTCCTCTGTCGTGACCGTGCCTTCCAGCTCTCTGGGAACCCAG |
| | | ACCTACACCTGTAATGTGGACCACAAGCCTTCCAACACCAAGGTGGACAAGCGCGTGGAATCTAAGTACGGCCCTCCTT |
| | | GTCCTCCATGTCCTGCTCCAGAGTTTCTCGGCGGACCCTCCGTGTTTCTGTTCCCTCCAAAGCCTAAGGACACCCTGAT |
| | | GATCTCTCGGACCCCTGAAGTGACCTGCGTGGTGGTGGATGTGTCCCAAGAGGATCCCGAGGTGCAGTTCAATTGGTAC |
| | | GTGGACGGCGTGGAAGTGCACAACGCCAAGACCAAGCCTAGAGAGGAACAGTACAACTCCACCTACAGAGTGGTGTCCG |
| | | TGCTGACCGTGCTGCACCAGGATTGGCTGAACGGCAAAGAGTACAAGTGCAAGGTGTCCAACAAGGGCCTGCCTAGCTC |
| | | CATCGAAAAGACCATCTCCAAGGCCAAGGGCCAGCCTCGAGAACCCCAGGTTTACACCCTGCCTCCAAGCCAGAAAGAG |
| | | ATGACCAAGAACCAGGTGTCCCTGACCTGCCTCGTGAAGGGCTTCTACCCTTCCGATATCGCCGTGGAATGGGAGAGCA |
| | | ATGGCCAGCCAGAGAACAACTACAAGACCACACCTCCTGTGCTGGACTCCGATGGCTCATTCTTTCTGTACTCCAAGCT |
| | | GACAGTGGACAAGTCCCGGTGGCAAGAGGGCAACGTGTTCTCCTGCTCTGTGATGCACGAGGCCCTGCACAACCACTAC |
| | | ACCCAGAAGTCCCTGTCTCTGTCCCCT |
| 111 | Nucleic acid | GGACAACCTAAGGCCGCTCCTTCTGTGACCCTGTTTCCTCCATCCTCCGAGGAACTGCAGGCCAACAAGGCTACCCTCG |
| | encoding IgL | TGTGCCTGATCTCCGACTTTTACCCTGGCGCTGTGACCGTGGCCTGGAAGGCTGATAGTTCTCCTGTGAAGGCCGGCGT |
| | human lambda | GGAAACCACCACACCTTCCAAGCAGTCCAACAACAAATACGCCGCCTCCTCCTACCTGTCTCTGACCCCTGAACAGTGG |
| | light chain | AAGTCCCACAAGTCCTACTCTTGCCAAGTGACCCACGAGGGCTCCACCGTGGAAAAGACAGTGGCTCCTACCGAGTGCT |
| | constant region | CC |
| 112 | Human lambda | GQPKAAPSVTLFPPSSEELQANKATLVCLISDFYPGAVTVAWKADSSPVKAGVETTTPSKQSNNKYAASSYLSLTPEQW |
| | light chain | KSHKSYSCQVTHEGSTVEKTVAPTECS |
| | constant region | |
| 113 | Human kappa | KRTVAAPSVFIFPPSDEQLKSGTASVVCLLNNFYPREAKVQWKVDNALQSGNSQESVTEQDSKDSTYSLSSTLTLSKAD |
| | light chain | YEKHKVYACEVTHQGLSSPVTKSFNRGEC |
| | constant region | |
| 116 | LARI VL domain | QAGLTQPPSVSKGLRQTATLTCTGNSNNVGNLGAAWLQQHQGHPPKLLSYRNNNRPSGISERFFASRSGNTASLTITGL |
| | amino acid | QPEDEADYYCSAWDSSLSAWVFGGGTKLTVL |
| | sequence | |
| 117 | Truncated LARI | GLTQPPSVSKGLRQTATLTCTGNSNNVGNLGAAWLQQHQGHPPKLLSYRNNNRPSGISERFFASRSGNTASLTITGLQP |
| | VL domain amino | EDEADYYCSAWDSSLSAWVFGGGTKLTVL |
| | acid sequence | |
| 122 | LARI VL domain | CAGGCAGGGCTGACTCAGCCACCCTCGGTGTCCAAGGGCTTGAGACAGACCGCCACACTCACCTGCACTGGGAACAGCA |
| | coding nucleotide | ACAATGTTGGCAACCTAGGAGCAGCTTGGCTGCAGCAGCACCAGGGCCACCCTCCCAAACTCCTATCCTACAGGAATAA |
| | sequence | TAACCGGCCCTCAGGGATCTCAGAGAGATTCTTTGCATCCAGGTCAGGAAACACAGCCTCCCTGACCATTACTGGACTC |
| | | CAGCCTGAGGACGAGGCTGACTATTACTGCTCAGCATGGGACAGCAGCCTCAGTGCTTGGGTGTTCGGCGGAGGGACCA |
| | | AGCTGACCGTCCTA |

TABLE U
Nucleotide sequences of known human vλ10-54 alleles. IMGT
identifies vλ10-54*03 as a pseudogene.

| IGLV10-54 | |
|---|---|
| allele | Nucleotide sequence |
| Vλ10-54*01 | CAGGCAGGGCTGACTCAGCCACCCTCGGTGTCCAAGGGCTTGAGACAGACCGCCACACTC |
| SEQ ID NO: 118 | ACCTGCACTGGGAACAGCAACAATGTTGGCAACCAAGGAGCAGCTTGGCTGCAGCAGCAC |
| | CAGGGCCACCCTCCCAAACTCCTATCCTACAGGAATAACAACCGGCCCTCAGGGATCTCA |
| | GAGAGATTATCTGCATCCAGGTCAGGAAACACAGCCTCCCTGACCATTACTGGACTCCAG |
| | CCTGAGGACGAGGCTGACTATTACTGCTCAGCATGGGACAGCAGCCTCAGTGCTCA |
| Vλ10-54*02 | CAGGCAGGGCTGACTCAGCCACCCTCGGTGTCCAAGGGCTTGAGACAGACCGCCACACTC |
| D86996 | ACCTGCACTGGGAACAGCAACATTGTTGGCAACCAAGGAGCAGCTTGGCTGCAGCAGCAC |
| SEQ ID NO: 119 | CAGGGCCACCCTCCCAAACTCCTATCCTACAGGAATAACAACCGGCCCTCAGGGATCTCA |
| | GAGAGATTCTCTGCATCCAGGTCAGGAAACACAGCCTCCCTGACCATTACTGGACTCCAG |
| | CCTGAGGACGAGGCTGACTATTACTGCTCAGCATTGGACAGCAGCCTCAGTGCTCA |
| Vλ10-54*03 | TCCTATGTGCTGACTCAGCCACCCTCGGTGTCAGTGGCCCCAGGACAGACGGCCAGGATT |
| S70116 | ACCTGTGGGGGAAACAACATTGGAAGTAAAAGTGTGCACTGGTACCAGCAGAAGCCAGGC |
| SEQ ID NO: 120 | CAGGCCCCTGTGCTGGTCGTCTATGATGATAGCGACCGGCCCTCAGGGATCCCTGAGCGA |
| | TTCTCTGGCTCCAACTCTGGGAACACGGCCACCCTGACCATCAGCAGGGTCGAAGCCGGG |
| | GATGAGGCCGACTATTACTGTCAGGTGTGGGATAGTAGTAGTGATCATCC |

What is claimed is:

1. A polypeptide comprising an antibody light chain variable (VL) domain with an N-terminal signal peptide, wherein
the VL domain comprises a sequence derived from a v λ gene segment and a sequence derived from a j gene segment, wherein
the N-terminal signal peptide differs from the native N-terminal signal peptide for said λ v gene segment, and wherein
the VL domain comprises an N-terminal truncation relative to the amino acid sequence encoded by the v λ gene segment starting at IMGT position 1.

2. A polypeptide according to claim 1, wherein the residue corresponding to IMGT position 1 of the VL domain is absent and the N-terminal residue of the VL domain corresponds to IMGT position 2.

3. A polypeptide according to claim 1 or claim 2, wherein the v λ gene segment is a human v λ gene segment.

4. A polypeptide according to any preceding claim, wherein the j gene segment is a human j λ gene segment.

5. A polypeptide according to any of claims 1 to 4, wherein the first encoded residue (IMGT position 1) of the v λ gene segment is Ser, optionally wherein the v λ gene segment is a vertebrate IGLV3 family gene segment.

6. A polypeptide according to claim 5, wherein the v λ gene segment is IGLV3-21, IGLV3-1, IGLV3-9, IGLV3-10, IGLV3-12, IGLV3-13, IGLV3-16, IGLV3-19, IGLV3-22, IGLV3-25 or IGLV3-27.

7. A polypeptide according to claim 5 or claim 6, wherein the first two encoded residues (IMGT positions 1 to 2) of the v λ gene segment are Ser Tyr.

8. A polypeptide according to claim 7, wherein the first three encoded residues (IMGT positions 1 to 3) of the v λ gene segment are Ser Tyr Val.

9. A polypeptide according to claim 6 or claim 7, wherein the v λ gene segment is human IGLV3-21.

10. A polypeptide according to any preceding claim, wherein the signal peptide has a length of 20 amino acids.

11. A polypeptide according to any preceding claim, wherein the signal peptide is not a human λ v gene segment signal peptide.

12. A polypeptide according to claim 11, wherein the signal peptide is not a λ v gene segment signal peptide.

13. A polypeptide according to any preceding claim, wherein the signal peptide comprises a C-terminal Cys residue.

14. A polypeptide according to claim 13, wherein the signal peptide comprises a C-terminal sequence Ala Arg Cys.

15. A polypeptide according to claim 14, wherein the signal peptide comprises a C-terminal sequence Thr Asp Ala Arg Cys.

16. A polypeptide according to any preceding claim, wherein the signal peptide is a κ v gene segment signal peptide.

17. A polypeptide according to claim 16, wherein the signal peptide is a mouse κ v gene segment signal peptide.

18. A polypeptide according to claim 17, wherein the signal peptide is SEQ ID NO: 62.

19. A polypeptide according to any of claims 1 to 4, wherein the signal peptide is SEQ ID NO: 74.

20. A polypeptide according to any preceding claim, wherein the polypeptide is an antibody light chain comprising the VL domain and a light chain constant (CL) domain, optionally wherein the CL domain is a λCL domain.

21. A polypeptide according to any preceding claim, wherein the VL domain is the N0325L VL domain SEQ ID NO: 101.

22. A polypeptide according to claim 21, consisting of amino acid sequence SEQ ID NO: 66 or comprising said sequence as an N-terminal domain.

23. A polypeptide according to claim 22, which is an antibody λ light chain consisting of or comprising amino acid sequence SEQ ID NO: 67.

24. A polypeptide according to any of claims 1 to 20, wherein the VL domain is the LARI VL domain SEQ ID NO: 117.

25. Nucleic acid encoding a polypeptide according to any preceding claim.

26. Nucleic acid according to claim 25, wherein the nucleic acid is cDNA.

27. Nucleic acid according to claim 25, wherein the nucleic acid is genomic DNA comprising introns.

28. Nucleic acid according to any of claims 25 to 27, comprising nucleotide sequence SEQ ID NO: 64 encoding the signal peptide.

29. Nucleic acid according to any of claims 25 to 28, comprising nucleotide sequence SEQ ID NO: 100 encoding the VL domain.

30. Nucleic acid according to claim 29, comprising nucleotide sequence SEQ ID NO: 65.

31. A host cell in vitro comprising nucleic acid according to any of claims 25 to 30.

32. A cell according to claim 31 which is a mammalian cell.

33. A cell according to claim 32 which is a CHO cell.

34. A cell according to claim 31 which is a human cell, optionally a HEK cell.

35. A cell according to any of claims 31 to 34, wherein said nucleic acid is integrated into chromosomal DNA of the cell.

36. An in vitro population of cells according to any of claims 31 to 35.

37. A population of cells according to claim 36, wherein
the cells express the encoded polypeptide, and wherein
on expression in the cells, the signal peptide is cleaved from the polypeptide to provide a mature VL domain comprising an N-terminal residue corresponding to the N-terminal residue of a VL domain encoded by the same v λ gene segment when expressed with and cleaved from its native signal peptide, optionally wherein the N-terminal residue is IMGT position 2 or IMGT position 3.

38. A method of expressing a polypeptide comprising a VL domain, the method comprising
culturing a population of cells according to claim 36 or claim 37 under conditions for expression of the polypeptide, wherein
the N-terminal signal peptide is cleaved off the VL domain to provide a polypeptide comprising a mature VL domain, wherein
the N-terminal residue of the polypeptide comprising the mature VL domain is corresponds to the N-terminal residue of a VL domain encoded by the same v λ gene segment when expressed with and cleaved from its native signal peptide, optionally wherein the N-terminal residue is IMGT position 2 or IMGT position 3 of the VL domain.

39. A method of expressing an antibody comprising a VH domain and a VL domain, wherein the VL domain comprises a sequence derived from a v λ gene segment and a sequence derived from a j gene segment, the method comprising
providing a population of cells according to claim 36 or claim 37, wherein the cells further comprise nucleic acid encoding the VH domain,
culturing said population of cells under conditions for expression of the polypeptide comprising the VL domain and for expression of the VH domain, wherein
the N-terminal signal peptide is cleaved off the VL domain to provide a polypeptide comprising a mature VL domain, wherein
the N-terminal residue of the polypeptide comprising the mature VL domain corresponds to the N-terminal residue of a VL domain encoded by the same v λ gene segment when expressed with and cleaved from its native signal peptide, optionally wherein the N-terminal residue is IMGT position 2 or IMGT position 3 of the VL domain, and wherein
the VL domain assembles with the VH domain to provide said antibody.

40. A method according to claim 38 or claim 39, wherein the polypeptide or antibody is secreted from the cells.

41. A method according to any of claims 38 to 40, comprising isolating the polypeptide or antibody from the population of cells.

42. A method according to claim 41, comprising purifying the polypeptide or antibody by one or more steps of protein chromatography.

43. A method according to any of claims 38 to 42, wherein the antibody is an IgG.

44. A method according to any of claims 38 to 43, wherein the antibody is a multispecific antibody.

45. A method of expressing a bispecific antibody comprising two heavy chains each comprising a different VH domain, and a common light chain comprising a VL domain, wherein the VL domain comprises a sequence derived from a v λ gene segment and a sequence derived from a j gene segment, the method comprising
providing a population of cells according to claim 36 or claim 37, wherein the cells further comprise nucleic acid encoding the two heavy chains,
culturing said population of cells under conditions for expression of the antibody heavy and light chains, wherein
the N-terminal signal peptide is cleaved off the VL domain to provide an antibody light chain comprising a mature VL domain, wherein
the N-terminal residue of the polypeptide comprising the mature VL domain corresponds to the N-terminal residue of a VL domain encoded by the same v λ gene segment when expressed with and cleaved from its native signal peptide, optionally wherein the N-terminal residue is IMGT position 2 or IMGT position 3 of the VL domain, and wherein
the light chain domain assembles with each of the two heavy chains to provide said bispecific antibody.

46. A method according to claim 45, wherein the N-terminal residue of the polypeptide comprising the mature VL domain is IMGT position 2 of the VL domain.

47. A method according to claim 46, wherein the two heavy chains comprise VH domains N1441H SEQ ID NO: 83 and T0999H SEQ ID NO: 87 respectively and wherein the light chain comprises VL domain 0325L SEQ ID NO: 101.

48. A method according to claim 47, wherein the antibody is an IgG4 comprising the N1441H heavy chain SEQ ID NO: 85, the T0999H heavy chain SEQ ID NO: 89 and the 0325L light chain SEQ ID NO: 103.

49. A method according to any of claims 45 to 48, wherein the signal peptide is SEQ ID NO: 62.

50. An antibody VL domain obtained by cleavage of the N-terminal signal peptide from the polypeptide of any of claims 1 to 24.

51. An isolated antibody comprising a VL domain according to claim 50.

52. An isolated antibody obtained by the method of any of claims 39 to 49.

53. A method of improving stability and/or antigen-binding, or of reducing immunogenicity, of an antibody comprising a VH domain and a VL domain, wherein the VL domain comprises a sequence derived from a v λ gene segment and a sequence derived from a j gene segment, the method comprising
providing nucleic acid encoding said antibody, comprising a nucleotide sequence encoding said VL domain with an N-terminal signal peptide, wherein the codon or codons for the residue or residues corresponding to IMGT position 1 and optionally IMGT position 2 of said VL domain is or are deleted, and wherein the N-terminal signal peptide is not the native N-terminal signal peptide for said λ v gene segment, and
expressing said nucleic acid to provide an antibody comprising the VH domain and VL domain, wherein the N-terminal residue of the VL domain is the residue corresponding to IMGT position 2 or IMGT position 3, and optionally isolating and purifying said antibody.

54. A method comprising
(i) providing a nucleotide sequence encoding an antibody light chain variable (VL) domain,
(ii) providing a first DNA molecule comprising the said nucleotide sequence preceded by a leader sequence encoding the native signal peptide,
(iii) providing a second DNA molecule comprising the said nucleotide sequence preceded by a leader sequence encoding a non-native signal peptide,
(iv) expressing the first and second DNA in host cells to produce first and second polypeptides respectively comprising the VL domain,
(v) comparing VL domain amino acid sequences of the first and second polypeptides expressed from the host cells,
(vi) identifying an N-terminal truncation in the sequence of the first polypeptide compared with the second polypeptide,
(vii) providing an engineered nucleotide sequence encoding the sequence of the first polypeptide, including the N-terminal truncation,
(viii) providing a third DNA molecule comprising the said engineered nucleotide sequence preceded by a leader sequence encoding a non-native signal peptide, and
(ix) expressing the third DNA molecule in a host cell to produce a third polypeptide comprising the encoded VL domain, wherein the amino acid sequence of the third polypeptide is identical to that of the first polypeptide.

55. A method according to claim 54, comprising
(x) isolating and purifying the third polypeptide from the host cell.

56. A method according to claim 54 or claim 55, comprising expressing an antibody comprising a VH domain and the said VL domain.

57. A method according to claim 56, wherein an IgG antibody is produced by co-expressing an antibody heavy chain comprising the VH domain and an antibody light chain comprising the VL domain in the host cells.
